SEQUENCE LISTING (24 sequences)

SEQ ID NO: 1 - protein, 618 aa, Bacillus subtilis
  misc_feature: Full length Bacillus subtilis AmyE amino acid sequence
LTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLDRALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTATKLPDGRYDNKAGAGSFQVNDGKLTGTINARSVAVLYPDDIAKAPHVFLENYKTGVTHSFNDQLTITLRADANTTKAVYQINNGPETAFKDGDQFTIGKGDPFGKTYTIMLKGTNSDGVTRTEKYSFVKRDPASAKTIGYQNPNHWSQVNAYIYKHDGSRVIELTGSWPGKPMTKNADGIYTLTLPADTDTTNAKVIFNNGSAQVPGQNQPGFDYVLNGLYNDSGLSGSLPH

SEQ ID NO: 2 - DNA, 1854 bp, Bacillus subtilis
  misc_feature: Nucleotide sequence encoding the AmyE of SEQ ID NO 1
cttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatcagcagagttccaatacggagaaatccttcaggatagtgcctccagagatgctgcatatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccat

SEQ ID NO: 3 - protein, 425 aa, Bacillus subtilis
  misc_feature: Truncated Bacillus subtilis AmyE (AmyE-tr) amino acid sequence. The native signal sequence is not shown.
LTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLDRALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTATKLPDGRYDNKAGAGSFQVNDGKLTGTINARSVAVLYPD

SEQ ID NO: 4 - DNA, 1275 bp, Bacillus subtilis
  misc_feature: Nucleotide sequence encoding AmyE-tr of SEQ ID NO 2
cttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatcagcagagttccaatacggagaaatccttcaggatagtgcctccagagatgctgcatatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgat

SEQ ID NO: 5 - protein, 659 aa, Bacillus subtilis
  misc_feature: Bacillus subtilis alpha-amylase variant Amy31A amino acid sequence (UniProtKB/TrEMBL Accession No. O82953)
MFEKRFKTSLLPLFAGFLLLFHLVLSGPAAANAETANKSNKVTASSVKNGTILHAWNWSFNTLTQNMKDIRDAGYAAIQTSPINQVKEGNQGDKSMSNWYWLYQPTSYQIGNRYLGTEQEFKDMCAAAEKYGVKVIVDAVVNHTTSDYGAISDEIKRIPNWTHGNTQIKNWSDRWDITQNALLGLYDWNTQNTEVQAYLKGFLERALNDGADGFRYDAAKHIELPDDGNYGSQFWPNITNTSAEFQYGEILQDSASRDTAYANYMNVTASNYGHSIRSALKNRILSVSNISHYASDVSADKLVTWVESHDTYANDDEESTWMSDDDIRLGWAVIGSRSGSTPLFFSRPEGGGNGVRFPGKSQIGDRGSALFKDQAITAVNQFHNEMAGQPEELSNPNGNNQIFMNQRGSKGVVLANAGSSSVTINTSTKLPDGRYDNRAGAGSFQVANGKLTGTINARSAAVLYPDDIGNAPHVFLENYQTEAVHSFNDQLTVTLRANAKTTKAVYQINNGQETAFKDGDRLTIGKEDPIGTTYNVKLTGTNGEGASRTQEYTFVKKDPSQTNIIGYQNPDHWGNVNAYIYKHDGGGAIELTGSWPGKAMTKNADGIYTLTLPANADTADAKVIFNNGSAQVPGQNHPGFDYVQNGLYNNSGLNGYLPH

SEQ ID NO: 6 - DNA, 1842 bp, Bacillus subtilis
  misc_feature: Nucleotide sequence encoding B. subtilis Amy31A of SEQ ID NO 5
tctgttaaaaacggcactattctgcatgcatggaactggagctttaacacgctgacccagaacatgaaagatattcgtgacgcgggctatgctgcgatccaaaccagccctatcaaccaggtcaaagaaggcaaccaaggcgacaaatccatgtccaactggtactggctgtatcaaccgacgtcctatcagattggcaaccgttatctgggcacggagcaagagttcaaagacatgtgtgctgcggctgagaaatatggtgtgaaagttatcgtggacgctgtggtaaaccacacgacctctgattatggtgctattagcgacgagattaaacgtattccaaattggacccatggtaatacccagatcaaaaattggagcgaccgctgggacattacccagaatgcgctgctgggtctgtatgactggaacacgcaaaacaccgaagtacaggcatatctgaagggcttcctggaacgcgctctgaacgatggtgctgatggttttcgctacgacgccgcaaagcatattgagctgccggatgacggcaactacggttcccaattctggccgaacatcaccaacacctctgccgaattccagtacggcgagatcctgcaagactccgcgagccgtgacaccgcttatgccaactatatgaacgtaactgcctctaactatggccattccattcgttctgcgctgaaaaatcgtatcctgtccgtgtccaatatctcccactatgcatccgacgtttctgctgacaaactggtaacttgggtcgagtctcacgacacctatgcaaatgatgacgaggagagcacctggatgagcgatgatgatattcgtctgggttgggcggttattggttctcgctctggttctactccgctgttctttagccgtccggaaggtggcggcaatggcgttcgtttcccgggtaaatctcaaattggtgatcgtggctctgcactgtttaaagatcaagctattacggcggtgaatcagttccataatgagatggcaggtcaacctgaagaactgtccaatccaaacggtaacaaccaaatcttcatgaaccagcgtggcagcaaaggcgtcgtcctggcgaacgccggtagctcttctgttaccatcaacacgtctaccaaactgccagacggccgctatgataaccgtgcgggtgctggttcctttcaggtagccaacggcaagctgacgggcaccatcaacgctcgttctgctgctgttctgtacccggacgacattggcaacgctccgcacgtgttcctggagaattaccagaccgaagcggtacatagctttaatgaccagctgaccgtcactctgcgtgccaacgcaaaaaccacgaaagcagtctatcagatcaataatggtcaagaaactgctttcaaggatggcgaccgtctgactattggtaaggaggacccgattggcaccacttataacgttaaactgactggcaccaatggcgagggcgctagccgcactcaagagtatacgttcgtaaagaaagacccgtctcaaaccaacatcatcggttaccagaatcctgaccactggggtaatgtgaacgcttacatctataaacatgatggtggcggtgctatcgaactgaccggctcttggccaggtaaagccatgacgaaaaacgcggatggcatctataccctgaccctgccggccaatgcggataccgcagatgcgaaggttatcttcaataacggctccgcgcaggttccgggccaaaaccatccgggctttgactacgtacaaaatggtctgtataacaactctggcctgaacggttacctgccgcac

SEQ ID NO: 7 - protein, 549 aa, Geobacillus stearothermophilus
  misc_feature: Full length Geobacillus stearothermophilus alpha-amylase (AmyS; P06279) protein sequence
MLTFHRIIRKGWMFLLAFLLTALLFCPTGQPAKAAAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 8 - protein, 512 aa, Bacillus licheniformis
  misc_feature: Full length Bacillus licheniformis alpha- amylase (AmyL; P06278) protein sequence
MKQQKRLYARLLTLLFALIFLLPHSAAAAANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHRIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNSTVVSKHPLKAVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 9 - protein, 660 aa, Bacillus subtilis
  misc_feature: Full length Bacillus subtilis alpha-amylase (AmyE; NP_388186)
MFAKRFKTSLLPLFAGFLLLFHLVLAGPAAASAETANKSNELTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLDRALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTATKLPDGRYDNKAGAGSFQVNDGKLTGTINARSVAVLYPDDIAKAPHVFLENYKTGVTHSFNDQLTITLRADANTTKAVYQINNGPDDRRLRMEINSQSEKEIQFGKTYTIMLKGTNSDGVTRTEKYSFVKRDPASAKTIGYQNPNHWSQVNAYIYKHDGSRVIELTGSWPGKPMTKNADGIYTLTLPADTDTTNAKVIFNNGSAQVPGQNQPGFDYVLNGLYNDSGLSGSLPH

SEQ ID NO: 10 - protein, 659 aa, Bacillus subtilis
  misc_feature: Full length Bacillus subtilis alpha-amylase (AmyE; NCBI Accession No. ABW75769)
MFAKRFKTSLLPLFAGFLLLFHLVLAGPAAASAETANKSNELTAPSIKSGTILHAWNWSFNTLKHNMKDIHDAGYTAIQTSPINQVKEGNQGNKSMSNWYWLYQPTSYQIGNRYLGTEQEFKEMCAAAEEYGIKVIVDAVINHTTSDYAAISNEIKSIPNWTHGNTQIKNWSDRWDVTQNSLLGLYDWNTQNTQVQSYLKRFLERALNDGADGFRFDAAKHIELPDDGSYGSQFWPNITNTSAEFQYGEILQDSASRDAAYANYMNVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTPTKLPDGRYDNKAGAGSFQVNDGKLTGTINARSVAVLYPDDIAKAPHVFLENYKTGVTHSFNDQLTITLRADANTTKAVYQINNGPETAFKDGDQFTIGKGDPFGKTYTIMLKGTNSNGVTKAEEYSFVKRDPASAKTIGYQNPNHWSQVNAYIYKHDGSRAIELTGSWPGKPMTKNADGIYTLTLPADTDTTNAKVIFNNGSAQVPGQNQPGFDYVQNGLYNDSGLSGSLPH

SEQ ID NO: 11 - protein, 659 aa, Bacillus subtilis
  misc_feature: Full length Bacillus subtilis alpha-amylase (AmyE; NCBI Accession No. ABK54355)
MFAKRFKTSLLPLFAGFLLLFHLVLAGPAAASAETANKSNELTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEIKSIPNWTHGNTQIKNWSDRWDVTQNSLLGLYDWNTQNTQVQSYLKRFLERALNDGADGFRFDAAKHIELPDDGSYGSQFWPTITNTSAEFQYGEILQDSASRDAAYANYMDVTASNYGHSIRSALKNRNLGVSNLSHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTATKLPDGRYDNKAGAGSFQVNDGKLTGTINARSVAVLYPDDIAKAPHVFLENYKTGVTHSFNDQLTITLRADANTTKAVYQINNGPETAFKDGDQFTIGKGDPFGKTYTIMLKGTNSDGVTRAEEYSFVKRDPASAKTIGYQNPNHWSQVNAYIYKHDGGRAIELTGSWPGKPMTKNADGIYTLTLPADTDTTNAKVIFNNGSAQVPGQNQPGFDYVQNGLYNDSGLSGSLPH

SEQ ID NO: 12 - protein, 659 aa, Bacillus subtilis; identical to SEQ ID NO: 11
  misc_feature: Full length Bacillus subtilis alpha-amylase (AmyE; NCBI Accession No. AAF14358)

SEQ ID NO: 13 - protein, 659 aa, Bacillus subtilis
  misc_feature: Full length Bacillus subtilis alpha-amylase (AmyE; NCBI Accession No. AAT01440)
MFAKRFKTSLLPLFAGFLLLFHLVLAGPAAASAETANKSNELTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLERALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTPTKLPDGRYDNKAGAGSFQVNDGKLTGTINARSVAVLYPDDIAQAPHVFLENYKTGVTHSFNDQLTITLRADANTTKAVYQINNGPETAFKDGDQFTIGKGDPFGKTYTIMLKGTNSDGVTRTEEYSFIKRDPASAKTIGYQNPNHWSQVNAYIYKHDGGQAIELTGSWPGKPMTKNADGIYTLTLPADTDTTNAKVIFNNGSAQVPGQNQPGFDYVQNGLYNDSGLSGSLPY

SEQ ID NO: 14 - protein, 659 aa, Bacillus subtilis
  misc_feature: Full length Bacillus subtilis alpha-amylase (AmyE; NCBI Accession No. AAZ30064)
MFAKRFKTSLLPLFAGFLLLFHLVLAGPNAANAETANKSNELTAPSIKSGTILHAWNWSFNTLKHNMKDIHDAGYTAIQTSPINQVKEGNQGNKSMLNWYWLYQPTSYQIGNRYLGTEQEFKEMCAAAEEYGIKVIVDAVINHTTSDYAAISNEIKSIPNWTHGNTQIKNWSDRWDVTQNSLLGLYDWNTQNTQVQSYLKRFLERALNDGADGFRFDAAKHIELPDDGSYGSQFWPNITNTSAEFQYGEILQDSASRDASYANYMNVTASNYGHSIRSALKNRNLGVSNISHYASDVPADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTPTKLPDGRYDNKAGAGSFQVNDGKLTGTINARSVAVLYPDDIAKAPHVFLENYKTGVTHSFNDQLTITMRADAKTTKAVYQINNGPETAFKDGDQFTIGKGDPFGKTYTIMLKGTNSDGVTRTEEYSFIKRDPASAKTIGYQNPNHWSQVNAYIYKHDGGQAIELTGSWPGKPMTKNADGIYTLTLPADTDTTNAKVIFNNGSAQVPGQNQPGFDYVQNGLYNDSGLSGSLPH

SEQ ID NO: 15 - protein, 649 aa, Bacillus subtilis
  misc_feature: Full length Bacillus subtilis alpha-amylase (AmyE NCBI Accession No. AAQ83841)
MFAKRFKTSLLPLFAGFLLLFYLVLAGPAAASAETANKSIELTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLDRALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRISHGVVLANAGSSSVSINTATKLPDGRYDNKAGAGSFQVNDGKLTGTINARSVAVLYPDDIAKAPHVFLENYKTGVTHSFNDQLTITLRADANTFIKSIMDQINXRXRRLRMEINSQSEKEIQFGKTYTIMLKGTNSDGVTRXEKYSLPKRDPASAKTIGYQNPNHWSQVNAYIYKHDGSREIELTGSWPGKPMTKNADGIYTLTLPADTDTTNAKVIFNNGYAQVPGQNQPGFDYVLNGLY

SEQ ID NO: 16 - protein, 659 aa, Bacillus subtilis; identical to SEQ ID NO: 5
  misc_feature: Full length Bacillus subtilis alpha-amylase (AmyE; NCBI Accession No. BAA31528)

SEQ ID NO: 17 - protein, 41 aa, Bacillus subtilis
  misc_feature: Native signal sequence of the AmyE of SEQ ID NO 1
MFAKRFKTSLLPLFAGFLLLFHLVLAGPAAASAETANKSNE

SEQ ID NO: 18 - DNA, 60 bp, Artificial Sequence
  Synthetic Primer PSTAMYE-F
cttcttgctgcctcattctgcagcttcagcacttacagcaccgtcgatcaaaagcggaac

SEQ ID NO: 19 - DNA, 57 bp, Artificial Sequence
  Synthetic Primer AMYENOPST-R
ctggaggcactatcctgaaggatttctccgtattggaactctgctgatgtatttgtg

SEQ ID NO: 20 - DNA, 57 bp, Artificial Sequence
  Synthetic Primer AMYENOPST-F
cacaaatacatcagcagagttccaatacggagaaatccttcaggatagtgcctccag

SEQ ID NO: 21 - DNA, 56 bp, Artificial Sequence
  Synthetic
caggaaatccgtcctctgttaactcaatggggaagagaaccgcttaagcccgagtc

SEQ ID NO: 22 - DNA, 55 bp, Artificial Sequence
  Synthetic Primer HPAIAMYE466-R
caggaaatccgtcctctgttaactcaatcaggataaagcacagctacagacctgg

SEQ ID NO: 23 - DNA, 22 bp, Artificial Sequence
  Synthetic Primer AMYE SEQ-F1
tacacaagtacagtcctatctg

SEQ ID NO: 24 - DNA, 22 bp, Artificial Sequence
  Synthetic Primer AMYE SEQ-F2
catcctctgtctctatcaatac

(19) United States
(12) Patent Application Publication
(10) Pub. No.: US 2015/0079660 A1
(43) Pub. Date: Mar. 19, 2015

(54) METHODS FOR REDUCING BLUE SACCHARIDE

(71) Applicant: DANISCO US INC., Palo Alto, CA (US)

(72) Inventors: Vivek Sharma, North Liberty, IA (US); Jayarama Shetty, Pleasanton, CA (US); Bruce Strohm, Beloit, WI (US)

(21) Appl. No.: 14/494,436

(22) Filed: Sep. 23, 2014

Related U.S. Application Data
(63) Continuation of application No. 13/501,236, filed on Apr. 10, 2012, now abandoned.
(60) Provisional application No. 61/254,626, filed on Oct. 23, 2009.

(51) Int. Cl. C07H 1/08; C12N 9/28

(52) U.S. Cl. CPC C07H 1/08; C12N 9/2417
USPC 435/202; 435/276

(57) ABSTRACT

The present disclosure relates to a Bacillus subtilis alpha-amylase (AmyE) or its variant thereof. AmyE or its variants thereof may be used to eliminate or reduce the iodine-positive starch presented in saccharide liquor. Also disclosed are a composition comprising an AmyE or variant thereof and a method utilizing an AmyE or variant thereof to eliminate or reduce the iodine-positive starch

### CROSS-REFERENCE TO RELATED APPLICATIONS

This application is a Continuation of U.S. patent application Ser. No. 13/501,236, filed Apr. 10, 2012, which is a 371 National Phase of PCT/US2010/053196, filed on Oct. 19, 2010, which claims the benefit of U.S. Provisional Patent Application Ser. No. 61/254,626 filed Oct. 23, 2009, the contents of which are incorporated herein by reference in their entirety.

### SEQUENCE LISTING

The sequence listing submitted via EFS, in compliance with 37 C.F.R. §1.52(e), is incorporated herein by reference. The sequence listing text file submitted via EFS contains the file “2010-12-16 31419WO_ST25.txt”, created on Nov. 25, 2014, which is 81,920 bytes in size.

### FIELD OF THE INVENTION

A composition comprising a Bacillus subtilis alpha-amylase (AmyE) or variant thereof is useful in eliminating iodine-positive starch (IPS) in a saccharide liquor, for example. Methods of using an AmyE or variant thereof to eliminate IPS, for example, are also provided.

### BACKGROUND

Vegetable starches, e.g., cornstarch, are widely used in the industrial manufacture of products such as syrups and biofuels. For example, high fructose corn syrup (HFCS) is a processed form of corn syrup having high fructose content and a sweetness comparable to sugar, making HFCS useful as a sugar substitute in soft drinks and other processed foods. HFCS production currently represents a billion-dollar industry. Similarly, the production of ethanol from vegetable starches is a rapidly expanding industry. Ethanol has widespread applications as an industrial chemical, a gasoline additive, or a liquid fuel by itself. The use of ethanol as a fuel or fuel additive statistically significantly reduces air emissions while maintaining or even improving engine performance. On the other hand, ethanol is a renewable fuel, so that its use may reduce dependence on finite fossil fuel sources. Furthermore, use of ethanol may decrease the net accumulation of carbon dioxide in the atmosphere.

Syrups and biofuels can be produced from starch by an enzymatic process that catalyzes the breakdown of starch into glucose. This enzymatic process typically involves a sequence of enzyme-catalyzed reactions:

(1) Liquefaction: Alpha-amylases (EC 3.2.1.1) first catalyze the degradation of a starch suspension, which may contain 30-40% w/w dry solids (ds), to maltodextrans. Alpha-amylases are endohydrolases that catalyze the random cleavage of internal α-1,4-D-glucosidic bonds. Because liquefaction typically is conducted at high temperatures, e.g., 90-100° C., thermostable alpha-amylases, such as an alpha-amylase from Bacillus sp., are preferred for this step. Alpha-amylases currently used for this step, e.g., alpha-amylases from B. licheniformis (AmyL), B. amyloliquefaciens, and Geobacillus stearothermophilus (AmyS), do not produce significant amounts of glucose. Instead, the resulting liquefact has a low dextrose equivalent (DE), and contains maltose and sugars with high degrees of polymerization (DPn).

(2) Saccharification: Glucoamylases and/or maltogenic alpha-amylases catalyze the hydrolysis of non-reducing ends of the maltodextrans formed after liquefaction, releasing D-glucose, maltose and isomaltose. Saccharification produces saccharide liquor, which is either glucose-rich or high-maltose. In the former case, glucoamylases typically catalyze saccharification under acidic conditions at elevated temperatures, e.g., 60° C., pH 4.3. Glucoamylases used in this process typically are obtained from fungi, e.g., Aspergillus niger glucoamylase used in Optidex® L400 (Danisco US Inc., Genencor Division) or Humicola grisea glucoamylase. De-branching enzymes, such as pullulanases, can aid saccharification.

Maltogenic alpha-amylases alternatively may catalyze saccharification to form high-maltose syrups. Maltogenic alpha-amylases typically have a higher optimal pH and a lower optimal temperature than glucoamylase; and maltogenic amylases typically require Ca²⁺. Maltogenic alpha-amylases currently used for this application include B. subtilis alpha-amylases, plant amylases, and alpha-amylase from Aspergillus oryzae, the active ingredient of Clarase® L (Danisco US Inc., Genencor Division). Exemplary saccharification reactions used to produce various products are depicted below:

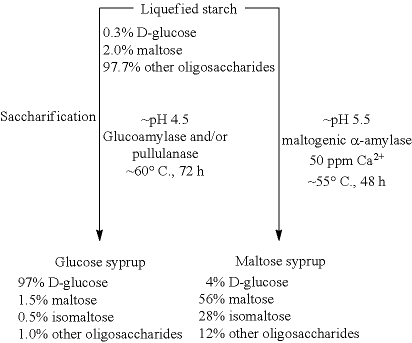

(3) Further processing: A branch point in the process occurs after the production of a glucose-rich syrup, shown on the left side of the reaction pathways above. If the final desired product is a biofuel, yeast can ferment the glucose-rich syrup to ethanol. On the other hand, if the final desired product is a fructose-rich syrup, glucose isomerase can catalyze the conversion of the glucose-rich syrup to fructose.

Industrial starch-processing facilities occasionally encounter process excursions, e.g., temperature, pH, slurry flow rate, or enzyme dose, any of which may result in the presence of a significant amount of iodine-positive starch (IPS) in saccharide liquor. IPS, which comes from amylose that escapes hydrolysis and/or retrograded starch polymer, is able to react with iodine to produce a characteristic blue/purple color. IPS-containing saccharide liquor is thus called a blue saccharide. The presence of IPS in saccharide liquor negatively affects final product quality and represents a major issue with downstream processing. Typically, the presence of IPS can be remedied by isolating the saccharification tank and blending the contents back at a level that is undetectable. Although such a remedy is able to reduce the IPS level below the detection by customers, the offending material is still there and will accumulate in carbon columns and filter systems, among other things. Particularly, there is no post-saccharification method to effectively eliminate or minimize IPS by a selective degradation. Accordingly, it is desirable to develop an enzymatic solution that enables effective elimination or reduction of IPS upon the detection of IPS post-saccharification.

### SUMMARY

The alpha-amylase from Bacillus subtilis, AmyE, exhibits properties different from the Termamyl-like alpha-amylases, such as the alpha-amylases from Bacillus licheniformis and Bacillus stearothermophilus. AmyE has a previously unrecognized transglucosidase activity and is able to synthesize maltotriose from maltose. Additionally, AmyE has been found to produce significant amounts of glucose from various carbohydrate substrates. Adding AmyE, or a variant thereof, and a glucoamylase to saccharification results in, among other things, a higher level of fermentable sugars, and a reduced level of higher sugars. Furthermore, use of AmyE or variant thereof in saccharification, for example, statistically significantly improves the quality of the resulting saccharide liquor, which is suitable for production of high fructose corn syrup (HFCS) or ethanol from starch.

The embodiment contemplated herein provides a composition for eliminating or reducing iodine-positive starch. The composition comprises an alpha-amylase that is effective eliminating or reducing iodine-positive starch present in the saccharide liquor. Also provided is a method of eliminating or reducing iodine-positive starch. The method comprises contacting a saccharide liquor containing iodine-positive starch with the alpha-amylase or the contemplated composition comprising of the alpha-amylase. Optionally, the method may further comprise contacting a phytase with the saccharide liquor. The iodine-positive starch may result from process excursions of temperature, pH, enzyme dose, or any combination thereof.

The alpha-amylase as contemplated herein may be a Bacillus subtilis alpha-amylase (AmyE) having an amino acid sequence of SEQ ID NO: 1 or an alpha-amylase having at least about 80%, about 85%, about 90%, about 95%, or about 99% sequence identity to SEQ ID NO: 1. The alpha-amylase may comprises SEQ ID NO: 1 or consists of SEQ ID NO:1. Optionally, the alpha-amylase may comprises an amino acid sequence set forth in SEQ ID NO: 3, SEQ ID NO: 5, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. The alpha-amylase may also be an AmyE variant, which has one or more altered properties compared to the AmyE having an amino acid sequence of SEQ ID NO: 1. The one or more altered properties of the alpha-amylase may include: substrate specificity, substrate binding, substrate cleavage pattern, thermal stability, pH/activity profile, pH/stability profile, stability towards oxidation, stability at lower level of calcium ion (Ca²⁺), specific activity, or any combination thereof.

In one aspect, the alpha-amylase may be used at an amount of about 0.1 to about 0.4 mg per gram of starch (mg/g starch) in the method contemplated herein to eliminate or reduce iodine-positive starch. In another aspect, the contemplated method may be performed at a pH about 5.0 to about 5.5. In a further aspect, the contemplated method may be performed at a temperature about 58° C. to about 62° C. In yet another aspect, the contemplated method may be performed for about 4 to about 24 hours.

### BRIEF DESCRIPTION OF THE DRAWINGS

The accompanying drawings are incorporated into the specification and provide non-limiting illustrations of various embodiments. In the drawings:

FIG. 1 depicts amino acid sequence alignment of full-length alpha-amylases (with signal sequences) from Geobacillus stearothermophilus (SEQ ID NO: 7; AmyS; “B. stear”), Bacillus licheniformis (SEQ ID NO: 8; AmyL; “B. lich”), and Bacillus subtilis (SEQ ID NO: 9; AmyE; “B. sub”).

FIG. 2 depicts a three-dimensional structure comparison between B. subtilis alpha-amylase (AmyE; Protein Data Bank Accession No. 1UA7) and G. stearothermophilus alpha-amylase (AmyS; Protein Data Bank Accession No. 1HVX).

FIG. 3A depicts the superposed structures of G. stearothermophilus alpha-amylase (AmyS; Protein Data Bank Accession No. 1HVX) (gray shaded) and B. licheniformis (AmyL; Protein Data Bank Accession No. 1BLI) (dark shaded). The left panel shows an overall comparison, while the right panel shows a magnified view of selected amino acid side chains. FIG. 3B depicts a stereographic view of the superposed structures of G. stearothermophilus alpha-amylase (AmyS; Protein Data Bank Accession No. 1HVX) (gray shaded) and B. subtilis alpha-amylase (AmyE; Protein Data Bank Accession No. 1UA7) (dark shaded).

FIG. 4 depicts plasmid pME630-7, which comprises a polynucleotide (labeled “SAMY 425aa”) that encodes AmyE-tr (SEQ ID NO: 3). The plasmid comprises a polynucleotide in-frame with the SAMY gene that encodes a signal sequence from B. licheniformis alpha-amylase (labeled “pre LAT”).

FIGS. 5A and 5B depict the HPLC analysis of reaction products catalyzed by AmyE during incubation with maltose. FIG. 5A shows the result without AmyE addition. The AmyE-mediated maltotriose synthesis result, shown in FIG. 5B, is catalyzed by the transglucosidase activity.

FIG. 6 depicts the DE development of liquefaction reactions catalyzed by (1) Fuelzyme®-LF at pH 4.6, (2) GC 358 at pH 5.8 at 108.5° C., and (3) GC 358 at pH 5.25 at 106.7° C. The first and third liquefaction reactions are known to produce poor liquefact, which results in iodine-positive starch after normal saccharification.

FIG. 7 depicts iodine test results for saccharide liquors from (1) Fuelzyme®-LP liquefact, (2) GC 358 (good cook) liquefact, and (3) GC 358 (bad cook) at 112-hour time point. The saccharification condition and AmyE treatment of the various liquefacts are described in Example 3 and Tables 2-3.

FIG. 8 depicts iodine test results for saccharide liquors from (1) Fuelzyme®-LF liquefact, (2) GC 358 (good cook) liquefact, and (3) GC 358 (bad cook) at 136-hour time point. The saccharification condition and AmyE treatment of the various liquefacts are described in Example 3 and Tables 2-3.

FIG. 9 depicts sediment test results for saccharide liquors from (1) Fuelzyme®-LF liquefact, (2) GC 358 (good cook) liquefact, and (3) GC 358 (bad cook) at 136-hour time point. The saccharification condition and AmyE treatment of the various liquefacts are described in Example 3 and Tables 2-3. The sediment test is described in Example 2.

FIG. 10 depicts iodine test results for IPS-containing saccharide liquor treated with (1) AmyE, (2) Clarase® L, and (3) G-ZYME® G 998 for 4, 8.5, and 24 hours. The absorbance at 520 nm, indicating the relative amount of IPS, is plotted against the time of treatment. Exemplary liquefaction conditions, saccharification conditions, and treatments by various alpha-amylases are described in Example 4 and Table 5.

FIG. 11 depicts iodine test results for IPS-containing saccharide liquor treated with (1) AmyE, (2) Clarase® L, and (3) G-ZYME® G 998 for 4 hours. Exemplary liquefaction conditions, saccharification conditions, and treatments by various alpha-amylases are provided in Example 4 and Table 5.

FIG. 12 depicts iodine test results for IPS-containing saccharide liquor treated with AmyE for 8.5 and 24 hours. Exemplary liquefaction conditions, saccharification conditions, and treatments by various alpha-amylases are described in Example 4 and Table 5.

FIG. 13 depicts the DP1 level in the IPS-containing saccharide liquor treated with (1) AmyE, (2) Clarase® L, and (3) G-ZYME® G 998. Exemplary liquefaction conditions, saccharification conditions, and treatments by various alpha-amylases are described in Example 4 and Table 5.

FIG. 14 depicts the DP2 level in the IPS-containing saccharide liquor treated with (1) AmyE, (2) Clarase® L, and (3) G-ZYME® G 998. Exemplary liquefaction conditions, saccharification conditions, and treatments by various alpha-amylases are described in Example 4 and Table 5.

### DETAILED DESCRIPTION

The present disclosure relates to a Bacillus subtilis alpha-amylase (AmyE). The AmyE or its variant thereof may be used to eliminate or reduce iodine-positive starch (IPS) present in saccharide liquor. Also disclosed include a composition comprising the AmyE or its variant thereof and a method of eliminating or reducing iodine-positive starch utilizing the AmyE or its variant.

### 1. DEFINITIONS AND ABBREVIATIONS

In accordance with this detailed description, the following abbreviations and definitions apply. It should be noted that as used herein, the singular forms “a,” “an,” and “the” include plural referents unless the context clearly dictates otherwise. Thus, for example, reference to “an enzyme” includes a plurality of such enzymes and reference to “the formulation” includes reference to one or more formulations and equivalents thereof known to those skilled in the art, and so forth.

Unless defined otherwise, all technical and scientific terms used herein have the same meaning as commonly understood by one of ordinary skill in the art. The following terms are provided below.

### 1.1. Definitions

As used herein, “amino acid sequence” is synonymous with the term “polypeptide” and/or the term “protein.” In some instances, the term “amino acid sequence” is synonymous with the term “peptide”; in some instances, the term “amino acid sequence” is synonymous with the term “enzyme.”

As used herein, “hybridization” includes the process by which a strand of nucleic acid joins with a complementary strand through base pairing, as well as the process of amplification as carried out in polymerase chain reaction (PCR) technologies. Hybridized nucleic acid may exist as single- or double-stranded DNA or RNA, an RNA/DNA heteroduplex, or an RNA/DNA copolymer. As used herein, “copolymer” refers to a single nucleic acid strand that comprises both ribonucleotides and deoxyribonucleotides. Nucleic acids include those that hybridize under “highly stringent conditions” to a nucleic acid disclosed herein. Highly stringent conditions are defined as hybridization at 50° C. in 0.2×SSC or at 65° C. in 0.1×SSC (1×SSC=0.15 M NaCl, 0.015 M sodium citrate, pH 7.0).

As used herein, “nucleotide sequence” or “nucleic acid sequence” refer to a sequence of genomic, synthetic, or recombinant origin and may be double-stranded or single-stranded, whether representing the sense or anti-sense strand. As used herein, the term “nucleic acid” may refer to genomic DNA, cDNA, synthetic DNA, or RNA. The residues of a nucleic acid may contain any of the chemically modifications commonly known and used in the art.

“Isolated” means that the material is at least substantially free from at least one other component that the material is naturally associated and found in nature.

“Purified” means that the material is in a relatively pure state, e.g., at least about 90% pure, at least about 95% pure, or at least about 98% pure.

“Thermostable” means the enzyme retains activity after exposure to elevated temperatures. The thermostability of an alpha-amylase is measured by its half-life (t_{1/2}), where half of the enzyme activity is lost by the half-life. The half-life is measured by determining the specific alpha-amylase activity of the enzyme remaining over time at a given temperature, particularly at a temperature used for a specific application.

As used herein, “food” includes both prepared food, as well as an ingredient for a food, such as flour, that is capable of providing any beneficial effect to the consumer. “Food ingredient” includes a formulation that is or can be added to a food or foodstuff and includes formulations used at low levels in a wide variety of products that require, for example, acidifying or emulsifying. The food ingredient may be in the form of a solution or as a solid, depending on the use and/or the mode of application and/or the mode of administration.

“Oligosaccharide” means a carbohydrate molecule composed of 3-20 monosaccharides.

“Homologue” means an entity having a certain degree of identity or “homology” with the subject amino acid sequences or the subject nucleotide sequences. A “homologous sequence” is used in the manner of “percent identity.” It is meant to include an amino acid sequence having at least 85% sequence identity to the subject sequence, e.g., at least 85%, 90%, 95%, 96%, 97%, 98% or 99% identity to the subject sequence. Typically, homologues will comprise the same active site residues as the subject amino acid sequence.

As used herein, “transformed cell” includes cells that have been transformed by use of recombinant DNA techniques. Transformation typically occurs by insertion of one or more nucleotide sequences into a cell. The inserted nucleotide sequence may be a heterologous nucleotide sequence, i.e., is a sequence that is not natural to the cell that is to be transformed, such as a fusion protein.

As used herein, “operably linked” means that the described components are in a relationship permitting them to function in their intended manner. For example, a regulatory sequence operably linked to a coding sequence is ligated in such a way that expression of the coding sequence is achieved under condition compatible with the control sequences.

As used herein, “biologically active” refers to a sequence having a similar structural, regulatory, or biochemical function as the naturally occurring sequence, although not necessarily to the same degree.

As used herein, “starch” refers to any material comprised of the complex polysaccharide carbohydrates of plants, comprised of amylose and amylopectin with the formula (C₆H₁₀O₅)ₓ, wherein “X” can be any number. In particular, the term refers to any plant-based material including but not limited to grains, grasses, tubers and roots and more specifically wheat, barley, corn, rye, rice, sorghum, brans, cassava, millet, potato, sweet potato, and tapioca.

As used herein, “granular starch” refers to uncooked (raw) starch, which has not been subject to gelatinization.

As used herein, “starch gelatinization” means solubilization of a starch molecule to form a viscous suspension.

As used herein, “gelatinization temperature” refers to the lowest temperature at which gelatinization of a starch substrate occurs. The exact temperature depends upon the specific starch substrate and further may depend on the particular variety and the growth conditions of plant species from which the starch is obtained.

“DE” or “dextrose equivalent” is an industry standard for measuring the concentration of total reducing sugars, calculated as the percentage of the total solids that have been converted to reducing sugars. The granular starch that has not been hydrolyzed has a DE that is about zero (0), and D-glucose has a DE of about 100.

As used herein, “starch substrate” refers to granular starch or liquefied starch using refined starch, whole ground grains, or fractionated grains.

As used herein, “liquefied starch” refers to starch that has gone through solubilization process, for example, the conventional starch liquefaction process.

As used herein, “glucose syrup” refers to an aqueous composition containing glucose solids. Glucose syrup will have a DE of at least about 20. In some embodiments, glucose syrup may contain no more than about 21% water while at least about 25% reducing sugar calculated as dextrose. In one embodiment, glucose syrup may include at least about 90% D-glucose, and in another embodiment, glucose syrup may include at least about 95% D-glucose. In some embodiments, the terms glucose and glucose syrup are used interchangeably.

As used herein, “fermentable sugars” refer to saccharides that are capable of being metabolized under yeast fermentation conditions. These sugars mainly refer to glucose, maltose, and maltotriose (DP1, DP2 and DP3).

As used herein, “total sugar content” refers to the total sugar content present in a starch composition.

As used herein, “ds” refers to dissolved solids in a solution.

As used herein, “starch-liquefying enzyme” refers to an enzyme that catalyzes the hydrolysis or breakdown of granular starch. Exemplary starch liquefying enzymes include alpha-amylases (EC 3.2.1.1).

“Amylase” means an enzyme that is, among other things, capable of catalyzing the degradation of starch.

“Alpha-amylases (EC 3.2.1.1)” refer to endo-acting enzymes that cleave α-D-(1→4) β-glycosidic linkages within the starch molecule in a random fashion. In contrast, the exo-acting amylolytic enzymes, such as beta-amylases (EC 3.2.1.2; α-D-(1→4)-glucan maltohydrolase) and some product-specific amylases like maltogenic alpha-amylase (EC 3.2.1.133) cleave the starch molecule from the non-reducing end of the substrate. These enzymes have also been described as those effecting the exo- or endohydrolysis of 1,4-α-D-glucosidic linkages in polysaccharides containing 1,4-α-linked D-glucose units. Another term used to describe these enzymes is glycogenase. Exemplary enzymes include alpha-1,4-glucan 4-glucanohydrolase.

As used herein, “glucoamylases” refer to the amyloglucosidase class of enzymes (EC 3.2.1.3, glucoamylase, α-1,4-D-glucan glucohydrolase). These are exo-acting enzymes that release glucosyl residues from the non-reducing ends of amylose and/or amylopectin molecules. The enzymes are also capably of hydrolyzing α-1, 6 and α-1,3 linkages, however, at much slower rates than the hydrolysis of α-1,4 linkages.

As used herein, the “transglucosidase activity” of AmyE or its variants thereof is characterized by the formation of maltotriose upon incubation with maltose. Specifically, the transglucosidase activity refers to the alpha-1,4-glucosyl transferase activity.

One “Modified Wohlgemuth unit” (MWU) refers to the amount of enzyme, e.g., Fuelzyme®-LF, which is able to hydrolyze 1 mg of soluble starch to specific dextrins under standard reaction conditions in 30 minutes. See also Diversa Corp., URL at www.diversa.com/pdf/Fuelzyme-LF_Brochure.pdf.

One “FTU” (phytase unit) refers to the amount of phytase that is able to liberate 1 μmol of inorganic phosphate in one minute at 37.2° C. and pH 5.5.

As used herein, “iodine-positive starch” or “IPS” refers to (1) amylose that is not hydrolyzed after liquefaction and saccharification, or (2) a retrograded starch polymer. When saccharified starch or saccharide liquor is tested with iodine, the high DPn amylose or the retrograded starch polymer binds iodine and produces a characteristic blue color. The saccharide liquor is thus termed “iodine-positive saccharide,” “blue saccharide,” or “blue sac.” IPS is highly undesirable in starch processing applications, because its presence may reflect incomplete starch hydrolysis and/or interfere with various downstream applications, for example, sweetener production. Specifically, IPS plugs or slows filtration system, and fouls the carbon columns used for purification. When IPS reaches sufficiently high levels, it may leak through the carbon columns and decrease production efficiency. Additionally, it may results in hazy final product upon storage, which is unacceptable for final product quality.

As used herein, formation of “retrograded starch” or “starch retrogradation” refers to the changes that occur spontaneously in a starch paste, or gel on ageing. It arises from the inherent tendency of starch molecules to bind to one another followed by an increase in crystallinity. Solutions of low concentration become increasingly cloudy due to the progressive association of starch molecules into larger articles. Spontaneous precipitation takes place and the precipitated starch appears to be reverting to its original condition of cold-water insolubility. Pastes of higher concentration on cooling set to a gel, which on ageing becomes steadily firmer due to the increasing association of the starch molecules. This arises because of the strong tendency for hydrogen bond formation between hydroxy groups on adjacent starch molecules. See J. A. Radley, ed., STARCH AND ITS DERIVATIVES 194-201 (Chapman and Hall, London (1968)).

As used herein, “hydrolysis of starch” refers to the cleavage of glucosidic bonds with the addition of water molecules.

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP4+ (>DP4) denotes polymers with a degree of polymerization of greater than four.

As used herein, “contacting” or “admixing” refers to the placing of the respective enzyme(s) in sufficiently close proximity to the respective substrate to enable the enzyme(s) to convert the substrate to the end-product. Those skilled in the art will recognize that mixing solutions of the enzyme with the respective substrates can affect contacting or admixing.

### 1.2. Abbreviations

The following abbreviations apply unless indicated otherwise:
AE alcohol ethoxylate
AEO alcohol ethoxylate
AEOS alcohol ethoxysulfate
AES alcohol ethoxysulfate
GAU glucoamylase activity unit
AkAA Aspergillus kawachii alpha-amylase
AmyE Bacillus subtilis alpha-amylase
AmyE-tr AmyE truncated
AmyE FL full length AmyE
AmyL Bacillus licheniformis alpha-amylase
AmyR SPEZYME® XTRA amylase
AmyS Geobacillus stearothermophilus alpha-amylase
AS alcohol sulfate
BAA bacterial alpha-amylase
cDNA complementary DNA
CMC carboxymethylcellulose
DE Dextrose Equivalent
DI distilled, deionized
DNA deoxyribonucleic acid
DP3 degree of polymerization with three subunits
DPn degree of polymerization with n subunits
DS or ds dry solid
dss dry solid starch
DTMPA diethyltriaminepentaacetic acid
EC enzyme commission for enzyme classification
EDTA ethylenediaminetetraacetic acid
EDTMPA ethylenediaminetetramethylene phosphonic acid
EO ethylene oxide
F&HC fabric and household care
FTU phytase unit
g gram
gpm gallon per minute
GAU glucoamylase units
HFCS high fructose corn syrup
HFSS high fructose starch based syrup
HGA Humicola grisea glucoamylase
HPLC high pressure liquid chromatography
IPS iodine-positive starch
IPTG isopropyl β-D-thiogalactoside
IRS insoluble residual starch
kg kilogram
LA Lauria agar
LB Lauria broth
LIT leucine (L) residue at position 1 is replaced with a threonine (T) residue, where amino acids are designated by single letter abbreviations commonly known in the art
MOPS 3-(N-morpholino)propanesulfonic acid
MT metric ton
MW molecular weight
MWU modified Wohlgemuth units
NCBI National Center for Biotechnology Information
nm nanometer
NOBS nonanoyloxybenzenesulfonate
NTA nitrilotriacetic acid
OD optical density
PCR polymerase chain reaction
PEG polyethylene glycol
PI isoelectric point
ppm parts per million
psi pound per square inch
PVA poly(vinyl alcohol)
PVP poly(vinylpyrrolidone)
RAU Reference Amylase Units
RMSD root mean square deviation
RNA ribonucleic acid
RO reverse osmosis
rpm revolutions per minute
SAS secondary alkane sulfonates
1×SSC 0.15 M NaCl, 0.015 M sodium citrate, pH 7.0
SSF simultaneous saccharification and fermentation
SSU soluble starch unit, equivalent to the reducing power of 1 mg of glucose released per minute
TAED tetraacetylethylenediamine
TNBS trinitrobenzenesulfonic acid
TrGA Trichoderma reesei glucoamylase
w/v weight/volume
w/w weight/weight
wt wild-type
μL microliter
Nm microNewton×meter

### 2. AmyE AND OTHER ALPHA-AMYLASES

### 2.1. Structure and Function

Alpha-amylases constitute a group of enzymes present in microorganisms and tissues from animals and plants. They are capable of hydrolyzing alpha-1,4-glucosidic bonds of glycogen, starch, related polysaccharides, and some oligosaccharides. Although all alpha-amylases possess the same catalytic function, their amino acid sequences vary greatly. The sequence identity between different amylases can be virtually non-existent, e.g., falling below about 25%. Despite considerable amino acid sequence variation, alpha-amylases share a common overall topological scheme, which has been identified after the three-dimensional structures of alpha-amylases from different species have been determined. The common three-dimensional structure reveals three domains: (1) a “TIM” barrel known as domain A, (2) a long loop region known as domain B that is inserted within domain A, and (3) a region close to the C-terminus known as domain C that contains a characteristic beta-structure with a Greek-key motif.

The TIM barrel of domain A consists of eight alpha-helices and eight parallel beta-strands, i.e., (β/+)₈, that alternate along the peptide backbone. This structure, named after a conserved glycolytic enzyme triosephosphate isomerase, has been known to be common among conserved protein folds. Domain B is a loop region inserted between β_{A3} and α_{A3} (the third β-strand and α-helix in domain A). Both domain A and domain B are directly involved in the catalytic function of an alpha-amylase, because the three-dimensional structure indicates that domain A flanks the active site and domain overlays the active site from on side. Furthermore, domain A is considered the catalytic domain, as amino acid residues of the active site are located in loops that link beta-strands to the adjacent alpha-helices. Domain B is believed to determine the specificity of the enzyme by affecting substrate binding. MacGregor et al., Biochim. Biophys. Acta. 1546: 1-20 (2001).

“Termamyl-like” alpha-amylases refer to a group of alpha-amylases widely used in the starch-processing industry. The B. licheniformis alpha-amylase having an amino acid sequence of SEQ ID NO: 2 of U.S. Pat. No. 6,440,716 is commercially available as Termamyl®. Termamyl-like alpha-amylases commonly refer to a group of highly homologous alpha-amylases produced by Bacillus spp. Other exemplary members of the group include the alpha-amylases from Geobacillus stearothermophilus (previously known as Bacillus stearothermophilus; both names are used interchangeably in the present disclosure) and B. amyloliquefaciens, and those derived from Bacillus sp. NCIB 12289, NCIB 12512, NCIB 12513, and DSM 9375, all of which are described in detail in U.S. Pat. No. 6,440,716 and WO 95/26397, and incorporated herein by reference.

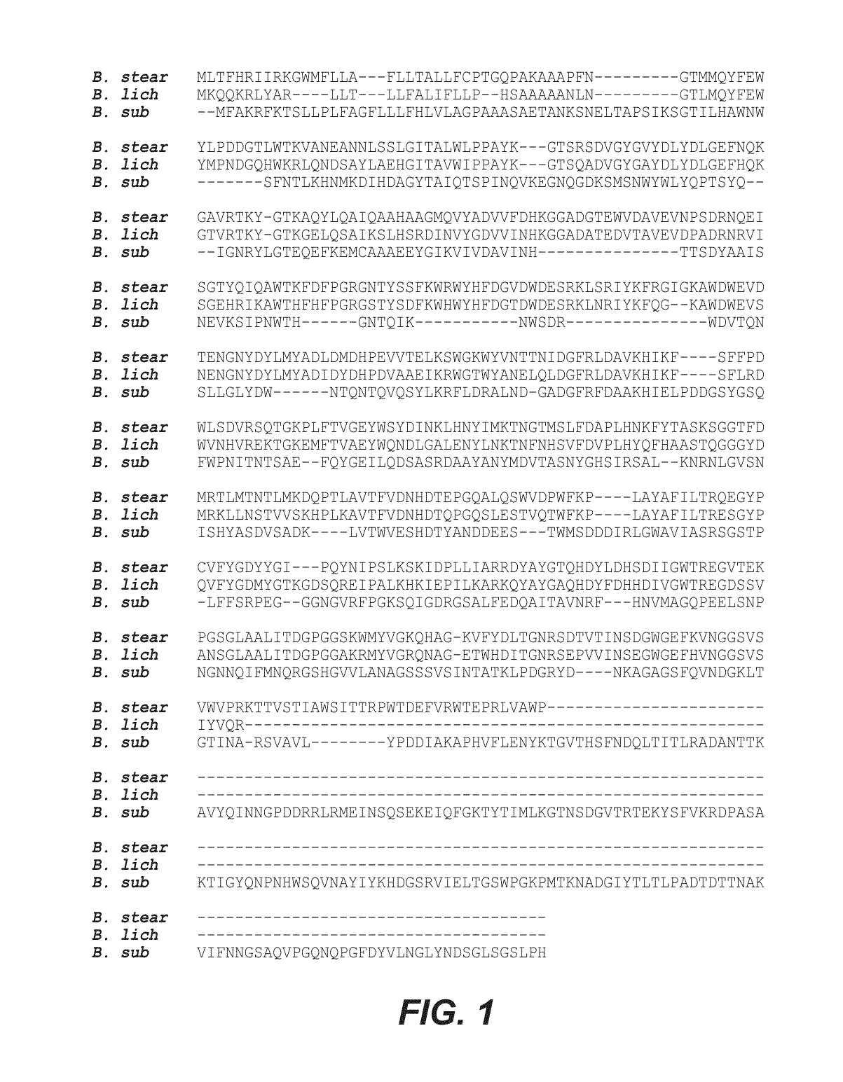

Although alpha-amylases universally contain the three domains discussed above, the three-dimensional structures of some alpha-amylases, such as AmyE from B. subtilis, differ from Termamyl-like alpha-amylases. These enzymes are collectively referred as non-Termamyl-like alpha-amylases. FIG. 1 depicts a sequence alignment of alpha-amylases from Geobacillus stearothermophilus (SEQ ID NO: 25; AmyS), Bacillus licheniformis (SEQ ID NO: 26), and Bacillus subtilis (SEQ ID NO: 27; AmyE). The sequence alignment was generated by the Kalign 2.0 program (available at www.ebi.ac.uk/Tools/kalign/index.html; see also Lassmann & Sonnhammer, BMC Bioinformatics 6: 298 (2005)). The Termamyl-like AmyS and AmyL share approximately 63% identity and approximately 77% similarity, while AmyE shares approximately 15% identity and less than 25% similarity with AmyL or AmyS.

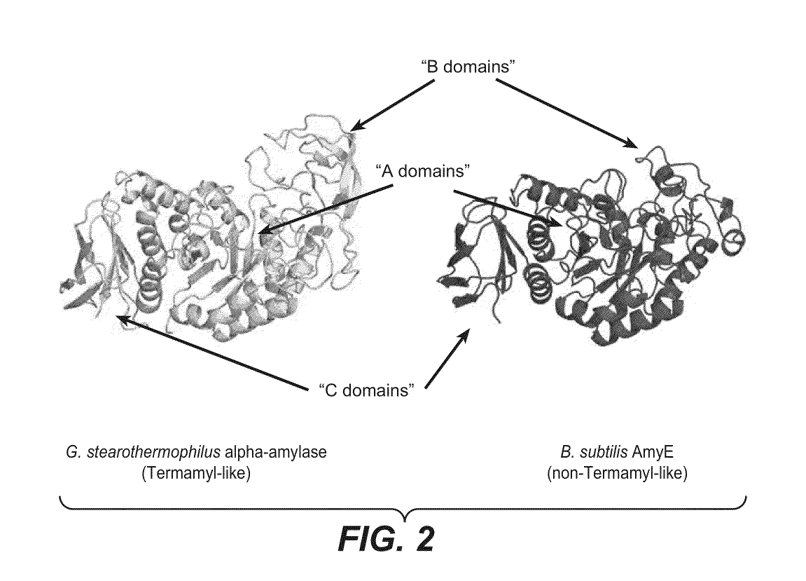

The crystal structure of Bacillus subtilis alpha-amylase (AmyE) or its truncated variant has been determined, and it shares the common features of other alpha-amylases. Fujimoto et al., J. Mol. Biol. 277: 393-407 (1998) (Protein Data Bank Accession No. 1BAG); Kagawa et al., J. Bacteriol. 185: 6981-84 (2001) (Protein Data Bank Accession No. 1UA7). It is of particular interest to compare the crystal structure of AmyE with those “Termamyl-like alpha-amylases.” As indicated in FIG. 2, a common topological scheme can be identified by comparing the three-dimensional structures between AmyE and AmyS. Both amylases display a similar overall structure with three domains. See, e.g., Protein Data Bank Accession Nos. 1UA7 and 1HVX, respectively.

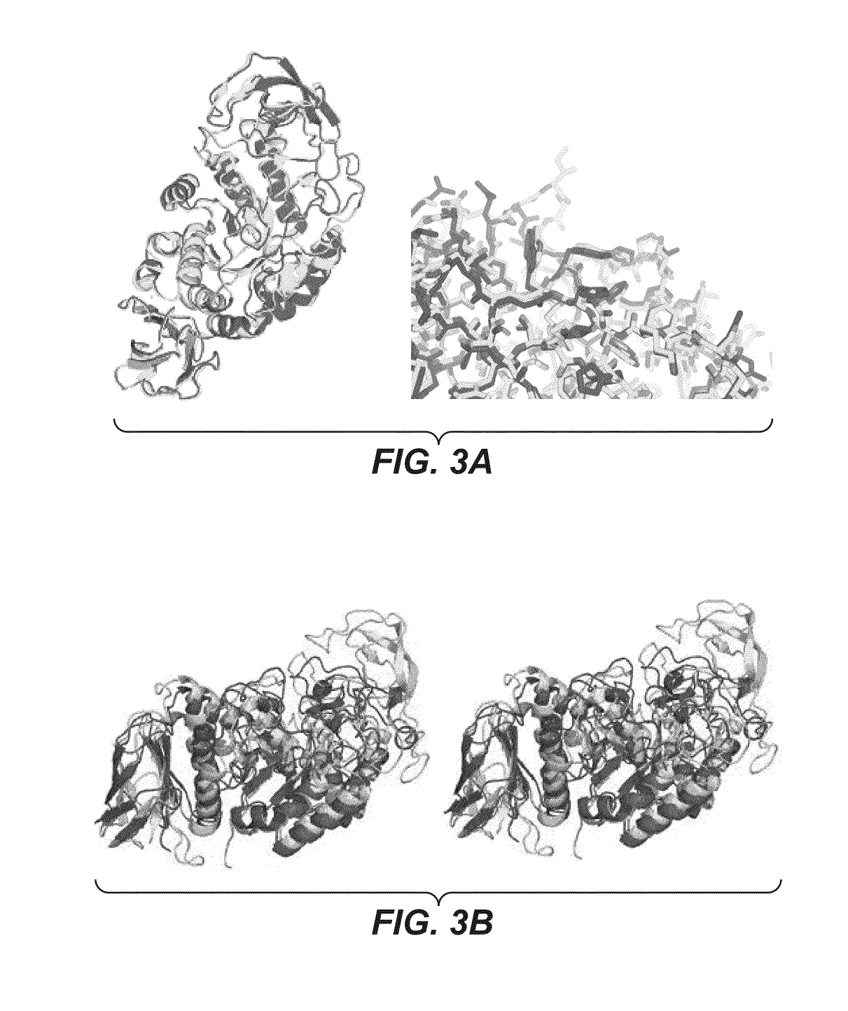

A close examination of the three-dimensional structures of AmyS, AmyL, and AmyE, however, reveals considerable structural difference between AmyE and the Termamyl-like alpha-amylases. When AmyS and AmyL are superposed together, these two amylases almost overlap for each of the three domains. Significant differences are present only at the amino acid side chain level. See FIG. 3A. FIG. 3B, on the other hand, provides superimposed three-dimensional structures of AmyS and AmyE. There are considerable structural differences between AmyS and AmyE. The most dramatic difference can be located in the domain B. Since domain B is commonly believed to form a large portion of the catalytic site, it is expected that AmyE may display enzymatic properties different from those of the Termamyl-like alpha-amylases.

A more quantitative measure for structural similarity is through determining the root mean square deviation (RMSD) based on a given three-dimensional alignment. See Maiorov & Crippen, J. Mol. Biol. 235: 625-634 (1994). RMSD, as the measure of the average distance between the backbones of superimposed proteins, and can be calculated as follows:

$$RMSD = \sqrt{\frac{1}{N} {\sum }_{i = 1}^{N} {δ}_{i}^{2}}$$

where δᵢ represents the distance between the i^{th} pair among the N pairs of equivalent atoms, e.g., alpha-carbon atoms. Typically, one may measure the similarity in three-dimensional structure by the RMSD of the alpha-carbon atomic coordinates after optimal rigid body superposition. When the three-dimensional structure of AmyL (Protein Data Bank Accession No. 1BLI) is superimposed to that of AmyS (Protein Data Bank Accession No. 1HVX), the RMSD is 0.408 angstrom among 419 amino acid residues based on PyMOL (available at www.pymol.org). The three-dimensional structure comparison between AmyE (Protein Data Bank Accession No. 1UA7) and AmyS (Protein Data Bank Accession No. 1HVX), however, generate a RMSD of 8.134 angstroms among 311 amino acid residues.

### 2.2. AmyE and Variants

AmyE enzymes and variants thereof are provided, which are useful for carrying out the applications disclosed herein. Nucleic acids encoding AmyE and variants thereof also are provided, as are vectors and host cells comprising the nucleic acids.

“AmyE” for the purpose of this disclosure means a naturally occurring alpha-amylase (EC 3.2.1.1; 1,4-α-D-glucan glucanohydrolase) from B. subtilis. A representative AmyE sequence is set forth in SEQ ID NO: 1 or 9. The amino acid sequence of AmyE shown in SEQ ID NO: 1 is that of the mature form, without the native signal sequence. The amino acid sequence of AmyE shown in SEQ ID NO: 9 contains a signal sequence consisting of 41 amino acid residues. The amino acid sequence of the native signal sequence of this AmyE is shown in SEQ ID NO: 17. The mature form of this AmyE is referred to elsewhere herein as “AmyE full-length.” Other AmyE sequences have at least about 80%, about 85%, about 90%, about 95%, or about 98% sequence identity to the AmyE of SEQ ID NO: 1, using the BLAST sequence alignment algorithm with default alignment parameters. For example, an AmyE known as Amy31A, disclosed in UniProtKB/TrEMBL Accession No. 082953 (SEQ ID NO: 5), has an 86% sequence identity to the AmyE of SEQ ID NO: 1. The N-terminal 45 amino acid residues of SEQ ID NO: 5 are the signal sequence of Amy31A. AmyE enzymes include, but are not limited to, the AmyE having the amino acid sequence disclosed in NCBI Accession No. ABW75769 (SEQ ID NO: 10). Further AmyE protein sequences include those disclosed in NCBI Accession Nos. ABK54355 (SEQ ID NO: 11), AAF14358 (SEQ ID NO: 12), AAT01440 (SEQ ID NO: 13), AAZ30064 (SEQ ID NO: 14), AAQ83841 (SEQ ID NO: 15), and BAA31528 (SEQ ID NO: 16).

An AmyE “variant” comprises an amino acid sequence modification of a naturally occurring AmyE sequence. As used herein, a naturally occurring AmyE is also a “parent enzyme,” “parent sequence,” “parent polypeptide,” or “wild-type AmyE.” The amino acid modification may comprise an amino acid substitution, addition, or deletion. The amino acid modification in the AmyE variant may be the result of a naturally occurring mutation or the result of deliberate modification of the amino sequence using one of the well-known methods in the art for this purpose, described further below. Representative AmyE variants are disclosed in U.S. patent application Ser. No. 12/479,427, filed Jun. 5, 2009, which is incorporated herein by reference in its entirety.

An AmyE variant, unless otherwise specified, has at least one amino acid modification, but the variant retains at least about 80%, about 85%, about 90%, about 95%, or about 98% amino acid sequence identity to the AmyE of SEQ ID NO: 1, measured by a BLAST alignment of the protein sequences with default alignment parameters. For example, the variant may have one, two, three, up to five, up to ten, or up to 20 amino acid substitutions compared to the amino acid sequence of SEQ ID NO: 1. Typically, modifications are made to amino acid residues that are not required for biological function. The selection of amino acid residues to be modified may be guided by sequence homology among AmyE sequences. Generally, amino acids that are well conserved in AmyE sequences are more likely to be required for biological activity. Conversely, amino acid positions that vary among AmyE sequences are less likely to be required for biological activity.

A variant AmyE may display substantial structural identity to a naturally occurring AmyE within the B domain, e.g., amino acid residues 101-151 of SEQ ID NO: 1. In one embodiment, a variant AmyE may comprises 1-3 amino acid substitutions as to the amino acid residues of the B domain of a naturally occurring AmyE. In another embodiment, a variant AmyE may have a three-dimensional structure that overlaps that of a naturally occurring AmyE, either overall or only the B domain, within 2 angstroms on average.

In some embodiments, a variant AmyE may display one or more altered properties compared to those of the parent enzyme. The altered properties may result in improved performance of the variant compared to its parent. These properties may include substrate specificity, substrate binding, substrate cleavage pattern, thermal stability, pH/activity profile, pH/stability profile, stability towards oxidation, stability at lower levels of calcium ion (Ca²⁺), and/or specific activity.

AmyE or variants thereof may be expressed as a fusion protein that comprises sequences at the N- and/or C-terminus of the mature form of AmyE that facilitate expression, detection, and/or purification, e.g., a signal sequence or a His-tag. Such a sequence includes a signal sequence, which facilitates secretion and expression of the AmyE in a host organism. Additional amino acid residues may be cleaved from the N-terminus of an AmyE, following cleavage of the signal sequence, as discussed in Yang et al., “Nucleotide sequence of the amylase gene from Bacillus subtilis,” Nucleic Acids Res. 11: 237-49 (1983). A “mature form” of an AmyE is defined as the product of all such post-translational modifications of the expressed AmyE sequence. Sequences found at the N-terminus of the primary translation product that are cleaved to form the mature AmyE may be designated alternatively as a “signal sequence,” “leader sequence,” or “pro-sequence.”

The signal sequence may encoded by the same gene as the AmyE. For example, the AmyE set forth in SEQ ID NO: 1 is expressed naturally with a signal sequence and additional N-terminal amino acids having the sequence MFAKRFKTSLLPLFAGFLLLFHLVLAGPAAASAETANKSNE (SEQ ID NO: 17). The signal sequence alternatively may be a B. subtilis sp. signal sequence from a different AmyE or even a different protein. Further, the signal sequence may be from a different species, e.g., B. licheniformis. The signal sequence may be chosen to provide optimal expression of the AmyE or variant thereof in a particular host cell, for example. The mature AmyE may be produced as a result of proteolytic cleavage of additional sequences from the N-terminus that are not signal sequences. For example, a 31-amino acid residue signal sequence from B. licheniformis (“LAT leader sequence”) may be fused in frame with an AmyE sequence.

An AmyE variant for the purpose of this disclosure has at least partial or similar 1,4-α-D-glucan glucanohydrolase activity, compared to a naturally occurring AmyE. Furthermore, an AmyE variant for the purpose of this disclosure may also have a similar level of transglucosidase activity compared to the AmyE having an amino acid sequence of SEQ ID NO: 1. The transglucosidase activity is measured based on the enzymatic synthesis of maltotriose from maltose as described in Example 2. Variants may have the same activity and properties as the naturally-occurring AmyE, or variants may have an altered property, compared to the AmyE having an amino acid sequence of SEQ ID NO: 1. The altered property may be an altered, e.g., two- or three-fold higher, specific activity toward maltoheptaose and/or maltotriose substrates. The thermostability of the protein alternatively or additionally may be altered. For example, the variant may be more thermostable than AmyE. The altered property alternatively or additionally may be the optimal pH for enzymatic activity. For example, the variant may have a more acidic or alkaline optimum pH.

A “truncated” AmyE (“AmyE-tr”) means an AmyE with a sequence deletion of all or part of the C-terminal starch-binding domain. In the AmyE-tr of SEQ ID NO: 3, for example, the AmyE of SEQ ID NO: 1 is truncated at residue D425. A 2.5 Å resolution crystal structure of this AmyE-tr is available at Protein Databank Accession No. 1BAG, which is disclosed in Fujimoto et al., “Crystal structure of a catalytic-site mutant alpha-amylase from B. subtilis complexed with maltopentaose,” J. Mol. Biol. 277: 393-407 (1998). AmyE-tr may be truncated at other positions, e.g., Y423, P424, D426, or 1427 of the AmyE of SEQ ID NO: 1, provided all or part of the C-terminal starch binding domain is removed.

Nucleic acids encoding AmyE or a variant thereof include, but are not limited to, the polynucleotide disclosed in SEQ ID NO: 2 and NO: 4, which encode the AmyE of SEQ ID NO: 1 and AmyE-tr (SEQ ID NO: 3), respectively. Further representative polynucleotides include that disclosed in SEQ ID NO: 6, which encodes Amy31A (SEQ ID NO: 5). The AmyE disclosed in NCBI Accession Nos. ABK54355, AAF14358, AAT01440, AAZ30064, NP_{—}388186, AAQ83841, and BAA31528 likewise are encoded by polynucleotides disclosed in publicly accessible databases, which sequences are incorporated herein by reference. Nucleic acids may be DNA, mRNA, or cDNA sequences. Nucleic acids further include “degenerate sequences” of any of the aforementioned nucleic acids. A degenerate sequence contains at least one codon that encodes the same amino acid residue but has a different nucleotide sequence from the aforementioned nucleic acid sequences. For example, nucleic acids include any nucleic acid sequence that encodes an AmyE or variant thereof. Degenerate sequences may be designed for optimal expression by using codons preferred by a particular host organism.

Vectors comprising the nucleic acids encoding AmyE or variants thereof also are provided. Host cells comprising the vectors are provided. The host cell may express the polynucleotide encoding the AmyE variant. The host may be a Bacillus sp., e.g., B. subtilis.

### 2.3. Characterization of AmyE Variants

AmyE variants can be characterized by their nucleic acid and primary polypeptide sequences, by 3D structural modeling, and/or by their specific activity. Additional characteristics of the AmyE variant include stability, Ca²⁺ dependence, pH range, oxidation stability, and thermostability. In one aspect, the AmyE variants are expressed at higher levels than the wild-type AmyE, while retaining the performance characteristics of the wild-type AmyE. Levels of expression and enzyme activity can be assessed using standard assays known to the artisan skilled in this field. In another aspect, variants demonstrate improved performance characteristics relative to the wild-type enzyme, such as improved stability at high temperatures or improved activity at various pH values, e.g., pH 4.0 to 6.0 or pH 8.0 to 11.0.

The AmyE variant may be expressed at an altered level in a host cell compared to AmyE. Expression generally relates to the amount of active variant that is recoverable from a fermentation broth using standard techniques known in this art over a given amount of time. Expression also can relate to the amount or rate of variant produced within the host cell or secreted by the host cell. Expression also can relate to the rate of translation of the mRNA encoding the variant enzyme.

In a further aspect, some mutations exhibit altered stability or specific activity, especially at temperatures around about 60° C., e.g., about 50° C. to about 70° C., for use in the elimination or reduction of iodine-positive starch and/or treatment of blue saccharide, for example. Variants may have altered stability or specific activity at other temperatures, depending on whether the variant is to be used in other applications or compositions.

AmyE variants also may have altered oxidation stability, in particular higher oxidation stability, in comparison to the parent AmyE. For example, increased oxidation stability is advantageous in detergent compositions, and decreased oxidation stability may be advantageous in composition for starch liquefaction.

The AmyE variants described herein can also have mutations that extend half-life relative to the parent enzyme by about 10%, about 20%, about 30%, about 40%, about 50%, about 60%, about 70%, about 80%, about 90%, about 100%, about 200%, or more, particularly at elevated temperatures of about 55° C. to about 95° C. or more, particularly at about 80° C.

The AmyE variants may have exo-specificity, measured by exo-specificity indices described herein, for example. AmyE variants include those having higher or increased exo-specificity compared to the parent enzymes or polypeptides from which they were derived, optionally when measured under identical conditions. Thus, for example, the AmyE variant polypeptides may have an exo-specificity index of about 10%, about 20%, about 30%, about 40%, about 50%, about 60%, about 70%, about 80%, about 90%, about 100%, about 150%, about 200%, about 500%, about 1000%, about 5000%, about 10,000% or higher compared to their parent polypeptides.

In one aspect, the AmyE variant has the same pH stability as the parental sequence. In another aspect, the variant comprises a mutation that confers a greater pH stability range or shifts the pH range to a desired area for the end commercial purpose of the enzyme. For example, in one embodiment, the variant can degrade starch at about pH 5.0 to about pH 10.5. The AmyE variant polypeptide may have a longer half-life or higher activity (depending on the assay) compared to the parent polypeptide under identical conditions, or the AmyE variant may have the same activity as the parent polypeptide. The AmyE variant polypeptide also may have about 10%, 20%, 30%, 40%, 50%, 60%, 70%, 80%, 90%, 100%, 200% or longer half-life compared to their parent polypeptide under identical pH conditions. Alternatively, or in addition, the AmyE variant may have higher specific activity compared to the parent polypeptide under identical pH conditions.

In another aspect, a nucleic acid complementary to a nucleic acid encoding any of the AmyE variants set forth herein is provided. Additionally, a nucleic acid capable of hybridizing to the complement is provided. In another embodiment, the sequence for use in the methods and compositions described herein is a synthetic sequence. It includes, but is not limited to, sequences made with optimal codon usage for expression in a particular host organism.

### 3. PRODUCTION OF ALPHA-AMYLASES

A DNA sequence encoding the alpha-amylase produced by methods described herein, or by any alternative methods known in the art, can be expressed, in enzyme form, using an expression vector which typically includes control sequences encoding a suitable promoter, operator, ribosome binding site, translation initiation signal, and, optionally, a repressor gene or various activator genes.

### 3.1. Vectors

The recombinant expression vector carrying the DNA sequence encoding the alpha-amylase may be any vector that may conveniently be subjected to recombinant DNA procedures, and the choice of vector will often depend on the host cell into which it is to be introduced. Thus, the vector may be an autonomously replicating vector, i.e., a vector that exists as an extrachromosomal entity, the replication of which is independent of chromosomal replication, e.g., a plasmid, a bacteriophage or an extrachromosomal element, mini-chromosome or an artificial chromosome. Alternatively, the vector may be one which, when introduced into a host cell, is integrated into the host cell genome and replicated together with the chromosome(s) into which it has been integrated. The integrated gene may also be amplified to create multiple copies of the gene in the chromosome by use of an amplifiable construct driven by antibiotic selection or other selective pressure, such as an essential regulatory gene or by complementation of an essential metabolic pathway gene.

An expression vector typically includes the components of a cloning vector, e.g., an element that permits autonomous replication of the vector in the selected host organism and one or more phenotypically detectable markers for selection purposes. The expression vector normally comprises control nucleotide sequences encoding a promoter, operator, ribosome binding site, translation initiation signal and optionally, a repressor gene or one or more activator genes. In one aspect, all the signal sequences used target the material to the cell culture media for easier enzyme collection and optionally purification. The procedures used to ligate the DNA construct encoding a described alpha-amylase, the promoter, the terminator and other elements, respectively, and to insert them into suitable vectors containing the information necessary for replication, are well known to persons skilled in the art (see e.g., Sambrook et al., MOLECULAR CLONING: A LABORATORY MANUAL, 2^{nd} ed., Cold Spring Harbor, 1989 and 3^{rd} ed., 2001).

In the vector, the DNA sequence should be operably connected to a suitable promoter sequence. The promoter may be any DNA sequence that shows transcriptional activity in the host cell of choice and may be derived from genes encoding proteins either homologous or heterologous to the host cell. Suitable promoters for directing the transcription of the DNA sequence encoding an alpha-amylase described herein, especially in a bacterial host, include various Bacillus-derived promoters, such as an alpha-amylase promoter derived from B. subtilis, B. licheniformis, G. stearothermophilus, or B. amyloliquefaciens, the promoter of the lac operon of E. coli, the Streptomyces coelicolor agarase gene dagA or celA promoters, and the promoters of the Bacillus subtilis xylA and xylB genes, etc. For transcription in a fungal host, examples of useful promoters are those derived from the gene encoding Aspergillus oryzae TAKA amylase, Rhizomucor miehei aspartic proteinase, Aspergillus niger neutral alpha-amylase, A. niger acid stable alpha-amylase, A. niger glucoamylase, Rhizomucor miehei lipase, A. oryzae alkaline protease, A. oryzae triose phosphate isomerase, or A. nidulans acetamidase. When the gene encoding an alpha-amylase described herein is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the AOX1 and AOX2 promoters of Pichia pastoris.

The expression vector may also comprise a suitable transcription terminator and, in eukaryotes, polyadenylation sequences operably connected to the DNA sequence encoding the alpha-amylase variant. Termination and polyadenylation sequences may suitably be derived from the same sources as the promoter. The vector may further comprise a DNA sequence enabling the vector to replicate in the host cell in question. Examples of such sequences are the origins of replication of plasmids pUC19, pACYC177, pUB110, pE194, pAMB1, pICatH, and pIJ702.

The vector may also comprise a selectable marker, e.g., a gene the product of which complements a defect in the host cell, such as the dal genes from B. subtilis or B. licheniformis, or a gene that confers antibiotic resistance, e.g., ampicillin, kanamycin, chloramphenicol, or tetracycline resistance. Furthermore, the vector may comprise Aspergillus selection markers such as amdS, argB, niaD, and xxsC, a marker giving rise to hygromycin resistance, or the selection may be accomplished by co-transformation as known in the art. See, e.g., WO 91/17243.

### 3.2 Variant Expression and Host Organisms

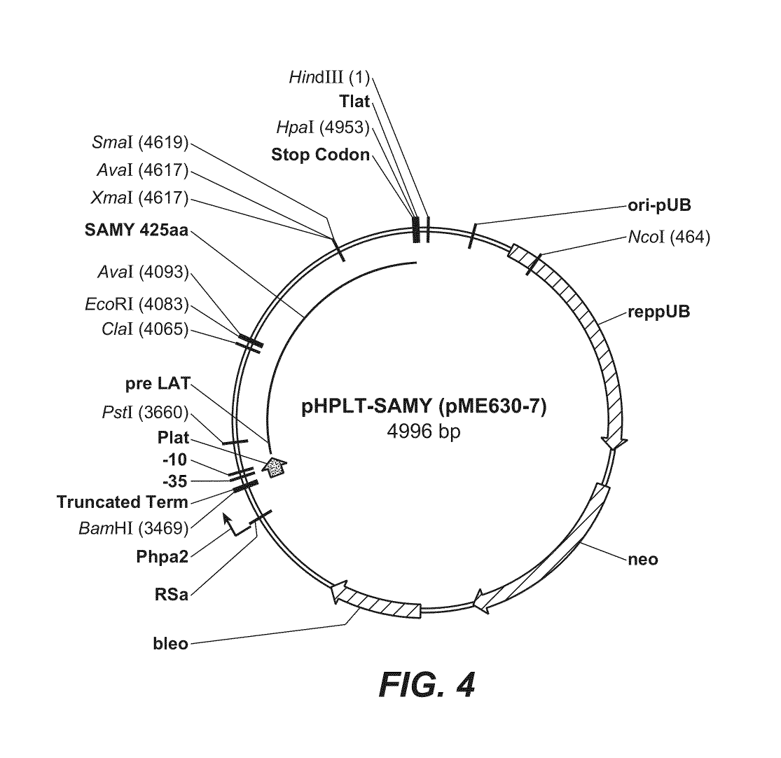

It is generally advantageous if the alpha-amylase is secreted into the culture medium, when expressed in a host cell. To this end, the alpha-amylase may comprise a signal sequence that permits secretion of the expressed enzyme into the culture medium. If desirable, this original signal sequence may be replaced by a different signal sequence, which is conveniently accomplished by substitution of the DNA sequences encoding the respective signal sequence. For example, a nucleic acid encoding AmyE is operably linked to a B. licheniformis signal sequence in the expression vector shown in FIG. 4. Signal sequences are discussed in more detail above.

An isolated cell, comprising either a DNA construct or an expression vector, can be used as a host cell in the recombinant production of the alpha-amylase. The cell may be transformed with the DNA construct encoding the alpha-amylase, optionally by integrating the DNA construct (in one or more copies) in the host chromosome. This integration is generally considered to be an advantage as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram-positive bacterial species such as Bacillaceae, including B. subtilis, B. licheniformis, B. lentus, B. brevis, B. stearothermophilus, B. alkalophilus, B. amyloliquefaciens, B. coagulans, B. lautus, B. megaterium, and B. thuringiensis; Streptomyces sp., such as S. murinus; lactic acid bacterial species including Lactococcus sp., such as L. lactis; Lactobacillus sp., including L. reuteri; Leuconostoc sp.; Pediococcus sp.; and Streptococcus sp. Still other useful hosts include Bacillus sp. A 7-7, for example. Alternatively, strains of a Gram-negative bacterial species belonging to Enterobacteriaceae, including E. coli, or to Pseudomonadaceae can be selected as the host organism.

A suitable yeast host organism can be selected from biotechnologically relevant yeasts species, such as, but not limited to, Pichia sp., Hansenula sp., Kluyveromyces sp., Yarrowinia sp., Saccharomyces sp., including S. cerevisiae, or a species belonging to Schizosaccharomyces, such as S. pombe. A strain of the methylotrophic yeast species Pichia pastoris can be used as the host organism. Alternatively, the host organism can be a Hansenula species. Suitable host organisms among filamentous fungi include species of Aspergillus, e.g., A. niger, A. oryzae, A. tubigensis, A. awamori, or A. nidulans. Alternatively, a strain of Fusarium sp., e.g., Fusarium oxysporum or Rhizomucor sp., such as R. miehei, can be used as the host organism. Other suitable yeasts include Thermomyces sp. and Mucor sp. Fungal cells may be transformed by a process involving protoplast formation and transformation of the protoplasts followed by regeneration of the cell wall in a manner known in the art. An exemplary procedure for transforming Aspergillus host cells, for example, is described in EP 238023.

In a yet further aspect, a method of producing an alpha-amylase is provided, which method comprises cultivating a host cell as described above under conditions conducive to the production of the variant and recovering the variant from the cells and/or culture medium. The medium used to cultivate the cells may be any conventional medium suitable for growing the host cell in question and obtaining expression of the alpha-amylase. Suitable media and media components are available from commercial suppliers or may be prepared according to published recipes, e.g., as described in catalogues of the American Type Culture Collection (ATCC). Exemplary culture media include, but are not limited to, those for fed-batch fermentations performed in a three thousand liter (3,000 L) stirred tank fermentor. The growth medium in that case can consist of corn steep solids and soy flour as sources of organic compounds, along with inorganic salts as a source of sodium, potassium, phosphate, magnesium and sulfate, as well as trace elements. Typically, a carbohydrate source such as glucose is also part of the initial medium. Once the culture has established itself and begins growing, the carbohydrate is metered into the tank to maintain the culture as is known in the art. Samples are removed from the fermentor at regular intervals to measure enzyme titer using, for example, a colorimetric assay method. The fermentation process is halted when the enzyme production rate stops increasing according to the measurements.

The alpha-amylase secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

Host cells may be cultured under suitable conditions that allow expression of the alpha-amylase. Expression of the proteins may be constitutive such that they are continually produced, or inducible, requiring a stimulus to initiate expression. In the case of inducible expression, protein production can be initiated when required by addition of an inducer substance, e.g., dexamethasone, IPTG, or Sepharose, to the culture medium, for example. Polypeptides can also be produced recombinantly in an in vitro cell-free system, such as the TnT™ (Promega) rabbit reticulocyte system.

A host for expressing the alpha-amylase can be cultured under aerobic conditions in the appropriate medium for the host. Shaking or a combination of agitation and aeration can be provided, with production occurring at the appropriate temperature for that host, e.g., from about 30° C. to about 75° C., depending on the needs of the host and production of the desired alpha-amylase variant. Culturing can occur from about 12 to about 100 hours or greater (and any hour value there between) or more particularly from about 24 to 72 hours. Typically, the culture broth is at a pH of about 5.5 to about 8.0, again depending on the culture conditions needed for the host cell relative to production of the desired alpha-amylase.

The amylolytic activity of the expressed enzyme may be determined using potato starch as substrate, for example. This method is based on the break-down of modified potato starch by the enzyme, and the reaction is followed by mixing samples of the starch/enzyme solution with an iodine solution. Initially, a blackish-blue color is formed, but during the break-down of the starch, the blue color gets weaker and gradually turns into a reddish-brown, which is compared to a colored glass standard.

### 4. PURIFICATION OF THE ALPHA-AMYLASE

Conventional methods can be used in order to prepare a purified alpha-amylase described herein. After fermentation, a fermentation broth is obtained, and the microbial cells and various suspended solids, including residual raw fermentation materials, are removed by conventional separation techniques to obtain an amylase solution. Filtration, centrifugation, microfiltration, rotary vacuum drum filtration, followed by ultra-filtration, extraction or chromatography, or the like are generally used.

It is desirable to concentrate the solution containing the expressed alpha-amylase described herein to optimize recovery, since the use of un-concentrated solutions may require increased incubation time to collect precipitates containing the purified enzyme. The solution is concentrated using conventional techniques until the desired enzyme level is obtained. Concentration of the enzyme containing solution may be achieved by any of the techniques discussed above. In one embodiment, rotary vacuum evaporation and/or ultrafiltration is used. Alternatively, ultrafiltration can be used.

By “precipitation agent” for purposes of purification is meant a compound effective to precipitate the alpha-amylase described herein from solution, whatever the nature of the precipitate may be, i.e., crystalline, amorphous, or a blend of both. Precipitation can be performed using, for example, a metal halide precipitation agent. Metal halide precipitation agents include: alkali metal chlorides, alkali metal bromides and blends of two or more of these metal halides. The metal halide may be selected from the group consisting of sodium chloride, potassium chloride, sodium bromide, potassium bromide and blends of two or more of these metal halides. Suitable metal halides include sodium chloride and potassium chloride, particularly sodium chloride, which can further be used as a preservative. The selection of conditions of the precipitation for maximum recovery, including incubation time, pH, temperature and concentration of an alpha-amylase described herein, will be readily apparent to one of ordinary skill in the art after routine testing.

Generally, at least about 5% w/v (weight/volume) to about 25% w/v of metal halide is added to the concentrated enzyme variant solution, and usually at least about 8% w/v. Generally, no more than about 25% w/v of metal halide is added to the concentrated enzyme variant solution and usually no more than about 20% w/v. The optimal concentration of the metal halide precipitation agent will depend, among others, on the nature of the specific alpha-amylase described herein and on its concentration in solution.

Another alternative to effect precipitation of the enzyme is to use of organic compounds, which can be added to the concentrated enzyme variant solution. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of said organic compound precipitation agents can take place prior to, simultaneously with or subsequent to the addition of the metal halide precipitation agent, and the addition of both precipitation agents, organic compound and metal halide, may be carried out sequentially or simultaneously. For further descriptions, see, e.g., U.S. Pat. No. 5,281,526 to Danisco US, Inc., Genencor Division, for example.

Generally, the organic compound precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. The organic compound precipitations agents can be for example linear or branched alkyl esters of 4-hydroxybenzoic acid, wherein the alkyl group contains from 1 to 10 carbon atoms, and blends of two or more of these organic compounds. Suitable organic compounds include linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl ester of 4-hydroxybenzoic acid, butyl ester of 4-hydroxybenzoic acid, ethyl ester of 4-hydroxybenzoic acid and blends of two or more of these organic compounds can also be used. Additional organic compounds also include, but are not limited to, 4-hydroxybenzoic acid methyl ester (methyl PARABEN) and 4-hydroxybenzoic acid propyl ester (propyl PARABEN), which are also amylase preservative agents. Addition of such an organic compound precipitation agent provides the advantage of high flexibility of the precipitation conditions with respect to pH, temperature, enzyme concentration, precipitation agent concentration, and time of incubation. Generally, at least 0.01% w/v of organic compound precipitation agent is added to the concentrated enzyme variant solution and usually at least 0.02% w/v. Generally, no more than 0.3% w/v of organic compound precipitation agent is added to the concentrated enzyme variant solution and usually no more than 0.2% w/v.

The concentrated enzyme solution, containing the metal halide precipitation agent and, in one aspect, the organic compound precipitation agent, is adjusted to a pH that may depend on the enzyme variant to be purified. Generally, the pH is adjusted to a level near the isoelectric point (pI) of the amylase. For example, the pH can be adjusted within a range of about 2.5 pH units below the pI to about 2.5 pH units above the pI. The pH may be adjusted accordingly if the pI of the variant differs from the wild-type pI.

The incubation time necessary to obtain a purified enzyme precipitate depends on the nature of the specific enzyme, the concentration of enzyme, and the specific precipitation agent(s) and its (their) concentration. Generally, the time effective to precipitate the enzyme variant is between about 1 to about 30 hours; usually it does not exceed about 25 hours. In the presence of the organic compound precipitation agent, the time of incubation can still be reduced to less than about 10 hours, and in most cases even about 6 hours.

Generally, the temperature during incubation is between about 4° C. and about 50° C. Usually, the method is carried out at a temperature between about 10° C. and about 45° C., and particularly between about 20° C. and about 40° C. The optimal temperature for inducing precipitation varies according to the solution conditions and the enzyme or precipitation agent(s) used.

The overall recovery of purified enzyme precipitate, and the efficiency with which the process is conducted, is improved by agitating the solution comprising the enzyme, the added metal halide and the added organic compound. The agitation step is done both during addition of the metal halide and the organic compound, and during the subsequent incubation period. Suitable agitation methods include mechanical stirring or shaking, vigorous aeration, or any similar technique.

The purified enzyme may be further purified by conventional separation techniques, such as filtration, centrifugation, microfiltration, rotary vacuum filtration, ultrafiltration, press filtration, cross membrane microfiltration, cross flow membrane microfiltration, or the like. Cross membrane microfiltration can be one method used. Further purification of the purified enzyme precipitate can be obtained by washing the precipitate with water. For example, the purified enzyme precipitate may be washed with water containing the metal halide precipitation agent, for example, with water containing the metal halide and the organic compound precipitation agents.

During culturing, expressed enzyme may accumulate in the culture broth. For the isolation and purification of the expressed enzyme, the culture broth may be centrifuged or filtered to eliminate cells, and the resulting cell-free liquid may be used for the purification of the enzyme. In one embodiment, the cell-free broth is subjected to salting out using ammonium sulfate at about 70% saturation; the 70% saturation-precipitation fraction is then dissolved in a buffer and applied to a column such as a Sephadex G-100 column, and eluted to recover the enzyme active fraction. For further purification, a conventional procedure such as ion exchange chromatography may be used.

Purified enzymes are useful for all applications in which the enzymes are generally utilized. For example, they can be used in laundry detergents and spot removers, in the food industry, in starch processing and baking, and in pharmaceutical compositions as digestive aids. They can be made into a final product that is either liquid (solution, slurry) or solid (granular, powder).

Alternatively, the enzyme product can be recovered and a flocculating agent is added to the media in order to remove cells and cell debris by filtration or centrifugation without further purification of the enzyme.

The alpha-amylase that is produced and purified by the methods described above can be used in a variety of useful industrial applications. The enzymes possess valuable properties facilitating applications related to fabric and household care (F&HC). For example, an alpha-amylase described herein can be used as a component in washing, dishwashing and hard-surface cleaning detergent compositions. Alpha-amylases described herein also are useful in the production of sweeteners and ethanol from starch, and/or for textile desizing. The described alpha-amylases are particularly useful in starch-conversion processes, including starch liquefaction and/or saccharification processes, as described, for example, in WO 2005/111203 and U.S. Published Application No. 2006/0014265, published Jan. 19, 2006 (Danisco US, Inc., Genencor Division). These uses of described alpha-amylases are described in more detail below.

### 5. APPLICATIONS OF THE ALPHA-AMYLASES IN STARCH PROCESSING

### 5.1. Liquefaction and Saccharification

In one aspect, compositions with the alpha-amylase can be utilized for starch processing, for example, liquefaction and/or saccharification. The process may comprise hydrolysis of a slurry of gelatinized or granular starch, in particular hydrolysis of granular starch into a soluble starch hydrolysate at a temperature below the initial gelatinization temperature of the granular starch. Starch processing is useful for producing sweetener, producing alcohol for fuel or drinking (i.e., potable alcohol), producing a beverage, processing cane sugar, or producing desired organic compounds, e.g., citric acid, itaconic acid, lactic acid, gluconic acid, ketones, amino acids, antibiotics, enzymes, vitamins, and hormones. Conversion of starch to fructose syrups normally consists of three consecutive enzymatic processes: a liquefaction process, a saccharification process, and an isomerization process.

As used herein, the term “liquefaction” or “liquefy” means a process by which starch is converted to less viscous and shorter chain dextrins. Generally, this process involves gelatinization of starch simultaneously with or followed by the addition of an alpha-amylase described herein. As used herein, the term “primary liquefaction” refers to a step of liquefaction when the slurry's temperature is raised to or near its gelatinization temperature. Subsequent to the raising of the temperature, the slurry is sent through a heat exchanger or jet to temperatures from about 90-150° C., e.g., about 100-110° C. Subsequent to application to a heat exchange or jet temperature, the slurry is held for a period of about 3-10 minutes at that temperature. This step of holding the slurry at about 90-150° C. is termed primary liquefaction.

As used herein, the term “secondary liquefaction” refers the liquefaction step subsequent to primary liquefaction (heating to about 90-150° C.), when the slurry is allowed to cool to room temperature. This cooling step can be about 30 minutes to about 180 minutes, e.g. about 90 minutes to about 120 minutes. As used herein, the term “minutes of secondary liquefaction” refers to the time that has elapsed from the start of secondary liquefaction to the time that the Dextrose Equivalent (DE) is measured.

After the liquefaction process, the dextrins typically may be converted into dextrose by addition of a glucoamylase (e.g., AMG™ from Novozymes, A/S) and optionally a debranching enzyme, such as an isoamylase or a pullulanase (e.g., Promozyme® from Novozymes, A/S). Before this step, the pH typically is reduced to a value below about 4.5, while maintaining the temperature at about 95° C. or more, so that the liquefying alpha-amylase variant activity is denatured. The temperature then is lowered to about 60° C., and a glucoamylase and a debranching enzyme are added. The saccharification process proceeds typically for about 24 to about 72 hours.

An advantage of alpha-amylase described herein is their ability to catalyze the breakdown of complex sugars, such as maltose, maltotriose, and maltoheptaose. For this reason, saccharification can be catalyzed by AmyE or a variant thereof with a glucoamylase. A further advantage of the alpha-amylases described herein is that dextrins may be converted into dextrose by the action or one or more alpha-amylases described herein under the same reaction conditions that are optimal for glucoamylase. This advantageous property of AmyE and variants thereof is disclosed in U.S. Provisional Application 61/059,618, filed Jun. 6, 2008, incorporated herein by reference in its entirety. Because AmyE and variants thereof often times operate at the same pH and temperature as glucoamylase, AmyE and variants thereof may be added before or after additional catalysis with a glucoamylase, or by a cocktail of AmyE or a variant thereof and a glucoamylase. The delays necessitated by adjusting the pH and temperature of the reaction to accommodate the use of a glucoamylase thus are avoided.

Glucoamylases, when used alone in saccharification, typically are present in an amount of no more than, or even less than, about 0.5 glucoamylase activity unit (GAU)/g DS (i.e., glucoamylase activity units per gram of dry solids). Glucoamylases may be added in an amount of about 0.02-2.0 GAU/g DS or about 0.1-1.0 GAU/g DS, e.g., about 0.2 GAU/g DS. Glucoamylases are derived from a microorganism or a plant. For example, glucoamylases can be of fungal or bacterial origin. Exemplary bacterial glucoamylases are Aspergillus glucoamylases, in particular A. niger G1 or G2 glucoamylase (Boel et al., EMBO J. 3(5): 1097-1102 (1984)), or variants thereof, such as disclosed in WO 92/00381 and WO 00/04136; A. awamori glucoamylase (WO 84/02921); A. oryzae glucoamylase (Hata et al., Agric. Biol. Chem. 55(4): 941-949 (1991)), or variants or fragments thereof. In one embodiment, the process also comprises the use of a carbohydrate-binding domain of the type disclosed in WO 98/22613. Other contemplated Aspergillus glucoamylase variants include variants to enhance the thermal stability: G137A and G139A (Chen et al., Prot. Eng. 9: 499-505 (1996)); D257E and D293E/Q (Chen et al., Prot. Eng. 8: 575-582 (1995)); N182 (Chen et al., Biochem. J. 301: 275-281 (1994)); disulphide bonds, A246C (Fierobe et al., Biochemistry, 35: 8698-8704 (1996)); and introduction of Pro residues in positions A435 and 5436 (Li et al., Protein Eng. 10: 1199-1204 (1997)). Other contemplated glucoamylases include Talaromyces glucoamylases, in particular derived from T. emersonii (WO 99/28448), T. leycettanus (U.S. Pat. No. RE 32,153), T. duponti, or T. thermophilus (U.S. Pat. No. 4,587,215). Contemplated bacterial glucoamylases include glucoamylases from the genus Clostridium, in particular C. thermoamylolyticum (EP 135138) and C. thermohydrosulfuricum (WO 86/01831). Suitable glucoamylases include the glucoamylases derived from Aspergillus oryzae, such as a glucoamylase having about 50%, about 55%, about 60%, about 65%, about 70%, about 75%, about 80%, about 85%, or even about 90% identity to the amino acid sequence shown in SEQ ID NO: 2 in WO 00/04136. Also suitable are commercial glucoamylases, such as AMG 200L; AMG 300 L; SAN™ SUPER and AMG™ E (Novozymes); OPTIDEX® 300 (Danisco US, Inc., Genencor Division); AMIGASE™ and AMIGASE™ PLUS (DSM); G-ZYME® G900 (Enzyme Bio-Systems); and G-ZYME® G990 ZR (A. niger glucoamylase and low protease content).

Alpha-amylases described herein can be advantageously combined with a glucoamylase in a composition for process starch, e.g., as a composition for saccharification. Because of the advantageous properties of AmyE or its variants thereof, a reduced amount of glucoamylase, for example, about 50%, about 45%, about 40%, about 35%, about 30%, about 25%, about 20%, about 15%, or about 10% less, may be sufficient to achieve an equivalent saccharification result as using glucoamylase alone.

In another embodiment, other alpha- or beta-amylases, or other enzymes to provide a “cocktail” with a broad spectrum of activity. For example, the starch may be contacted with one or more enzyme selected from the group consisting of a fungal alpha-amylase (EC 3.2.1.1), a bacterial alpha-amylase, e.g., a Bacillus alpha-amylase or a non-Bacillus alpha-amylase, and/or a beta-amylase (EC 3.2.1.2). In an embodiment further another amylolytic enzyme or a debranching enzyme, such as an isoamylase (EC 3.2.1.68), or a pullulanases (EC 3.2.1.41) may be added to the alpha-amylase described herein. Isoamylase hydrolyses α-1,6-D-glucosidic branch linkages in amylopectin and β-limit dextrins and can be distinguished from pullulanases by the inability of isoamylase to attack pullulan and by the limited action of isoamylase on α-limit dextrins. Debranching enzymes may be added in effective amounts well known to the person skilled in the art.

Phytases are useful for the present disclosure as they are capable of hydrolyzing phytic acid under the defined conditions of the incubation and liquefaction steps. In some embodiments, the phytase is capable of liberating at least one inorganic phosphate from an inositol hexaphosphate (phytic acid). Phytases can be grouped according to their preference for a specific position of the phosphate ester group on the phytate molecule at which hydrolysis is initiated (e.g., as 3-phytases (EC 3.1.3.8) or as 6-phytases (EC 3.1.3.26)). A typical example of phytase is myo-inositol-hexakisphosphate-3-phosphohydrolase.

Phytases can be obtained from microorganisms such as fungal and/or bacterial organisms. Some of these microorganisms include e.g., Aspergillus (e.g., A. niger, A. terreus, A. ficum and A. fumigatus), Myceliophthora (M. thermophila), Talaromyces (T. thermophilus) Trichoderma spp. (T. reesei). and Thermomyces (WO 99/49740). Phytases are also available from Penicillium species, e.g., P. hordei (ATCC No. 22053), P. piceum (ATCC No. 10519), or P. brevi-compactum (ATCC No. 48944). See, e.g., U.S. Pat. No. 6,475,762. In addition, phytases are available from Bacillus (e.g., B. subtilis), Pseudomonas, Peniophora, E. coli, Citrobacter, Enterbacter, and Buttiauxella (see WO2006/043178)).

Commercial phytases are available such as NATUPHOS (BASF), RONOZYME P (Novozymes A/S), PHZYME XP (Danisco A/S), and FINASE (AB Enzymes). The method for determining microbial phytase activity and the definition of a phytase unit has been published by Engelen et al., J. of AOAC Int., 77: 760-764 (1994). The phytase may be a wild-type phytase, a variant, or a fragment thereof.

In one embodiment, the phytase is one derived from the bacterium Buttiauxiella spp. The Buttiauxiella spp. includes B. agrestis, B. brennerae, B. ferragutiase, B. gaviniae, B. izardii, B. noackiae, and B. warmboldiae. Strains of Buttiauxella species are available from DSMZ, the German National Resource Center for Biological Material (Inhoffenstrabe 7B, 38124 Braunschweig, Germany). Buttiauxella sp. strain P1-29 deposited under accession number NCIMB 41248 is an example of a particularly useful strain from which a phytase may be obtained and used according to the present disclosure. In some embodiments, the phytase is BP-wild-type, a variant thereof (such as BP-11) disclosed in WO 06/043178, or a variant as disclosed in US 2008/0220498, published Sep. 11, 2008. For example, a BP-wild-type and variants thereof are disclosed in Table 1 of WO 06/043178, wherein the numbering is in reference to SEQ ID NO: 3 of the published PCT application.

Beta-amylases are exo-acting maltogenic amylases, which catalyze the hydrolysis of 1,4-α-glucosidic linkages into amylose, amylopectin, and related glucose polymers, thereby releasing maltose. Beta-amylases have been isolated from various plants and microorganisms (Fogarty et al., PROGRESS IN INDUSTRIAL MICROBIOLOGY, Vol. 15, pp. 112-115, 1979). These beta-amylases are characterized by having optimum temperatures in the range from 40° C. to 65° C., and optimum pH in the range from about 4.5 to about 7.0. Contemplated beta-amylases include, but are not limited to, beta-amylases from barley SPEZYME® BBA 1500, SPEZYME® DBA, Optimalt™ ME, Optimalt™ BBA (Danisco A/S); and Novozym™ WBA (Novozymes A/S).

After the saccharification process, the dextrose syrup may be converted into high fructose syrup using an immobilized glucose isomerase (such as Sweetzyme®), for example. In one regard, the soluble starch hydrolysate of the process is subjected to conversion into high fructose starch-based syrup (HFSS), such as high fructose corn syrup (HFCS). This conversion can be achieved using a glucose isomerase, particularly a glucose isomerase immobilized on a solid support. Contemplated isomerases included the commercial products Sweetzyme® IT (Novozymes A/S); G-ZYME® IMGI, and G-ZYME® G993, Ketomax®, G-ZYME® G993 liquid, and GenSweet® IGI (Danisco US Inc., Genencor Division).

While addition of 1 mM Ca²⁺ or more is typically required to ensure adequately high stability of the alpha-amylase, the free Ca²⁺ strongly inhibits the activity of the glucose isomerase. The Ca²⁺ is thus typically removed prior to isomerization, by means of an expensive unit operation, so that the level of free Ca²⁺ concentration is below 3-5 ppm. Cost savings could be obtained if such an operation were avoided.

Alpha-amylases described herein advantageously require less or no added Ca²⁺ for stability. For this reason, the Ca²⁺ added to a liquefaction and/or saccharification reaction may be reduced or eliminated altogether. The removal of Ca²⁺ by ion exchange prior to contacting the reaction mixture with glucose isomerase thus may be avoided, saving time and cost and increasing the efficiency of a process of producing a high fructose syrup.

The starch to be processed may be obtained from tubers, roots, stems, legumes, cereals or whole grain. More specifically, the granular starch may be obtained from corns, cobs, wheat, barley, rye, milo, sago, cassava, tapioca, sorghum, rice, peas, bean, banana, or potatoes. Exemplary starches contemplated are both waxy and non-waxy types of corn and barley. The starch may be a highly refined starch quality, for instance, at least 90%, at least 95%, at least 97%, or at least 99.5% pure. Alternatively, the starch can be a more crude starch containing material comprising milled whole grain, including non-starch fractions such as germ residues and fibers. The raw material, such as whole grain, is milled to open up the structure and allow further processing.

Two milling processes are suitable: wet and dry milling. In dry milling, the whole kernel is milled and used. Wet milling gives a good separation of germ and meal (starch granules and protein) and is usually used in the production of syrups. Both dry and wet milling are well known in the art of starch processing and also are contemplated for use with the compositions and methods disclosed. The process may be conducted in an ultrafiltration system where the retentate is held under recirculation in presence of enzymes, raw starch and water, where the permeate is the soluble starch hydrolysate. Another method is the process conducted in a continuous membrane reactor with ultrafiltration membranes, where the retentate is held under recirculation in presence of enzymes, raw starch and water, and where the permeate is the soluble starch hydrolysate. Also contemplated is the process conducted in a continuous membrane reactor with microfiltration membranes and where the retentate is held under recirculation in presence of enzymes, raw starch and water, and where the permeate is the soluble starch hydrolysate.

Dry milled grain can comprise significant amounts of non-starch carbohydrate compounds, in addition to starch. When such a heterogeneous material is processed by jet cooking, often only a partial gelatinization of the starch is achieved. Accordingly, the described alpha-amylases having a high activity towards ungelatinized starch are advantageously applied in a process comprising liquefaction and/or saccharification jet cooked dry milled starch.

The starch slurry to be used in any of the above aspects may have about 20% to about 55% dry solids granular starch, about 25% to about 40% dry solids granular starch, or about 30% to about 35% dry solids granular starch. The enzyme variant converts the soluble starch into a soluble starch hydrolysate of the granular starch in the amount of at least about 85%, about 86%, about 87%, about 88%, about 89%, about 90%, about 91%, about 92%, about 93%, about 94%, about 95%, about 96%, about 97%, about 98%, or about 99%.

In another embodiment, an alpha-amylase described herein is used in starch processing further comprising fermentation to produce a fermentation product, e.g., ethanol. Such a process for producing ethanol from starch-containing material by fermentation comprises: (i) liquefying the starch-containing material; (ii) saccharifying the liquefied mash obtained; and (iii) fermenting the material obtained in step (ii) in the presence of a fermenting organism. Optionally the process further comprises recovery of the ethanol. During the fermentation, the ethanol content reaches at least about 7%, at least about 8%, at least about 9%, at least about 10% such as at least about 11%, at least about 12%, at least about 13%, at least about 14%, at least 15%, or at least 16% ethanol.

The saccharification and fermentation processes may be carried out as a simultaneous saccharification and fermentation (SSF) process. When fermentation is performed simultaneously with the hydrolysis, the temperature can be between about 30° C. and about 35° C., particularly between about 31° C. and about 34° C. The process may be conducted in an ultrafiltration system where the retentate is held under recirculation in presence of enzymes, raw starch, yeast, yeast nutrients and water and where the permeate can be an ethanol containing liquid. Also contemplated is the process conducted in a continuous membrane reactor with ultrafiltration membranes and where the retentate is held under recirculation in presence of enzymes, raw starch, yeast, yeast nutrients and water and where the permeate is an ethanol containing liquid.

The soluble starch hydrolysate of the process may also be used for production of a fermentation product comprising fermenting the treated starch into a fermentation product, such as citric acid, monosodium glutamate, gluconic acid, sodium gluconate, calcium gluconate, potassium gluconate, glucono delta-lactone, or sodium erythorbate.

### 5.2. Removal of IPS from Saccharide liquor

Industrial starch-processing facilities occasionally encounter process excursions, e.g., temperature, pH, or enzyme dose, any of which may result in the presence of a significant amount of iodine-positive starch (IPS) in a saccharide liquor. Additionally, the present of IPS in a saccharide liquor may result from a poor liquefaction, in which starch is not effectively hydrolyzed. IPS, which comes from amylose that escapes hydrolysis and/or retrograded starch polymer, is able to react with iodine to produce a characteristic blue/purple color. IPS-containing saccharide liquor is thus called blue saccharide. The presence of IPS in saccharide liquor negatively affects final product quality and represents a major issue with downstream processing. The supplementation of AmyE or its variant thereof with a glucoamylase in the saccharification has been shown to reduce the amount of IPS in a statistically significant fashion. Alternatively, the presence of IPS can be remedied, post-saccharification or upon identification, by isolating the saccharification tank and blending the contents back at a level that is undetectable. Although such a remedy is able to reduce the IPS level below the detection by customers, the offending material is still there and will accumulate in carbon columns and filter systems, among other things.

As described herein, AmyE or its variant thereof, may be added to a saccharide liquor to eliminate or reduce the IPS. It thus represents a novel enzymatic solution that enables effective elimination or reduction of IPS upon the detection of IPS post-saccharification. In another embodiment, a phytase can be supplemented with the alpha-amylase to eliminate or reduce the IPS.

The enzyme used may be AmyE, its variant thereof, or an alpha-amylase sharing an amino acid sequence identity of at least about 80%, about 85%, about 90%, about 95%, about 98%, about 99%, or about 99.5% to SEQ ID NO: 1. In one embodiment, the alpha-amylase may comprise 1-3 amino acid substitutions as to the amino acid residues of the B domain of a naturally occurring AmyE. In another embodiment, a variant AmyE may have a three-dimensional structure that overlaps that of a naturally occurring AmyE, either overall or only the B domain, within 2 angstroms on average. The alpha-amylase may display a transglucosidase activity that is at a similar level as that of AmyE having an amino acid sequence of SEQ ID NO: 1.

In one embodiment, the alpha-amylase is used at an amount in the range of about 0.1 to 0.4 mg per gram of starch (mg/g starch), e.g., about 0.1, about 0.15, about 0.2, about 0.25, about 0.3, about 0.35, or about 0.4 mg/g starch to eliminate or remove the IPS in a saccharide liquor. The treatment may be performed at a pH in the range of about 5.0 to about 5.5, e.g., about 5.0, about 5.1, about 5.2, about 5.3, about 5.4, or about 5.5; at a temperature about 58-62° C., e.g., about 60° C.; and for about 4 to about 24 hours, e.g., about 4, about 5, about 6, about 7, about 8, about 9, about 10, about 11, about 12, about 13, about 14, about 15, about 16, about 17, about 18, about 19, about 20, about 21, about 22, about 23, or about 24 hours.

### 5.3. Ethanol Production from Starch

In general, alcohol production (ethanol) from whole grain generally can be separated into four main steps: milling, liquefaction, saccharification, and fermentation. A glucoamylase and an alpha-amylase described herein may be used in saccharification.

The grain is milled in order to open up the structure and allow for further processing. The two processes generally used are wet or dry milling. In dry milling the whole kernel is milled and used in the remaining part of the process. Wet milling gives a very good separation of germ and meal (starch granules and protein) and is, with a few exceptions, applied at locations where there is a parallel production of syrups.

In the liquefaction process, the starch granules are solubilized by hydrolysis to maltodextrins mostly of a DP higher than 4. The hydrolysis may be carried out by acid treatment or enzymatically by alpha-amylase. Acid hydrolysis is used on a limited basis. The raw material can be milled whole grain or a side stream from starch processing. Enzymatic liquefaction is typically carried out as a three-step hot slurry process. The slurry is heated to between about 60-95° C., typically about 80-85° C., and the enzyme(s) is (are) added. Then the slurry is jet-cooked at between about 95-140° C., typically about 105-125° C., cooled to about 60-95° C. and more enzyme(s) is (are) added to obtain the final hydrolysis. The liquefaction process can be carried out at about pH 4.5-6.5, typically at a pH about between about 5.0 and about 6.0. Milled and liquefied grain is also known as mash.

To produce low molecular sugars DP₁₋₃ that can be metabolized by yeast, the maltodextrin from the liquefaction must be further hydrolyzed or saccharified. The hydrolysis is typically performed enzymatically using glucoamylases, alternatively alpha-glucosidases, or acid alpha-amylases. In one embodiment, a glucoamylase and an AmyE or variant thereof are used in saccharification. A full saccharification step may last up to 72 hours, however, it is common only to do a pre-saccharification of typically 40-90 minutes and then complete saccharification during fermentation (SSF). Saccharification is generally carried out at temperatures from about 30-65° C., typically around about 60° C., and at about pH 4.5.

Yeast typically from Saccharomyces spp. is added to the mash and the fermentation is ongoing for 24-96 hours, such as typically 35-60 hours. The temperature is between about 26-34° C., typically at about 32° C., and the pH is from about pH 3-6, typically around about pH 4-5. Note that the most widely used process is a simultaneous saccharification and fermentation (SSF) process where there is no holding stage for the saccharification, meaning that yeast and enzyme is added together. When doing SSF, it is common to introduce a pre-saccharification step at a temperature above 50° C., just prior to the fermentation.

Following the fermentation the mash is distilled to extract the ethanol. The ethanol obtained according to the process of the disclosure may be used as, e.g., fuel ethanol; drinking ethanol, i.e., potable neutral spirits or industrial ethanol. Left over from the fermentation is the grain, which is typically used for animal feed either in liquid form or dried. Further details on how to carry out liquefaction, saccharification, fermentation, distillation, and recovery of ethanol are well known to the skilled person. According to the process of the disclosure, the saccharification and fermentation may be carried out simultaneously or separately.

Although the present compositions, compounds, and methods have been described in detail with reference to examples below, it is understood that various modifications can be made without departing from the described compositions, compounds and methods, and would be readily known to the skilled artisan.

### 5.4. Cleaning and Dishwashing Compositions and Use

The AmyE or variants thereof discussed herein can be formulated in detergent compositions for use in cleaning dishes or other cleaning compositions, for example. These can be gels, powders, or liquids. The compositions can comprise the alpha-amylase variant alone, other amylolytic enzymes, other cleaning enzymes, and other components common to cleaning compositions.

Thus, a dishwashing detergent composition can comprise a surfactant. The surfactant may be anionic, non-ionic, cationic, amphoteric, or a mixture of these types. The detergent can contain about 0% to about 90% by weight of a non-ionic surfactant, such as low- to non-foaming ethoxylated propoxylated straight-chain alcohols.

In the detergent applications, AmyE or variants thereof are usually used in a liquid composition containing propylene glycol. The AmyE or variants thereof can be solubilized in propylene glycol, for example, by circulating in an about 25% volume/volume propylene glycol solution containing about 10% calcium chloride.

The dishwashing detergent composition may contain detergent builder salts of inorganic and/or organic types. The detergent builders may be subdivided into phosphorus-containing and non-phosphorus-containing types. The detergent composition usually contains about 1% to about 90% of detergent builders. Examples of phosphorus-containing inorganic alkaline detergent builders, when present, include the water-soluble salts, especially alkali metal pyrophosphates, orthophosphates, and polyphosphates. An example of phosphorus-containing organic alkaline detergent builder, when present, includes the water-soluble salts of phosphonates. Examples of non-phosphorus-containing inorganic builders, when present, include water-soluble alkali metal carbonates, borates, and silicates, as well as the various types of water-insoluble crystalline or amorphous alumino silicates, of which zeolites are the best-known representatives.

Examples of suitable organic builders include the alkali metal; ammonium and substituted ammonium; citrates; succinates; malonates; fatty acid sulphonates; carboxymethoxy succinates; ammonium polyacetates; carboxylates; polycarboxylates; aminopolycarboxylates; polyacetyl carboxylates; and polyhydroxsulphonates.

Other suitable organic builders include the higher molecular weight polymers and copolymers known to have builder properties, for example appropriate polyacrylic acid, polymaleic and polyacrylic/polymaleic acid copolymers, and their salts.

The cleaning composition may contain bleaching agents of the chlorine/bromine-type or the oxygen-type. Examples of inorganic chlorine/bromine-type bleaches are lithium, sodium or calcium hypochlorite, and hypobromite, as well as chlorinated trisodium phosphate. Examples of organic chlorine/bromine-type bleaches are heterocyclic N-bromo- and N-chloro-imides such as trichloroisocyanuric, tribromoisocyanuric, dibromoisocyanuric, and dichloroisocyanuric acids, and salts thereof with water-solubilizing cations such as potassium and sodium. Hydantoin compounds are also suitable.

The cleaning composition may contain oxygen bleaches, for example in the form of an inorganic persalt, optionally with a bleach precursor or as a peroxy acid compound. Typical examples of suitable peroxy bleach compounds are alkali metal perborates, both tetrahydrates and monohydrates, alkali metal percarbonates, persilicates, and perphosphates. Suitable activator materials include tetraacetylethylenediamine (TAED) and glycerol triacetate. Enzymatic bleach activation systems may also be present, such as perborate or percarbonate, glycerol triacetate and perhydrolase, as disclosed in WO 2005/056783, for example.

The cleaning composition may be stabilized using conventional stabilizing agents for the enzyme(s), e.g., a polyol such as, e.g., propylene glycol, a sugar or a sugar alcohol, lactic acid, boric acid, or a boric acid derivative (e.g., an aromatic borate ester). The cleaning composition may also contain other conventional detergent ingredients, e.g., deflocculant material, filler material, foam depressors, anti-corrosion agents, soil-suspending agents, sequestering agents, anti-soil redeposition agents, dehydrating agents, dyes, bactericides, fluorescent agents, thickeners, and perfumes.

Finally, the AmyE or variants thereof may be used in conventional dishwashing detergents, e.g., in any of the detergents described in the following patent publications, with the consideration that the AmyE or variants thereof disclosed herein are used instead of, or in addition to, any alpha-amylase disclosed in the listed patents and published applications: CA 2006687, GB 2200132, GB 2234980, GB 2228945, DE 3741617, DE 3727911, DE 4212166, DE 4137470, DE 3833047, DE 4205071, WO 93/25651, WO 93/18129, WO 93/04153, WO 92/06157, WO 92/08777, WO 93/21299, WO 93/17089, WO 93/03129, EP 481547, EP 530870, EP 533239, EP 554943, EP 429124, EP 346137, EP 561452, EP 318204, EP 318279, EP 271155, EP 271156, EP 346136, EP 518719, EP 518720, EP 518721, EP 516553, EP 561446, EP 516554, EP 516555, EP 530635, EP 414197, and U.S. Pat. Nos. 5,112,518; 5,141,664; and 5,240,632.

### 5.5. Laundry Detergent Compositions and Use

According to the embodiment, one or more AmyE or variant thereof may be a component of a detergent composition. As such, it may be included in the detergent composition in the form of a non-dusting granulate, a stabilized liquid, or a protected enzyme. Non-dusting granulates may be produced, e.g., as disclosed in U.S. Pat. Nos. 4,106,991 and 4,661,452 and may optionally be coated by methods known in the art. Exemplary waxy coating materials are poly(ethylene oxide) products; (polyethyleneglycol, PEG) with mean molar weights of 1,000 to 20,000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in, for example, GB Patent No. 1,483,591. Liquid enzyme preparations may, for instance, be stabilized by adding a polyol such as propylene glycol, a sugar or sugar alcohol, lactic acid or boric acid according to established methods. Other enzyme stabilizers are well known in the art. Protected enzymes may be prepared according to the method disclosed in U.S. Pat. No. 5,879,920 (Danisco A/S) or EP 238216, for example. Polyols have long been recognized as stabilizers of proteins as well as for improving the solubility of proteins. See, e.g., Kaushik et al., J. Biol. Chem. 278: 26458-65 (2003) and references cited therein; and M. Conti et al., J. Chromatography 757: 237-245 (1997).

The detergent composition may be in any convenient form, e.g., as gels, powders, granules, pastes, or liquids. A liquid detergent may be aqueous, typically containing up to about 70% of water, and 0% to about 30% of organic solvent, it may also be in the form of a compact gel type containing only about 30% water.

The detergent composition comprises one or more surfactants, each of which may be anionic, nonionic, cationic, or zwitterionic. The detergent will usually contain 0% to about 50% of anionic surfactant, such as linear alkylbenzenesulfonate; α-olefinsulfonate; alkyl sulfate (fatty alcohol sulfate) (AS); alcohol ethoxysulfate (AEOS or AES); secondary alkanesulfonates (SAS); α-sulfo fatty acid methyl esters; alkyl- or alkenylsuccinic acid; or soap. The composition may also contain 0% to about 40% of nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide, as described in WO 92/06154, for example.

The detergent composition may additionally comprise one or more other enzymes, such as lipase, cutinase, protease, cellulase, peroxidase, and/or laccase in any combination.

The detergent may contain about 1% to about 65% of a detergent builder or complexing agent such as zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst). The detergent may also be unbuilt, i.e., essentially free of detergent builder. Enzymes may be used in any composition compatible with the stability of the enzyme. Enzymes can be protected against generally deleterious components by known forms of encapsulation, as by granulation or sequestration in hydro gels, for example. Enzymes and specifically alpha-amylases either with or without the starch binding domains are not limited to laundry and dishwashing applications, but may bind use in surface cleaners and ethanol production from starch or biomass.

The detergent may comprise one or more polymers. Examples include carboxymethylcellulose (CMC), poly(vinylpyrrolidone) (PVP), polyethyleneglycol (PEG), poly(vinyl alcohol) (PVA), polycarboxylates such as polyacrylates, maleic/acrylic acid copolymers and lauryl methacrylate/acrylic acid copolymers.

The detergent may contain a bleaching system, which may comprise a H₂O₂ source such as perborate or percarbonate optionally combined with a peracid-forming bleach activator, such as TAED or nonanoyloxybenzenesulfonate (NOBS). Alternatively, the bleaching system may comprise peroxy acids of the amide, imide, or sulfone type, for example. The bleaching system can also be an enzymatic bleaching system where a perhydrolase activates peroxide, such as that described in WO 2005/056783.

The enzymes of the detergent composition may be stabilized using conventional stabilizing agents, e.g., a polyol such as propylene glycol or glycerol; a sugar or sugar alcohol; lactic acid; boric acid or a boric acid derivative, such as an aromatic borate ester; and the composition may be formulated as described in WO 92/19709 and WO 92/19708, for example.

The detergent may also contain other conventional detergent ingredients such as fabric conditioners including clays, foam boosters, suds suppressors, anti-corrosion agents, soil-suspending agents, anti-soil redeposition agents, dyes, bactericides, optical brighteners, or perfume, for example. The pH (measured in aqueous solution at use concentration) is usually neutral or alkaline, e.g., pH about 7.0 to about 11.0.

The alpha-amylase variant may be incorporated in concentrations conventionally employed in detergents. It is at present contemplated that, in the detergent composition, the alpha-amylase variant may be added in an amount corresponding to 0.00001-1.0 mg (calculated as pure enzyme protein) of alpha-amylase variant per liter of wash liquor. Particular forms of detergent compositions comprising the alpha-amylase variants can be formulated to include:

(1) A detergent composition formulated as a granulate having a bulk density of at least 600 g/L comprising linear alkylbenzenesulfonate (calculated as acid) about 7% to about 12%; alcohol ethoxysulfate (e.g., C₁₂₋₁₈ alcohol, 1-2 ethylene oxide (EO)) or alkyl sulfate (e.g., C₁₆₋₁₈) about 1% to about 4%; alcohol ethoxylate (e.g., C₁₄₋₁₅ alcohol, 7 EO) about 5% to about 9%; sodium carbonate (e.g., Na₂CO₃) about 14% to about 20%; soluble silicate, about 2 to about 6%; zeolite (e.g., NaAlSiO₄) about 15% to about 22%; sodium sulfate (e.g., Na₂SO₄) 0% to about 6%; sodium citrate/citric acid (e.g., C₆H₅Na₃O₇/C₆H₈O₇) about 0% to about 15%; sodium perborate (e.g., NaBO₃.H₂O) about 11% to about 18%; TAED about 2% to about 6%; carboxymethylcellulose (CMC) and 0% to about 2%; polymers (e.g., maleic/acrylic acid, copolymer, PVP, PEG) 0-3%; enzymes (calculated as pure enzyme) 0.0001-0.1% protein; and minor ingredients (e.g., suds suppressors, perfumes, optical brightener, photobleach) 0-5%.

(2) A detergent composition formulated as a granulate having a bulk density of at least 600 g/L comprising linear alkylbenzenesulfonate (calculated as acid) about 6% to about 11%; alcohol ethoxysulfate (e.g., C₁₂₋₁₈ alcohol, 1-2 EO) or alkyl sulfate (e.g., C₁₆₋₁₈) about 1% to about 3%; alcohol ethoxylate (e.g., C₁₄₋₁₅ alcohol, 7 EO) about 5% to about 9%; sodium carbonate (e.g., Na₂CO₃) about 15% to about 21%; soluble silicate, about 1% to about 4%; zeolite (e.g., NaAlSiO₄) about 24% to about 34%; sodium sulfate (e.g., Na₂SO₄) about 4% to about 10%; sodium citrate/citric acid (e.g., C₆H₅Na₃O₇/C₆H₈O₇) 0% to about 15%; carboxymethylcellulose (CMC) 0% to about 2%; polymers (e.g., maleic/acrylic acid copolymer, PVP, PEG) 1-6%; enzymes (calculated as pure enzyme protein) 0.0001-0.1%; minor ingredients (e.g., suds suppressors, perfume) 0-5%.

(3) A detergent composition formulated as a granulate having a bulk density of at least 600 g/L comprising linear alkylbenzenesulfonate (calculated as acid) about 5% to about 9%; alcohol ethoxylate (e.g., C₁₂₋₁₅ alcohol, 7 EO) about 7% to about 14%; Soap as fatty acid (e.g., C₁₆₋₂₂ fatty acid) about 1 to about 3%; sodium carbonate (as Na₂CO₃) about 10% to about 17%; soluble silicate, about 3% to about 9%; zeolite (as NaAlSiO₄) about 23% to about 33%; sodium sulfate (e.g., Na₂SO₄) 0% to about 4%; sodium perborate (e.g., NaBO₃.H₂O) about 8% to about 16%; TAED about 2% to about 8%; phosphonate (e.g., EDTMPA) 0% to about 1%; carboxymethylcellulose (CMC) 0% to about 2%; polymers (e.g., maleic/acrylic acid copolymer, PVP, PEG) 0-3%; enzymes (calculated as pure enzyme protein) 0.0001-0.1%; minor ingredients (e.g., suds suppressors, perfume, optical brightener) 0-5%.

(4) A detergent composition formulated as a granulate having a bulk density of at least 600 g/L comprising linear alkylbenzenesulfonate (calculated as acid) about 8% to about 12%; alcohol ethoxylate (e.g., C₁₂₋₁₅ alcohol, 7 EO) about 10% to about 25%; sodium carbonate (as Na₂CO₃) about 14% to about 22%; soluble silicate, about 1% to about 5%; zeolite (e.g., NaAlSiO₄) about 25% to about 35%; sodium sulfate (e.g., Na₂SO₄) 0% to about 10%; carboxymethylcellulose (CMC) 0% to about 2%; polymers (e.g., maleic/acrylic acid copolymer, PVP, PEG) 1-3%; enzymes (calculated as pure enzyme protein) 0.0001-0.1%; and minor ingredients (e.g., suds suppressors, perfume) 0-5%.

(5) An aqueous liquid detergent composition comprising linear alkylbenzenesulfonate (calculated as acid) about 15% to about 21%; alcohol ethoxylate (e.g., C₁₂₋₁₅ alcohol, 7 EO or C₁₂₋₁₅ alcohol, 5 EO) about 12% to about 18%; soap as fatty acid (e.g., oleic acid) about 3% to about 13%; alkenylsuccinic acid (C₁₂₋₁₄) 0% to about 13%; aminoethanol about 8% to about 18%; citric acid about 2% to about 8%; phosphonate 0% to about 3%; polymers (e.g., PVP, PEG) 0% to about 3%; borate (e.g., B₄O₇) 0% to about 2%; ethanol 0% to about 3%; propylene glycol about 8% to about 14%; enzymes (calculated as pure enzyme protein) 0.0001-0.1%; and minor ingredients (e.g., dispersants, suds suppressors, perfume, optical brightener) 0-5%.

(6) An aqueous structured liquid detergent composition comprising linear alkylbenzenesulfonate (calculated as acid) about 15% to about 21%; alcohol ethoxylate (e.g., C₁₂₋₁₅ alcohol, 7 EO, or C₁₂₋₁₅ alcohol, 5 EO) 3-9%; soap as fatty acid (e.g., oleic acid) about 3% to about 10%; zeolite (as NaAlSiO₄) about 14% to about 22%; potassium citrate about 9% to about 18%; borate (e.g., B₄O₇) 0% to about 2%; carboxymethylcellulose (CMC) 0% to about 2%; polymers (e.g., PEG, PVP) 0% to about 3%; anchoring polymers (e.g., lauryl methacrylate/acrylic acid copolymer); molar ratio 25:1, MW 3800) 0% to about 3%; glycerol 0% to about 5%; enzymes (calculated as pure enzyme protein) 0.0001-0.1%; and minor ingredients (e.g., dispersants, suds suppressors, perfume, optical brighteners) 0-5%.

(7) A detergent composition formulated as a granulate having a bulk density of at least 600 g/L comprising fatty alcohol sulfate about 5% to about 10%; ethoxylated fatty acid monoethanolamide about 3% to about 9%; soap as fatty acid 0-3%; sodium carbonate (e.g., Na₂CO₃) about 5% to about 10%; soluble silicate, about 1% to about 4%; zeolite (e.g., NaAlSiO₄) about 20% to about 40%; sodium sulfate (e.g., Na₂SO₄) about 2% to about 8%; sodium perborate (e.g., NaBO₃.H₂O) about 12% to about 18%; TAED about 2% to about 7%; polymers (e.g., maleic/acrylic acid copolymer, PEG) about 1% to about 5%; enzymes (calculated as pure enzyme protein) 0.0001-0.1%; and minor ingredients (e.g., optical brightener, suds suppressors, perfume) 0-5%.

(8) A detergent composition formulated as a granulate comprising linear alkylbenzenesulfonate (calculated as acid) about 8% to about 14%; ethoxylated fatty acid monoethanolamide about 5% to about 11%; soap as fatty acid 0% to about 3%; sodium carbonate (e.g., Na₂CO₃) about 4% to about 10%; soluble silicate, about 1% to about 4%; zeolite (e.g., NaAlSiO₄) about 30% to about 50%; sodium sulfate (e.g., Na₂SO₄) about 3% to about 11%; sodium citrate (e.g., C₆H₅Na₃O₇) about 5% to about 12%; polymers (e.g., PVP, maleic/acrylic acid copolymer, PEG) about 1% to about 5%; enzymes (calculated as pure enzyme protein) 0.0001-0.1%; and minor ingredients (e.g., suds suppressors, perfume) 0-5%.

(9) A detergent composition formulated as a granulate comprising linear alkylbenzenesulfonate (calculated as acid) about 6% to about 12%; nonionic surfactant about 1% to about 4%; soap as fatty acid about 2% to about 6%; sodium carbonate (e.g., Na₂CO₃) about 14% to about 22%; zeolite (e.g., NaAlSiO₄) about 18% to about 32%; sodium sulfate (e.g., Na₂SO₄) about 5% to about 20%; sodium citrate (e.g., C₆H₅Na₃O₇) about 3% to about 8%; sodium perborate (e.g., NaBO₃.H₂O) about 4% to about 9%; bleach activator (e.g., NOBS or TAED) about 1% to about 5%; carboxymethylcellulose (CMC) 0% to about 2%; polymers (e.g., polycarboxylate or PEG) about 1% to about 5%; enzymes (calculated as pure enzyme protein) 0.0001-0.1%; and minor ingredients (e.g., optical brightener, perfume) 0-5%.

(10) An aqueous liquid detergent composition comprising linear alkylbenzenesulfonate (calculated as acid) about 15% to about 23%; alcohol ethoxysulfate (e.g., C₁₂₋₁₅ alcohol, 2-3 EO) about 8% to about 15%; alcohol ethoxylate (e.g., C₁₂₋₁₅ alcohol, 7 EO, or C₁₂₋₁₅ alcohol, 5 EO) about 3% to about 9%; soap as fatty acid (e.g., lauric acid) 0% to about 3%; aminoethanol about 1% to about 5%; sodium citrate about 5% to about 10%; hydrotrope (e.g., sodium toluensulfonate) about 2% to about 6%; borate (e.g., B₄O₇) 0% to about 2%; carboxymethylcellulose 0% to about 1%; ethanol about 1% to about 3%; propylene glycol about 2% to about 5%; enzymes (calculated as pure enzyme protein) 0.0001-0.1%; and minor ingredients (e.g., polymers, dispersants, perfume, optical brighteners) 0-5%.

(11) An aqueous liquid detergent composition comprising linear alkylbenzenesulfonate (calculated as acid) about 20% to about 32%; alcohol ethoxylate (e.g., C₁₂₋₁₅ alcohol, 7 EO, or C₁₂₋₁₅ alcohol, 5 EO) 6-12%; aminoethanol about 2% to about 6%; citric acid about 8% to about 14%; borate (e.g., B₄O₇) about 1% to about 3%; polymer (e.g., maleic/acrylic acid copolymer, anchoring polymer, such as lauryl methacrylate/acrylic acid copolymer) 0% to about 3%; glycerol about 3% to about 8%; enzymes (calculated as pure enzyme protein) 0.0001-0.1%; and minor ingredients (e.g., hydrotropes, dispersants, perfume, optical brighteners) 0-5%.

(12) A detergent composition formulated as a granulate having a bulk density of at least 600 g/L comprising anionic surfactant (linear alkylbenzenesulfonate, alkyl sulfate, α-olefinsulfonate, α-sulfo fatty acid methyl esters, alkanesulfonates, soap) about 25% to about 40%; nonionic surfactant (e.g., alcohol ethoxylate) about 1% to about 10%; sodium carbonate (e.g., Na₂CO₃) about 8% to about 25%; soluble silicates, about 5% to about 15%; sodium sulfate (e.g., Na₂SO₄) 0% to about 5%; zeolite (NaAlSiO₄) about 15% to about 28%; sodium perborate (e.g., NaBO₃H₂O) 0% to about 20%; bleach activator (TAED or NOBS) about 0% to about 5%; enzymes (calculated as pure enzyme protein) 0.0001-0.1%; minor ingredients (e.g., perfume, optical brighteners) 0-3%.

(13) Detergent compositions as described in compositions 1)-12) supra, wherein all or part of the linear alkylbenzenesulfonate is replaced by (C₁₂-C₁₈) alkyl sulfate.

(14) A detergent composition formulated as a granulate having a bulk density of at least 600 g/L comprising (C₁₂-C₁₈) alkyl sulfate about 9% to about 15%; alcohol ethoxylate about 3% to about 6%; polyhydroxy alkyl fatty acid amide about 1% to about 5%; zeolite (e.g., NaAlSiO₄) about 10% to about 20%; layered disilicate (e.g., SK56 from Hoechst) about 10% to about 20%; sodium carbonate (e.g., Na₂CO₃) about 3% to about 12%; soluble silicate, 0% to about 6%; sodium citrate about 4% to about 8%; sodium percarbonate about 13% to about 22%; TAED about 3% to about 8%; polymers (e.g., polycarboxylates and PVP) 0% to about 5%; enzymes (calculated as pure enzyme protein) 0.0001-0.1%; and minor ingredients (e.g., optical brightener, photobleach, perfume, suds suppressors) 0-5%.

(15) A detergent composition formulated as a granulate having a bulk density of at least 600 g/L comprising (C₁₂-C₁₈) alkyl sulfate about 4% to about 8%; alcohol ethoxylate about 11% to about 15%; soap about 1% to about 4%; zeolite MAP or zeolite A about 35% to about 45%; sodium carbonate (as Na₂CO₃) about 2% to about 8%; soluble silicate, 0% to about 4%; sodium percarbonate about 13% to about 22%; TAED 1-8%; carboxymethylcellulose (CMC) 0% to about 3%; polymers (e.g., polycarboxylates and PVP) 0% to about 3%; enzymes (calculated as pure enzyme protein) 0.0001-0.1%; and minor ingredients (e.g., optical brightener, phosphonate, perfume) 0-3%.

(16) Detergent formulations as described in 1)-15) supra, which contain a stabilized or encapsulated peracid, either as an additional component or as a substitute for already specified bleach systems.

(17) Detergent compositions as described supra in 1), 3), 7), 9), and 12), wherein perborate is replaced by percarbonate.

(18) Detergent compositions as described supra in 1), 3), 7), 9), 12), 14), and 15), which additionally contains a manganese catalyst.

(19) Detergent composition formulated as a non-aqueous detergent liquid comprising a liquid nonionic surfactant such as, e.g., linear alkoxylated primary alcohol, a builder system (e.g., phosphate), an enzyme(s), and alkali. The detergent may also comprise anionic surfactant and/or a bleach system.

In another embodiment, the 2,6-β-D-fructan hydrolase can be incorporated in detergent compositions and used for removal/cleaning of biofilm present on household and/or industrial textile/laundry.

The detergent composition may for example be formulated as a hand or machine laundry detergent composition, including a laundry additive composition suitable for pre-treatment of stained fabrics and a rinse added fabric softener composition, or can be formulated as a detergent composition for use in general household hard surface cleaning operations, or be formulated for hand or machine dishwashing operations.

In a specific aspect, the detergent composition can comprise 2,6-β-D-fructan hydrolase, one or more alpha-amylase variants, and one or more other cleaning enzymes, such as a protease, a lipase, a cutinase, a carbohydrase, a cellulase, a pectinase, a mannanase, an arabinase, a galactanase, a xylanase, an oxidase, a laccase, and/or a peroxidase, and/or combinations thereof. In general the properties of the chosen enzyme(s) should be compatible with the selected detergent, (e.g., pH-optimum, compatibility with other enzymatic and non-enzymatic ingredients, etc.), and the enzyme(s) should be present in effective amounts.

Proteases: suitable proteases include those of animal, vegetable or microbial origin. Chemically modified or protein engineered mutants are also suitable. The protease may be a serine protease or a metalloprotease, e.g., an alkaline microbial protease or a trypsin-like protease. Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115. Suitable commercially available protease enzymes include Alcalase®, Savinase®, Primase™, Duralase™, Esperase®, and Kannase™ (Novo Nordisk A/S); Maxatase®, Maxacal™, Maxapem™, Properase™, Purafect®, Purafect OxP™, FN2™, and FN3™ (Danisco A/S).

Lipases: suitable lipases include those of bacterial or fungal origin. Chemically modified or protein engineered mutants are included. Examples of useful lipases include, but are not limited to, lipases from Humicola (synonym Thermomyces), e.g. H. lanuginosa (T. lanuginosus) (see, e.g., EP 258068 and EP 305216) and H. insolens (see, e.g., WO 96/13580); a Pseudomonas lipase (e.g., from P. alcaligenes or P. pseudoalcaligenes; see, e.g., EP 218 272), P. cepacia (see, e.g., EP 331 376), P. stutzeri (see, e.g., GB 1,372,034), P. fluorescens, Pseudomonas sp. strain SD 705 (see, e.g., WO 95/06720 and WO 96/27002), P. wisconsinensis (see, e.g., WO 96/12012); a Bacillus lipase (e.g., from B. subtilis; see, e.g., Dartois et al. Biochemica Biophysica Acta, 1131: 253-360 (1993)), B. stearothermophilus (see, e.g., JP 64/744992), or B. pumilus (see, e.g., WO 91/16422). Additional lipase variants contemplated for use in the formulations include those described, for example, in: WO 92/05249, WO 94/01541, WO 95/35381, WO 96/00292, WO 95/30744, WO 94/25578, WO 95/14783, WO 95/22615, WO 97/04079, WO 97/07202, EP 407225, and EP 260105. Some commercially available lipase enzymes include Lipolase® and Lipolase® Ultra (Novo Nordisk A/S).

Polyesterases: Suitable polyesterases include, but are not limited to, those described in WO 01/34899 (Danisco A/S) and WO 01/14629 (Danisco A/S), and can be included in any combination with other enzymes discussed herein.

Amylases: The compositions can be combined with other alpha-amylases, such as a non-variant alpha-amylase. These can include commercially available amylases, such as but not limited to Duramyl®, Termamyl™, Fungamyl® and BAN™ (Novo Nordisk A/S), Rapidase®, and Purastar® (Danisco A/S).

Cellulases: Cellulases can be added to the compositions. Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307; 5,648,263; 5,691,178; 5,776,757; and WO 89/09259, for example. Exemplary cellulases contemplated for use are those having color care benefit for the textile. Examples of such cellulases are cellulases described in EP 0495257; EP 531 372; WO 99/25846 (Danisco A/S), WO 96/34108 (Danisco A/S), WO 96/11262; WO 96/29397; and WO 98/08940, for example. Other exemplary cellulase variants, include those described in WO 94/07998; WO 98/12307; WO 95/24471; PCT/DK98/00299; EP 531 315; U.S. Pat. Nos. 5,457,046; 5,686,593; and 5,763,254. Commercially available cellulases include Celluzyme® and Carezyme® (Novo Nordisk A/S); Clazinase™ and Puradax® HA (Danisco A/S); and KAC-500(B)™ (Kao Corporation).

Peroxidases/Oxidases: Suitable peroxidases/oxidases contemplated for use in the compositions include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novo Nordisk A/S), for example.

The detergent enzyme(s) may be included in a detergent composition by adding separate additives containing one or more enzymes, or by adding a combined additive comprising all of these enzymes. A detergent additive, i.e., a separate additive or a combined additive, can be formulated as a granulate, liquid, slurry, etc. Suitable granulate detergent additive formulations include non-dusting granulates.

Non-dusting granulates may be produced, e.g., as disclosed in U.S. Pat. Nos. 4,106,991 and 4,661,452 and optionally may be coated by methods known in the art. Examples of waxy coating materials are poly(ethylene oxide) products (e.g., polyethyleneglycol, PEG) with mean molar weights of 1,000 to 20,000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591, for example. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238 216.

The detergent composition may be in any convenient form, e.g., a bar, tablet, gel, powder, granule, paste, or liquid. A liquid detergent may be aqueous, typically containing up to about 70% water, and 0% to about 30% organic solvent. Compact detergent gels containing 30% or less water are also contemplated. The detergent composition comprises one or more surfactants, which may be non-ionic, including semi-polar, anionic, cationic, or zwitterionic, or any combination thereof. The surfactants are typically present at a level of from 0.1% to 60% by weight.

When included therein the detergent typically will contain from about 1% to about 40% of an anionic surfactant, such as linear alkylbenzenesulfonate, α-olefinsulfonate, alkyl sulfate (fatty alcohol sulfate), alcohol ethoxysulfate, secondary alkanesulfonate, α-sulfo fatty acid methyl ester, alkyl- or alkenylsuccinic acid, or soap.

When included therein, the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl-N-alkyl derivatives of glucosamine (“glucamides”).

The detergent may contain 0% to about 65% of a detergent builder or complexing agent such as zeolite, diphosphate, triphosphate, phosphonate, carbonate, citrate, nitrilotriacetic acid, ethylenediaminetetraacetic acid (EDTA), diethylenetriaminepentaacetic acid, alkyl- or alkenylsuccinic acid, soluble silicates or layered silicates (e.g., SKS-6 from Hoechst).

The detergent may comprise one or more polymers. Examples are carboxymethylcellulose (CMC), poly(vinylpyrrolidone) (PVP), poly(ethylene glycol) (PEG), poly(vinyl alcohol) (PVA), poly(vinylpyridine-N-oxide), poly(vinylimidazole), polycarboxylates, e.g., polyacrylates, maleic/acrylic acid copolymers), and lauryl methacrylate/acrylic acid copolymers.

The detergent may contain a bleaching system that may comprise a source of H₂O₂, such as perborate or percarbonate, which may be combined with a peracid-forming bleach activator (e.g., tetraacetylethylenediamine or nonanoyloxybenzenesulfonate). Alternatively, the bleaching system may comprise peroxyacids (e.g., the amide-, imide-, or sulfone-type peroxyacids). The bleaching system can also be an enzymatic bleaching system.

The enzyme(s) of the detergent composition may be stabilized using conventional stabilizing agents, e.g., polyol (e.g., propylene glycol or glycerol), a sugar or sugar alcohol, lactic acid, boric acid, a boric acid derivative (e.g., an aromatic borate ester), or a phenyl boronic acid derivative (e.g., 4-formylphenyl boronic acid). The composition may be formulated as described in WO 92/19709 and WO 92/19708.

The detergent may also contain other conventional detergent ingredients such as e.g., fabric conditioners including clays, foam boosters, suds suppressors, anti-corrosion agents, soil-suspending agents, anti-soil redeposition agents, dyes, bactericides, optical brighteners, hydrotropes, tarnish inhibitors, or perfumes.

It is contemplated that in the detergent compositions, the enzyme variants may be added in an amount corresponding to about 0.01 to about 100 mg of enzyme protein per liter of wash liquor, particularly about 0.05 to about 5.0 mg of enzyme protein per liter of wash liquor, or even more particularly in 0.1 to about 1.0 mg of enzyme protein per liter of wash liquor.

A representative assay that may be used to test the efficacy of a cleaning composition comprising AmyE or a variant thereof includes a swatch test. A “swatch” is a piece of material such as a fabric that has a stain applied thereto. The material can be, for example, fabrics made of cotton, polyester or mixtures of natural and synthetic fibers. Alternatively, the material can be paper, such as filter paper or nitrocellulose, or a piece of a hard material, such as ceramic, metal, or glass. For alpha-amylases, the stain is starch based, but can include blood, milk, ink, grass, tea, wine, spinach, gravy, chocolate egg, cheese, clay, pigment, oil, or mixtures of these compounds. In one embodiment, the AmyE or variant thereof is tested in a BMI (blood/milk/ink) assay.

A “smaller swatch” is a piece of the swatch that has been cut with a single hole punch device, or a custom manufactured 96-hole punch device, where the pattern of the multi-hole punch is matched to standard 96-well microtiter plates, or has been otherwise removed from the swatch. The swatch can be of textile, paper, metal, or other suitable material. The smaller swatch can have the stain affixed either before or after it is placed into the well of a 24-, 48- or 96-well microtiter plate. The smaller swatch also can be made by applying a stain to a small piece of material. For example, the smaller swatch can be a piece of fabric with a stain 5/8″ or 0.25″ in diameter. The custom manufactured punch can be designed in such a manner that it delivers 96 swatches simultaneously to all wells of a 96-well plate. The device allows delivery of more than one swatch per well by simply loading the same 96-well plate multiple times. Multi-hole punch devices can be conceived to deliver simultaneously swatches to any format plate, including, but not limited to, 24-well, 48-well, and 96-well plates. In another conceivable method, the soiled test platform can be a bead made of either metal, plastic, glass, ceramic, or other suitable material that is coated with the soil substrate. The one or more coated beads are then placed into wells of 96-, 48-, or 24-well plates or larger formats, containing suitable buffer and enzyme. In this case, supernatant can be examined for released soil either by direct absorbance measurement or after a secondary color development reaction. Analysis of the released soil might also be taken by mass spectral analysis.

In one embodiment, a treatment protocol provides control over degree of fixation of a stain. As a result, it is possible to produce swatches that, for example, release varying amounts of stain when washed in the absence of the enzyme being tested. The use of fixed swatches leads to a dramatic improvement of the signal-to-noise ratio in the wash assays. Furthermore, by varying the degree of fixation, one can generate stains that give optimum results under the various cleaning conditions.

Swatches having stains of known “strength” on various types of material are commercially available (EMPA, St. Gallen, Switzerland; wfk—Testgewebe GmbH, Krefeld Germany; or Center for Test Materials, Vlaardingen, The Netherlands) and/or can be made by the practitioner (Morris and Prato, Textile Research Journal 52(4): 280-286 (1982)). Swatches can comprise, for example, a cotton-containing fabric containing a stain made by blood/milk/ink (BMI), spinach, grass, or chocolate/milk/soot. A BMI stain can be fixed to cotton with 0.0003% to 0.3% hydrogen peroxide, for example. Other combinations include grass or spinach fixed with 0.001% to 1% glutaraldehyde, gelatin and Coomassie stain fixed with 0.001% to 1% glutaraldehyde, or chocolate, milk and soot fixed with 0.001% to 1% glutaraldehyde.

The swatch can also be agitated during incubation with the enzyme and/or detergent formulation. Wash performance data is dependent on the orientation of the swatches in the wells (horizontal versus vertical), particularly in the 96-well plate. This would indicate that mixing was insufficient during the incubation period. Although there are a number of ways to ensure sufficient agitation during incubation, a plate holder in which the microtiter plate is sandwiched between two plates of aluminum can be constructed. This can be as simple as placing, for example, an adhesive plate sealer over the wells then clamping the two aluminum plates to the 96-well plate with any type of appropriate, commercially available clamps. It can then be mounted in a commercial incubator shaker. Setting the shaker to about 400 rpm results in very efficient mixing, while leakage or cross-contamination is efficiently prevented by the holder.

Trinitrobenzenesulfonic acid (TNBS) can be used to quantify the concentration of amino groups in the wash liquor. This can serve as a measure of the amount of protein that was removed from the swatch (see, e.g., Cayot and Tainturier, Anal. Biochem. 249: 184-200 (1997)). However, if a detergent or an enzyme sample leads to the formation of unusually small peptide fragments (for example, from the presence of peptidases in the sample), then one will obtain a larger TNBS signal, i.e., more “noise.”

Another means of measuring wash performance of blood/milk/ink that is based on ink release that can be quantified by measuring the absorbance of the wash liquor. The absorbance can be measured at any wavelength between 350 and 800 nm. In one embodiment, the wavelength is measured at 410 nm or 620 nm. The wash liquor can also be examined to determine the wash performance on stains containing grass, spinach, gelatin or Coomassie stain. Suitable wavelengths for these stains include and 670 nm for spinach or grass and 620 nm for gelatin or Coomassie. For example, an aliquot of the wash liquor (typically 100-150 μL from a 96-well microplate, for example) is removed and placed in a cuvette or multiwell microplate. This is then placed in a spectrophotometer and the absorbance is read at an appropriate wavelength. The system also can be used to determine a suitable enzyme and/or detergent composition for dish washing, for example, using a blood/milk/ink stain on a suitable substrate, such as cloth, plastic or ceramic.

In one aspect, a BMI stain is fixed to cotton by applying 0.3% hydrogen peroxide to the BMI/cotton swatch for 30 minutes at 25° C. or by applying 0.03% hydrogen peroxide to the BMI/cotton swatch for 30 minutes at 60° C. Smaller swatches of approximately 0.25″ are cut from the BMI/cotton swatch and placed in the wells of a 96-well microtiter plate. Into each well, a known mixture of a detergent composition and an enzyme, such as a variant protein, is placed. After placing an adhesive plate sealer onto the top of the microtiter plate, the microtiter plate is clamped to an aluminum plate and agitated on an orbital shaker at approximately 250 rpm for about 10 to 60 minutes. At the end of this time, the supernatants are transferred to wells in a new microtiter plate and the absorbance of the ink at 620 nm is measured. This can be similarly tests with spinach stains or grass stains fixed to cotton by applying 0.01% glutaraldehyde to the spinach/cotton swatch or grass/cotton swatch for 30 minutes at 25° C. The same can be done with chocolate, milk, and/or soot stains.

### 5.6. Textile Desizing Compositions and Use

Also contemplated are compositions and methods of treating fabrics (e.g., to desize a textile) using one or more AmyE or variant thereof. The AmyE or variants thereof can be used in any fabric-treating method, which are well known in the art (see, e.g., U.S. Pat. No. 6,077,316). For example, in one aspect, the feel and appearance of a fabric is improved by a method comprising contacting the fabric with an enzyme variant in a solution. In one aspect, the fabric is treated with the solution under pressure.

In one aspect, the enzymes are applied during or after the weaving of textiles, or during the desizing stage, or one or more additional fabric processing steps. During the weaving of textiles, the threads are exposed to considerable mechanical strain. Prior to weaving on mechanical looms, warp yarns are often coated with sizing starch or starch derivatives in order to increase their tensile strength and to prevent breaking. The AmyE or variants thereof can be applied to remove these sizing starch or starch derivatives. After the textiles have been woven, a fabric can proceed to a desizing stage. This can be followed by one or more additional fabric processing steps. Desizing is the act of removing size from textiles. After weaving, the size coating should be removed before further processing the fabric in order to ensure a homogeneous and wash-proof result. Also provided is a method of desizing comprising enzymatic hydrolysis of the size by the action of an enzyme variant.

The AmyE or variants thereof can be used alone or with other desizing chemical reagents and/or desizing enzymes to desize fabrics, including cotton-containing fabrics, as detergent additives, e.g., in aqueous compositions. The AmyE or variants thereof also can be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of amylolytic enzymes to provide softness to the fabric and make the cotton more accessible to the subsequent enzymatic finishing steps. The alpha-amylase variant can be used in methods of finishing denim garments (e.g., a “bio-stoning process”), enzymatic desizing and providing softness to fabrics, and/or finishing process.

It will be apparent to those skilled in the art that various modifications and variation can be made to the compositions and methods of using same without departing from the spirit or scope of the intended use. Thus, it is the modifications and variations provided they come within the scope of the appended claims and their equivalents.

### EXAMPLES

### Example 1

### Plasmid Construction

Nucleic acids encoding the AmyE of SEQ ID NO: 1 or a C-terminal truncated AmyE variant, AmyE-tr (SEQ ID NO: 3), were cloned into the B. subtilis pHPLT expression vector, which is disclosed in U.S. Pat. No. 5,024,943. FIG. 4 depicts the vector comprising a nucleic acid encoding AmyE-tr.

Referring to FIG. 4, the pHPLT vector contains the B. licheniformis LAT promoter (“Plat”), a sequence encoding the LAT signal peptide (“preLAT”), followed by PstI and HpaI restriction sites for cloning. Additional plasmid elements from plasmid pUB110 disclosed in McKenzie et al., Plasmid 15(2): 93-103 (1986): “ori-pUB” is the origin of replication from pUB110; “reppUB” is the replicase gene from pUB110, “neo” is the neomycin/kanamycin resistance gene from pUB110; “bleo” is the bleomycin resistance marker, “Tlat” is the transcriptional terminator from B. licheniformis amylase.

Plasmid constructs for the expression of AmyE and AmyE-tr were assembled using the AmyE-encoding sequence described by Yang et al, “Nucleotide sequence of the amylase gene from Bacillus subtilis,” Nucleic Acids Res. 11(2): 237-49 (1983). Plasmid pME629.5 contains the nucleic acid encoding the full-length AmyE of SEQ ID NO: 1. The gene has a three base deletion in the sequence encoding the starch binding domain, compared to the sequence described by Yang et al.

Plasmid pME630.7 contains the truncated AmyE sequence, AmyE-tr, and is shown in FIG. 4. AmyE-tr is truncated at D425 of SEQ ID NO: 1. AmyE-tr was designed from a crystal structure of an AmyE variant that lacks the starch binding domain, disclosed in Fujimoto et al., “Crystal structure of a catalytic-site mutant alpha-amylase from Bacillus subtilis complexed with maltopentaose,” J. Mol. Biol. 277: 393-407 (1998). See RCSB Protein Data Bank© Accession No. 1BAG, “Alpha-Amylase From Bacillus Subtilis Complexed With Maltopentaose.”

For expression plasmid construction, the nucleic acid encoding AmyE was PCR-amplified using Herculase® (Stratagene, Calif.). The PCR products were purified using a column provided in a Qiagen QJAquik™ PCR purification kit (Qiagen, Valencia, Calif.), and resuspended in 50 μL of Milli-Q™-purified water. 50 μL of the purified DNA was digested sequentially with HpaI (Roche) and PstI (Roche), and the resultant DNA resuspended in 30 μL of Milli-Q™-purified water. 10-20 ng/μL DNA was cloned into plasmid pHPLT using PstI and HpaI cloning sites. The ligation mixtures were directly transformed into competent B. subtilis cells (genotype: DaprE, DnprE, degUHy32 oppA, DspoIIE3501, amyE::xylRPxylAcomK-phleo). SC6.1 B. subtilis cells have a competency gene (comK) that is placed under a xylose-inducible promoter. Competency for DNA binding and uptake is induced by the addition of xylose. Because the AmyE gene in the parent plasmid has two PstI sites, a PCR fusion reaction was carried out to remove these sites before cloning. PCR fusion was done after two separate PCR reactions. The following primers were used for making the pHPLT construct using HpaI and PstI sites:

SEQ ID NO: 18: Primer PSTAMYE-F 5′
CTTCTTGCTGCCTCATTCTGCAGCTTCAGCACTTACAGCACCGTCGATC
AAAAGCGGAAC 3′
SEQ ID NO: 19: Primer AMYENOPST-R 5′
CTGGAGGCACTATCCTGAAGGATTTCTCCGTATTGGAACTCTGCTGATG
TATTTGTG 3′
SEQ ID NO: 20: Primer AMYENOPST-F 5′
CACAAATACATCAGCAGAGTTCCAATACGGAGAAATCCTTCAGGATAGT
GCCTCCAG 3′
SEQ ID NO: 21: Primer HPAIAMYE-R 5′
CAGGAAATCCGTCCTCTGTTAACTCAATGGGGAAGAGAACCGCTTAAGC
CCGAGTC 3′
SEQ ID NO: 22: Primer HPAIAMYE466-R 5′
CAGGAAATCCGTCCTCTGTTAACTCAATCAGGATAAAGCACAGCTACAG
ACCTGG 3′
SEQ ID NO: 23: Primer AMYE SEQ-F1
5′ TACACAAGTACAGTCCTATCTG 3′
SEQ ID NO: 24: Primer AMYE SEQ-F2
5′ CATCCTCTGTCTCTATCAATAC 3′

The plasmids pME629.5 and pME630.7 express AmyE with a 31 residue signal sequence, which is cleaved post-translationally. The subsequent 10 N-terminal amino acids are processed separately as proposed by Yang et al. (1983) supra.

Protein Expression

Transformants for AmyE full-length and truncated clones were selected on LA with 10 μg/ml neomycin, 1% insoluble starch, and incubated overnight at 37° C. Transformants showing a clearing (or halo) around the colony were selected, and vials were made for further studies. Pre-cultures of the transformants were grown for 8 hr in LB with 10 μg/mL neomycin. Then, 30 μL of this pre-culture were added into a 250 mL flask filled with 30 mL of cultivation media (described below) supplemented with 10 μg/mL neomycin and 5 mM CaCl₂. The cultivation media was an enriched semi-defined media based on MOPS buffer, with urea as the major nitrogen source, glucose generally as the main carbon source, and supplemented with 1% soytone for robust cell growth. The shake flasks were incubated for 60-65 hours at about 37° C., with mixing at 250 rpm. Cultures were harvested by centrifugation at 5000 rpm for 20 minutes in conical tubes. Since both AmyE full-length and AmyE truncated proteins expressed at high levels, the culture supernatants were used for assays without further purification.

### Example 2

The following assays were used in the examples described below. Any deviations from the protocols provided below are indicated in the examples. In these experiments, a spectrophotometer was used to measure the absorbance of the products formed after the completion of the reactions.

Bradford Assay for Protein Content Determination in 96-Well Microtiter Plate

Protein concentration in sample supernatants was determined using the Bradford QuickStart™ Dye Reagent (Bio-Rad, Calif.). Samples were obtained by filtration of broths from cultures grown in microtiter plates (MTPs) for 3 days at about 37° C. with shaking at 280 rpm and humidified aeration. A 10 μL sample of the culture filtrate was combined with 200 μL Bradford QuickStart™ Dye Reagent in a well of a second MTP. After thorough mixing, the MTP's were incubated for at least 10 minutes at room temperature. Air bubbles were removed and the OD (optical density) was measured at 595 nm. To determine the protein concentration, the background reading (from uninoculated wells) was subtracted from the sample readings.

Jet Operation Method

The jet operation method described herein is for the use of a HYDRYTERMAL jet skid (also known as the ATTEC cooking system) equipped with an M-101 HydroHeater containing a 0.09″ diameter combining tube (Hydrothermal, Waukesha, Wis.). The system consisted of a supply tank with a stirrer, a positive displacement pump (Moyno) (Moyno Inc., Springfield Ohio), the M-101 HydroHeater brand steam injection cooker, a steam supply, temperature sensors, pressure indicators, hold loops of various lengths, a back pressure valve at the exit (allow cooking at above ambient pressures), and a flash tank.

The system can be used to simulate the operation of steam injection systems as found in large-scale production plants, generally known as the first stage or primary liquefaction. The typical process variables include enzyme dose, jet temperature, primary hold time, pH, calcium and sodium levels, starch quality from the mill house, and dry substance. In general, the quality of the liquefact produced cannot be determined by DE development alone and must be evaluated following saccharification using tests for sediment, filtration, and starch positive (iodine) detection as described below. The starch was made up in an auxiliary tank to the approximate ds target, and then transferred to the tank on the skid by filtering through a 100 mesh screen to exclude any particulate materials that are large enough to plug the small combining tube.

The jet operation consists of three main stages: jet start-up, starch cooking, and shut-down. The following steps were performed at the jet start-up stage:
start water through the system by providing enough flow to the top of the Moyno inlet to overflow to the drain;
start the Moyno at 0.5 gpm (if there is enough air in the system, the flow meter may not detect flow, the system may determined it is dry, and the pump may stop; to solve this problem, air is cleared from the system by pressing the pump stop button and restart);
fill the system with water and set the back pressure valve to about 16-20 psi;
inspect the joints for leaks;
after the system is full of water, open the main steam valve and allow at least a couple of minutes for the line condensate to exit the traps;
open the air supply valve;
turn the temperature selector switch to on, so that the steam is added to the system;
adjust the steam supply to the jet using the micrometer to achieve a temperature reading of about 108.9-110° C. at the resistance temperature detector (RTD);
adjust the combining tube with water at a flow of 0.5 gpm to give a feed pressure of about 40 psi; and
allow the system to operate about one hour, pre-heating all the pipes, joints, pipe anchors, etc.

At the end of the start-up stage, the system should be thoroughly heated, and the thermocouples should read out with less than 1.7° C. drop across the system.

Next, starch slurry was added and cooked. It has been previously determined that 35-40 kg of slurry is the smallest quantity that will provide a suitable sample. This also depends on the requested hold time. With an approximate 6 min hold time, 35-40 kg of slurry (one 3-gallon loop; 29-34 liters or 7.7-9 gallons) will provide 15-18 min of flow. It is believed that the starch slurry will force the water from the system due to the density difference, while chasing water after starch will result in channeling through the system due to density. Accordingly, the starch slurry is used at a minimum amount with the assumption that the system will come to equilibrium rapidly. When working at non-optimum conditions, more slurry may be used to allow more time to reach stability.

Starch slurry was first added to the feed tank, where the pH was adjusted and all reagents (e.g., enzymes) were added. Temperature was reduced to about 98.9° C. at the RTD. Because the starch slurry contains less water per volume and requires less heat from the steam to achieve the target temperature, the reduction is designed to prevent temperature overshoot. When at least one minute had elapsed since the addition of enzyme(s), the feed tank supply to the Moyno was turned on simultaneously as the water was turned off. A timer was also started. If the feed pressure does not settle at about 100 psi, the combining tube is adjusted to attain the desired pressure. The starch front should exit the flash tank at the calculated time. Time should be about 10-15% longer due to steam add-on and residence time, pipe turns, etc, none of which is included in the original calculation. If the temperatures are steady, a sample may be taken as early as two-hold loop time.

In general, the cook temperature is the average of the inlet and the exit loop temperatures, which should not differ from each other by more than 1.1° C. In situations where operating extremes are being tested and/or the jet is operating erratically, the exit temperature is used as the cook temperature for the sample.

After the starch cooking stage, the system was shut down by switching back to water while leaving the steam on. The temperature should go up and the feed pressure should drop back to the original. The system was flushed with hot water until the water exited clean. The steam was then turned off, and the system was cooled by operating with cold water. To remove the residual amount of starch that may hide in joints, pumps, and valves, a flush with a hypochlorite solution (CHLOROX®) can be used to reduce the stench on restart. Alternatively, the system may be flushed once a week to keep the odor down when it is not in use.

DE Determination

The DE value of a given sample was determined by the following coupled reactions. First, the sample was mixed with a known excess amount of Copper (II) ion (Cu⁺⁺). In the presence of reducing sugars (R—CHO), Copper (II) was stoichemically reduced to Copper (I) (Cu⁺). Next, the remaining Copper (II) ion was allowed to reduce the iodide ion (f) in an acidic media (^{H+}) to form the tri-iodide ion (I₃⁻). The tri-iodide ion was then titrated with a standardized thiosulfate solution (S₂O₃⁻²).

R—CHO+Cu⁺⁺ (known excess)→R—COOH+Cu⁺+Cu⁺⁺

2Cu⁺⁺+4I^{-H+}→Cu₂I₂+I₂

I₂+I⁻ [symbol] I₃⁻

I₂+2S₂O₃⁻²→2I⁻+S₄O₆⁻²

See Schoorl, N., Zurjodometrischen Zukerbestimmung mittles Fehlingscher Losung., Zeitschr. F. agnew. Chem., 12, 633 (1899); Hodge, J. E & Davis, H. A. Selected Methods for Determining Reducing Sugars, United States Department of Agriculture Technical Bulletin A1C333 (1952); and Schenck, F. W. & Hebeda, R. E., Starch Hydrolysis Products, Worldwide Technology Production and Applications, p 379, VCH Publishers (1992).

A sample dilution containing an equivalent of approximately 47-67 mg of dextrose was prepared in a 10 ml of aliquot. When the process was used for starch liquefactions, the liquefied material was weighed into a tared 50 ml volumetric flask containing 6 drops of 4 N HCl. To achieve the target glucose, 6-8 g of approximately 35% dry substance slurry was used for the 10 ml test sample. For whole grain liquefactions, it is difficult to handle ground particulate paste. To achieve the target titer difference for whole grain liquefactions, the weight used should be 1.3-1.8 g of approximately 30% dry substance slurry with 25 ml of deionized (DI) water added before adding Fehlings solution A (see below).

To start the assay, 10 ml of the sample was transferred into a 250 ml Erlenmeyer flask. The following reagents were added in order:
15 ml of distilled (DI) water;
10 ml of Fehlings solution A (69.3 g of analytical grade cupric sulfate pentahydrate (CuSO₄.5H₂O) was dissolved in 1 L DI water); and
10 ml of Fehlings solution B (346.0 g of potassium sodium tartrate tetrahydrate (KNaC₄H₄O₆.4H₂O) (Rochelle salt) and 100 g of analytical grade sodium hydroxide were dissolved in 1L DI water).

Boiling beads or chips may be added to minimize superheating. The content in the flask was mixed well and the flask was placed on a rheostat controlled electric heater. The heater was pre-adjusted so that the mixture was brought to boiling after 3 min±15 sec. The sample was kept boiling for two additional minutes. The total heating time was thus approximately 5 min. Subsequently, the flask was removed from the heater and immediately cooled to room temperature under tap water. Optionally, a water bath or ice bath may be used. After the mixture was cooled down, the following reagents were added in order:
10 ml of 30% potassium iodide solution; and
10 ml of 26% sulfuric acid solution.

The mixture was titrated immediately with the standardized 0.1 N sodium thiosulfate until the solution became pale yellow. The titration was continued, after 2 ml of the starch indicator (1% w/v) was added. The titration stopped until the blue starch iodine complex disappeared. The final titration color should be a pale-pink.

To calculate the DE value, a water blank (Twb) was determined by titrating 25 ml of DI water. Additionally, a standard dextrose titer (Ts) was determined by pipetting 5 ml of 1% dextrose standard and 5 ml of DI water into the reaction flask. The DE value was calculated as:

$$DE = \frac{( Twb - Tu ) \times 0.05 \times 100 \times 100 \%}{( Twb - Ts ) \times W \times \% DS}$$

wherein:

Twb=Titer of water blank;

Tu=Titer of unknown;

Ts=Titer of dextrose standard;

0.05=0.05 g of glucose;

W=weight in g of the unknown sample;

100=convert percent dry solid starch to dry solids starch;

100%=convert dextrose equivalent to a percentage; and

% DS=percent dry solids in sample.

An example calculation is presented below:

8.000 g of liquefact to 50 ml volumetric flask;

10 ml taken for analysis (1.600 g liquefact);

34.0% dry substance;

Twb=27.2;

Ts=12.73; and

Tu=11.00.

$$DE = \frac{( 27.2 - 12.73 ) \times 0.05 \times 100 \times 100}{( 27.2 - 11.00 ) \times 1.600 \times 34} = 8.78$$

Iodine Test

For saccharide liquor iodine test, 0.2 ml saccharide liquor was diluted with 10 ml of DI or RO water. The diluted saccharide liquor was boiled for 10 minutes and then cooled in an ice bath. 0.5 ml iodine solution (0.02 M) was added to the cooled saccharide liquor sample. The samples were allowed to stand at least 10 minutes before reading.

Sediment Test

All starches, especially grain-based, contain traces of components other than dextrose polymers such as fine fiber, proteins, fats, and ash that are released during hydrolysis. The starch cooking parameters and operating equipment such as the steam jet cooker has a bearing on the quantity of this material. Small amounts of starch-lipid complexes and under the right conditions, partially pasted and/or whole starch granules may pass through the liquefaction system. Due to incomplete hydrolysis in the liquefaction system, the most reliable location to test for these components is after complete saccharification. A well run liquefaction system that is receiving well-washed starch from the milling division should test at <1.5% sediment by this method. There are systems that consistently deliver <1%. Operating history has shown that sediment levels above 2.5% will result in down stream filtration difficulties, and thus costs for pre-coat media and/or microfilters.

This method described herein may be used for all dextrose substrates with >90% dextrose. This may also be used for maltose liquors, as well as liquefied low DE products. Due to viscosity and buoyant force issues caused by final saccharified dry substances >5%, liquors known to be greater than this should be diluted prior to testing.

Samples of saccharide liquor were incubated in a 60° C. water bath for 10-30 minutes to bring them to a constant temperature. The incubation, however, should not be longer than one hour. If necessary, the ds value was adjusted to 35%±0.5% prior to testing. For each sample, 15 ml of saccharide liquor was mixed well on a magnetic stirrer, and transferred to a centrifuge tube with a syringe. Samples were centrifuged at 2,500 rpm (1,350×g) for 10 minutes. The sediment, if present, is visible at the bottom of the centrifuge tube.

Filtrate Test for Saccharified Starch

This test is based on the filtration rate through a controlled depth of filter aid (diatomaceous earth) under controlled temperature and vacuum. This test can identify differences in liquefaction enzymes and processes, following saccharification. This test is suitable for the simulation of industrial rotary vacuum pre-coat filtration systems. It may be used for determination and demonstration of various liquefaction and saccharification enzymes and processes. In addition, the filtrate provides clean material for further evaluation such as the determination of soluble starch with iodine reaction.

Column jackets were maintained at 60° C. Two filter paper discs were inserted and screwed in the fitting until snug against the O-ring gasket. While a tared 250 ml vacuum flask was in place, 100 ml of water was added to the column with the exit plugged. The vacuum pump was turned on until a steady vacuum of 23-24 inches was achieved. The tube exit was turned on, and a timer was started. The 100 ml takes about 1 min 10 seconds to 1 min 30 seconds to filter through the system. If not, then check the papers to make sure they are tight. After the papers were pulled to dryness, the exit tube was clamped. The pump was left running with the clamp removed from the exit tube. The flask was replaced with a tared 250 ml filter flask. Approximately 2.0 g of filter aid was mixed with 100 g of test liquor in a 250 ml beaker. While the sample was stirring on the magnetic plate, a syringe was used to remove the sample with targeted quantity. A top loading balance may be used for this step. While keeping the particulates in suspension, the entire quantity was rapidly transferred to the column with the aid of a funnel. The exit tube clamp was turned on, and a timer was started. Collect until the liquor reaches the top of the filter bed and record the time. The quantity of filtrate across multiple tests may be used to judge operating differences in liquefaction or saccharification. Alternatively, the rate may be calculated in weight or volume per square meter of filter bed.

For example, 60 g of filtrated was collected in 15 minutes. The area of filter bed surface is calculated as πr², in this case 3.141593×0.75×0.75=1.767 cm² (the column has an inner radius of 0.75 cm). In addition, the 60 g of filtrate was equivalent to 52 ml of the sample, which has a 35% DS and a density of 1.151 g/mL. The filtrate rate is thus 52 ml/1.767 cm²/15 min=1.96 ml/cm²/min.

HPLC Method to Measure the Saccharide Composition

The composition of saccharification products was measured by a HPLC system (Beckman System Gold 32 Karat Fullerton, Calif.). The system, maintained at 50° C., was equipped with a Rezex 8 u8% H Monosaccharides column and a refractive index (RI) detector (ERC-7515A, Anspec Company, Inc.). Diluted sulfuric acid (0.01 N) was applied as the mobile phase at a flow rate of 0.6 ml/min. 20 μl of 4.0% solution of the reaction mixture was injected onto the column. Elution profiles were obtained over 45 minutes. The distribution of saccharides and the amount of each saccharide were determined from previously run standards.

AmyE's Transglucosidase Activity

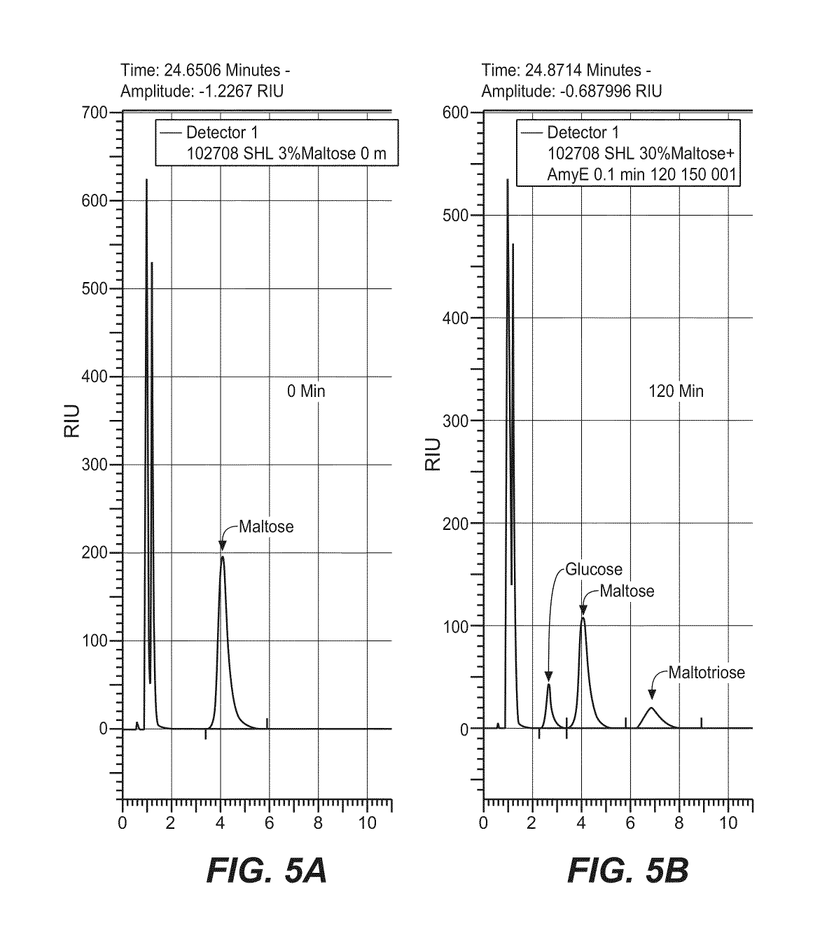

It was observed that the AmyE (SEQ ID NO: 1) is able to catalyze the formation of the tri-saccharide from maltose. This catalytic activity is likely due to AmyE's transglucosidase activity. FIG. 5 depicts the HPLC detection of the tri-saccharide after incubating AmyE with maltose. Specifically, an aliquot sample of AmyE, 0.1 ml, was added to 5 ml of 30% maltose in phosphate buffer, pH 4.5, and incubated for 60 min at 60° C. The reaction was terminated by placing the sample in a boiling water bath for 10 minutes. The reaction mixture was then subject to HPLC analysis.

### Example 3

As disclosed in U.S. patent application Ser. No. 12/478,368 (filed Jun. 4, 2009), saccharification reactions catalyzed by a glucoamylase and the AmyE resulted in, among other things, a higher level of fermentable sugars and a concomitantly reduced level of higher sugars. Furthermore, the supplementation of AmyE in the saccharification reduced the amount of IPS present in the saccharide liquor in a statistically significant manner, making it more suitable for sweetener applications. Accordingly, it would be of great interest to investigate the feasibility whether AmyE can be used as a remedy for a saccharide liquor that is found, post-saccharification, to be of poor quality and contain a high level of IPS. Presently, there is no post-saccharification method to effectively eliminate or reduce IPS by a selective enzymatic degradation.

Creation of Liquefact with Poor Quality

It is known that both Fuelzyme®-LF and straight G. stearothermophilus alpha-amylase (AmyS) produce a liquefact that begins to show iodine-positive polymers at about 24 hours into saccharification. Additionally, it is known that lowering either pH or temperature in the pilot jet may result bad liquefaction even if the secondary liquefaction results in a DE value of about 10. Accordingly, a starch was purposely processed under the above known condition(s), and the liquefied starch was saccharified without cooling below saccharification temperature.

One 45 liter batch and one 90 liter batch of corn starch slurry were prepared at 38% ds. Sulfurous acid was added to provide 100 ppm of SO₂. The first batch was adjusted to pH 4.5 with 20% sodium carbonate solution and dosed with Fuelzyme®-LF alpha-amylase (Verenium Corp.) at 50 MWU/g of dry substance starch (dss). The starch was liquefied using the pilot jet cooker (as described in Example 2). The cook temperature was about 109.3° C. with a hold time of about 6.5 to 7 minutes. A one-liter sample was placed into about a 95° C. water bath for the secondary liquefaction.

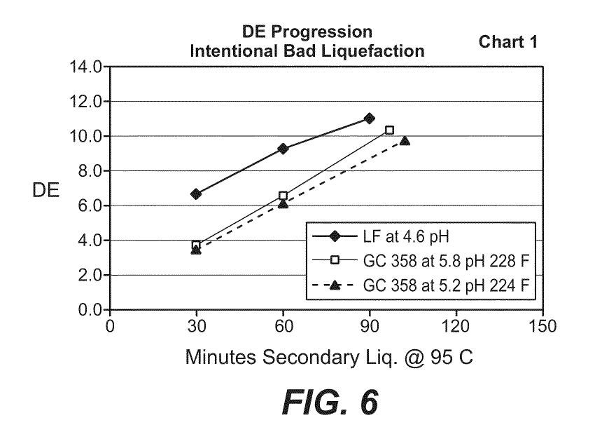

The second batch of 90 liters was adjusted to pH 5.8 and dosed with GC 358 alpha-amylase (Danisco US Inc., Genencor Division) at 1.2 AAU/g dss. The slurry was started through the cooker at about 108.5° C. A one-liter sample was taken at about 15 minutes for secondary liquefaction (“good cook”). The remaining slurry was adjusted to about pH 5.25 with HCl, and the temperature was adjusted to a target of about 106.7° C. (“poor cook”). The jet operation became unstable at a lower temperature, e.g., about 106.7° C. Samples were removed from the cook tube at an average temperature of about 105.9° C. FIG. 6 shows the DE development of the above three liquefactions. The target was to stop the secondary liquefaction at a DE value of about 10. The termination process was achieved by decreasing the pH to about 3 with HCl and keeping the samples at the secondary liquefaction temperature for an additional 20 minutes. Since Fuelzyme®-LF remains stable at lower pH conditions, the termination process was not done for the liquefaction catalyzed by Fuelzyme®-LF. The liquefaction results are also presented in Table 1. It was estimated that 50 MWU/g dss of Fuelzyme®-LF (1) made about 4.7 DE in the primary stage, and (2) displayed a typical rate of DE development in the second liquefaction, reaching 10 DE in a very short time. For GC 358, 10 DE was reached at a comparable rate even for the liquefaction that was performed under the known “poor cook” condition. The DE development alone, therefore, is not a reliable measurement for liquefaction quality.

TABLE 1
Liquefaction results

| | | | | | | min. |
|---|---|---|---|---|---|---|
| | | Temperature | | | | to 10 |
| Enzyme | pH | (° C.) | slope | intercept | correlation | DE |
| Fuelzyme ®- | 4.6 | 109.3 | 0.072 | 4.691 | 0.994 | 73 |
| LF | | | | | | |
| GC 358 | 5.8 | 108.5 | 0.099 | 0.728 | 1.000 | 94 |
| GC 358 | 5.2 | 106.7 | 0.088 | 0.899 | 1.000 | 104 |

Preliminary Characterization of Elimination/Reduction of IPS by AmyE

The above liquefaction samples were cooled to about 60° C. and subject to saccharification according to Table 2.200 g samples from each liquefact were placed into seven bottles. The dose plan was to cover the following scenarios: (1) tests 2 and 3 with AmyE used as an additive to OPTIMAX® 4060 VHP (Danisco US Inc., Genencor Division), (2) tests 4 and 5 as if it was discovered at 24 hours that a saccharide tank had a problem, and 3) tests 6 and 7 simulated the discovery of positive starch at the 48 hour point.

TABLE 2
Initial set-up for saccharification and AmyE treatment

| | | Dose | AmyE | add AmyE |
|---|---|---|---|---|
| | Test # | GAU/g | mg/g | Hr |
| Fuelzyme ®-LF | 1 | 0.16 | 0 | |
| | 2 | 0.16 | 0.05 | 0 |
| | 3 | 0.16 | 0.01 | 0 |
| | 4 | 0.16 | 0.05 | 24 |
| | 5 | 0.16 | 0.01 | 24 |
| | 6 | 0.16 | 0.05 | 48 |
| | 7 | 0.16 | 0.01 | 48 |
| GC 358 good cook | 1 | 0.16 | 0 | |
| | 2 | 0.16 | 0.05 | 0 |
| | 3 | 0.16 | 0.01 | 0 |
| | 4 | 0.16 | 0.05 | 24 |
| | 5 | 0.16 | 0.01 | 24 |
| | 6 | 0.16 | 0.05 | 48 |
| | 7 | 0.16 | 0.01 | 48 |
| GC 358 bad cook | 1 | 0.16 | 0 | |
| | 2 | 0.16 | 0.05 | 0 |
| | 3 | 0.16 | 0.01 | 0 |
| | 4 | 0.16 | 0.05 | 24 |
| | 5 | 0.16 | 0.01 | 24 |
| | 6 | 0.16 | 0.05 | 48 |
| | 7 | 0.16 | 0.01 | 48 |

The above plan was changed at the 48-hour time point due to the complete lack of any notable iodine color reduction for samples 2-5 at 48 hour point. The AmyE dose was adjusted to 0.2 or 0.8 mg/g. Additionally, sample 1 for each series was dosed with 0.2 mg/g AmyE and placed into a 32° C. water bath. At 120 hours, the pH value of samples 4 and 5 in each series were adjusted to 5.2 with sodium carbonate. Sample 4 in each series was re-dosed with AmyE at 0.8 mg/g; while sample 5 in each series was re-dosed with AmyE at 0.8 mg/g and phytase BP-17 at 1 FTU/g. The summary of the adjustment is shown in Table 3.

TABLE 3
Modified set-up for AmyE treatment

AmyE dose (mg/g dss) and timing

| Test # | 0 hr | 24 hr | 48 hr | 120 hr |
|---|---|---|---|---|
| 1 | | | 0.2/32° C. | |
| 2 | 0.05 | | | |
| 3 | 0.01 | | | |
| 4 | | 0.05 | | 0.8/pH 5.2 |
| 5 | | 0.01 | | 0.8/1 FTU/g/pH 5.2 |
| 6 | | | 0.2 | |
| 7 | | | 0.8 | |

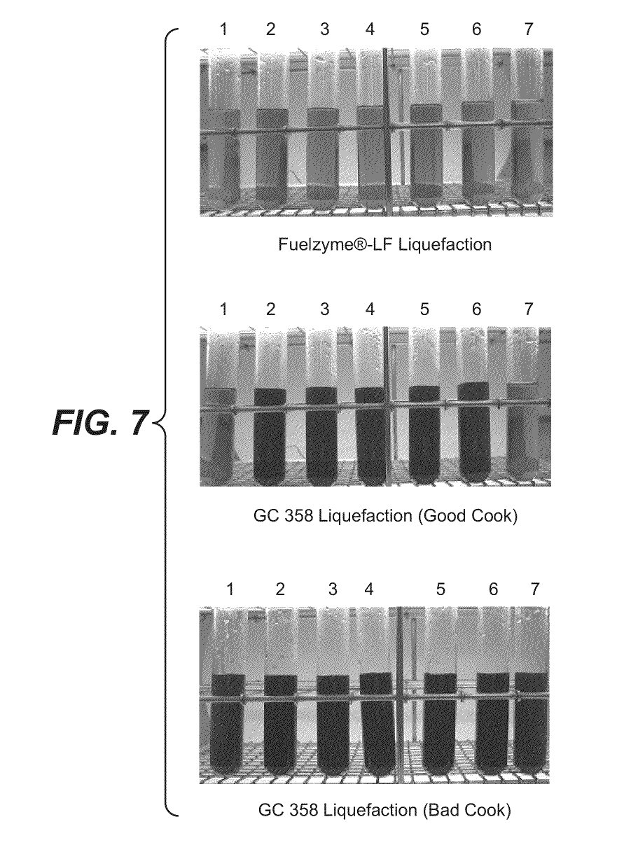

Samples were subject to iodine color test at various time points. The removal of IPS can be visually inspected based on the color. The results of the iodine color test at 112 hour time point are present in FIG. 7. None of the samples at this time point would be considered negative, though samples from the Fuelzyme®-LF liquefact looked relatively good and clearer. Previous experiments, however, indicated that the Fuelzyme®-LF liquefact could be worse absent the addition of AmyE. Additionally, it is noted that Fuelzyme®-LF was not inactivated, because it is stable under lower pH conditions. Accordingly, Fuelzyme®-LF likely remained active during saccharification and the subsequent treatment, resulting relatively improved colors.

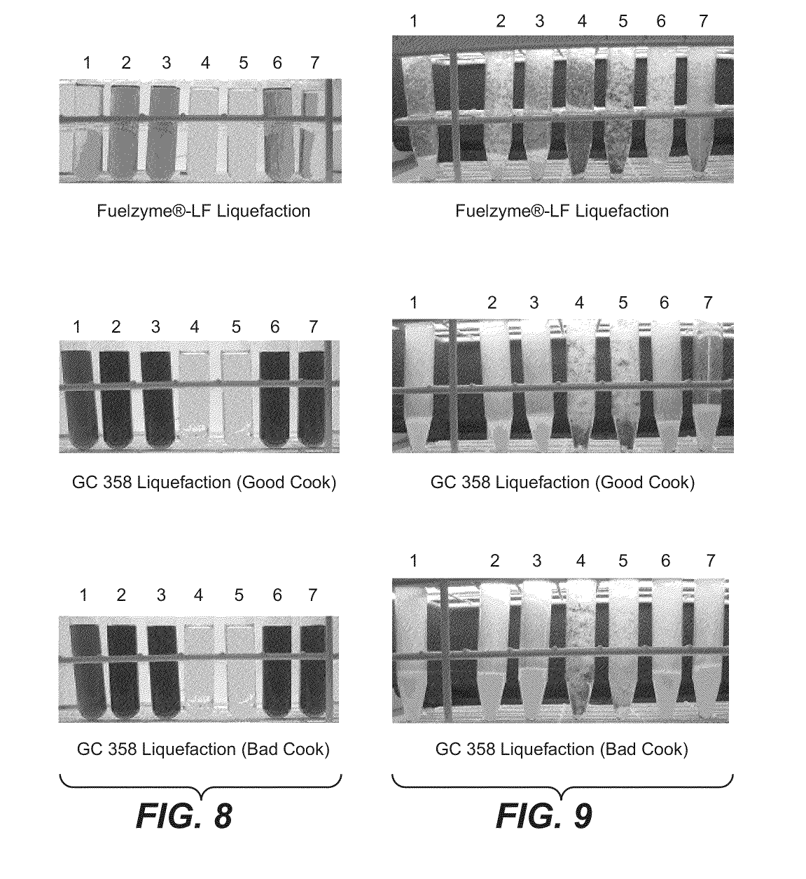

The results of the iodine color test at about the 136 hour time point are present in FIG. 8. Tube 1 in each series is the continuation of 0.2 mg/g AmyE added at the 48 hour point and held at 32° C. Tubers 4 and 5 in each series have been treated by 0.8 mg/g of AmyE for about 16 hours at 60° C., pH 5.2. It is obvious that AmyE at a dose of 0.8 mg/g is unable to remove the iodine-positive starch if the pH is kept between about 4 and about 4.5. Once the pH is increased to about 5.2, however, complete removal of the iodine-positive starch is possible.

Furthermore, sediment test (as described in Example 2) was performed at 136 hour point for all samples. The results are presented in FIG. 9. Although the samples from the Fuelzyme®-LF liquefact displayed better iodine colors than the samples from the GC 358 liquefacts, there remained substantial quantities of sediment in samples from the Fuelzyme®-LF liquefact. Sediment in tubes 4 and 5 in each series was reduced to a statistically significant amount, indicating that AmyE treatment almost resulted in the elimination of the sediment. The reduction of sediment by a high dose of AmyE (0.8 mg/g) at pH 5.2 was the most significant in all series, i.e., from (1) about 7% to almost 0% for samples of the Fuelzyme®-LF liquefact, (2) about 8% to almost 0% for samples of the GC 358 (good cook), and (3) about 17% to almost 0% for samples of the GC 358 (bad cook). At the saccharification pH, i.e., 4.0-4.5, treatment by 0.8 mg/g AmyE resulted in a slight, though noticeable, reduction of both the iodine color and the sediment level. Additionally, although tube 1 (held at 32° C. for AmyE treatment) displayed reduced iodine color in all series, it still had the same quantity of sludge, suggesting that reduction of iodine-positive starch does not necessarily correlate with reduction of sediment.

The filtration rates were determined as described in Example 2 for tests 2 and 4 in each series. A comparison indicated that AmyE treatment is capable of improving filtration for a saccharide liquor resulted from a poorly liquefied starch. For samples from the Fuelzyme®-LF liquefact, the filtration rate improved from 48 to 60 g, representing a 25% improvement. For samples from the GC 358 liquefact (good cook), an improvement from 35 to 47 g, i.e., about 34%, was observed. The most significant improvement was observed for samples from the GC 358 liquefact (good cook), the filtration rate improved from 18 to 55 g, reflecting a significant increase in the filtration rate of about 200%.

For the AmyE to be an enzyme usable to remedy the blue saccharide, the saccharide composition in a saccharide liquor should not dramatically change because of AmyE treatment. Accordingly, the saccharide composition was determined at 24 hr and 48 hr time points for all samples and presented in Table 4. The determination of oligosaccharide was achieved by the HPLC method as described in Example 2.

TABLE 4
Saccharide distributions

| | Series A | | | | | | | | | | | | | | |
|---|---|---|---|---|---|---|---|---|---|---|---|---|---|---|---|
| | LF liq | hrs | DP1 | DP2 | DP3 | DP4 | DP5 | DP6 | DP7 | DPS8 | DP9 | DP10 | DP10+ | [symbol] | DP 4+ |
| AmyE addtions | | | | | | | | | | | | | | | |
| in mg of protien/mg ds | | | | | | | | | | | | | | | |
| Control | 1 | 24 | 86.05 | 3.69 | 3.95 | 1.97 | 0.77 | 0.78 | 0.64 | 0.64 | 1.51 | | | 93.70 | 6.30 |
| | | 48 | 92.89 | 2.18 | 2.62 | 0.54 | 0.26 | 0.31 | 0.26 | | 0.94 | | | 97.69 | 2.31 |
| +0.05 at 0 time | 2 | 24 | 84.02 | 3.74 | 4.01 | 2.02 | 0.88 | 1.29 | 1.61 | 1.01 | 1.42 | | | 91.77 | 8.23 |
| | | 48 | 94.93 | 2.63 | 1.80 | | | | 0.25 | | | 0.36 | | 99.39 | 0.62 |
| +0.01 at 0 time | 3 | 24 | 83.83 | 3.91 | 4.37 | 2.40 | 1.16 | 1.06 | 0.81 | 0.86 | 1.61 | | | 92.11 | 7.90 |
| | | 48 | 93.85 | 2.52 | 2.74 | | | 0.09 | | | 0.43 | | | 99.11 | 0.52 |
| +0.05 at 24 hr | 4 | 24 | 86.16 | 3.77 | 3.95 | 1.96 | 0.79 | 0.79 | 0.61 | 0.61 | 1.36 | | | 93.87 | 6.13 |
| | | 48 | 93.69 | 2.31 | 2.48 | 0.30 | | 0.16 | 0.18 | | 0.89 | | | 98.48 | 1.52 |
| +0.01 at 24 hour | 5 | 24 | 85.84 | 3.76 | 4.08 | 2.08 | 0.78 | 0.75 | 0.66 | 0.85 | 1.21 | | | 93.67 | 6.33 |
| | | 48 | 93.32 | 2.27 | 2.58 | 0.32 | 0.10 | 0.22 | 0.22 | | 0.97 | | | 98.17 | 1.83 |
| +0.2 at 48 hr | 6 | 24 | 84.62 | 4.51 | 4.55 | 2.22 | 0.93 | 0.88 | 0.66 | 0.69 | 0.94 | | | 93.67 | 6.33 |
| | | 48 | 94.66 | 2.54 | 1.34 | | | 0.11 | 0.20 | | 0.89 | | | 98.54 | 1.20 |
| +0.8 at hr | 7 | 24 | 85.97 | 3.66 | 3.94 | 1.97 | 0.74 | 0.80 | 0.69 | 0.73 | 1.51 | | | 93.57 | 6.43 |
| | | 48 | 93.56 | 2.15 | 2.46 | | 0.12 | 0.23 | 0.22 | | 0.93 | | | 98.17 | 1.50 |
| Series B GC 358 | | | | | | | | | | | | | | | |
| “good” | | | | | | | | | | | | | | | |
| Control | 1 | 24 | 87.28 | 2.80 | 0.58 | 0.40 | 0.43 | 0.65 | 0.74 | 0.82 | 0.63 | 0.41 | 5.26 | 90.65 | 9.35 |
| | | 48 | 95.53 | 1.91 | 0.46 | | | 0.13 | 0.52 | 1.34 | | | | 97.89 | 1.99 |
| +0.05 at 0 time | 2 | 24 | 86.39 | 7.41 | 1.58 | 1.25 | 1.07 | 0.34 | 0.37 | 1.47 | | | 0.14 | 95.37 | 4.63 |
| | | 48 | 92.83 | 4.64 | 0.84 | 0.11 | | 0.14 | 0.30 | 1.13 | | | | 98.32 | 1.68 |
| +0.01 at 0 time | 3 | 24 | 85.36 | 7.04 | 1.48 | 1.03 | 1.62 | 0.39 | 0.40 | 1.59 | | | 1.10 | 93.87 | 6.13 |
| | | 48 | 94.01 | 3.45 | 0.73 | | | 0.12 | 0.30 | 1.25 | | | 0.14 | 98.20 | 1.80 |
| +0.05 at 24 hr | 4 | 24 | 87.18 | 2.85 | 0.60 | 0.43 | 0.46 | 0.68 | 0.73 | 0.72 | 0.71 | 0.44 | 4.92 | 90.63 | 9.08 |
| | | 48 | 95.50 | 2.32 | 0.51 | | | | 0.35 | 1.19 | | | | 98.33 | 1.53 |
| +0.01 at 24 hour | 5 | 24 | 89.77 | 3.15 | 0.59 | 0.39 | 0.41 | 0.45 | 0.35 | 0.31 | 0.25 | 0.13 | 4.20 | 93.52 | 6.48 |
| | | 48 | 95.60 | 2.22 | 0.50 | | | 0.11 | 0.32 | 1.26 | | | | 98.31 | 1.68 |
| +0.2 at 48 hr | 6 | 24 | 86.86 | 2.85 | 0.59 | 0.42 | 0.47 | 0.73 | 0.79 | 0.76 | 0.74 | 0.84 | 4.96 | 90.30 | 9.70 |
| | | 48 | 95.51 | 2.22 | 0.45 | | | 0.11 | 0.36 | 1.26 | | | 0.10 | 98.17 | 1.83 |
| +0.8 at 43 hr | 7 | 24 | 82.68 | 2.59 | 0.63 | 0.45 | 0.48 | 0.69 | 0.76 | 0.96 | 0.90 | 1.48 | 8.40 | 85.89 | 14.11 |
| | | 48 | 95.36 | 2.14 | 0.62 | | | 0.12 | 0.33 | 1.27 | | | | 98.13 | 1.72 |
| Series C GC 358 [bad] | | | | | | | | | | | | | | | |
| Control | 1 | 24 | 90.73 | 2.66 | 0.60 | 0.40 | 0.39 | 0.45 | 0.37 | 0.34 | 0.31 | 0.12 | 3.62 | 94.00 | 6.003 |
| | | 48 | 95.57 | 2.17 | 0.45 | | | 0.13 | 0.38 | 1.30 | | | | 98.19 | 1.81 |
| +0.05 at 0 time | 2 | 24 | 85.56 | 7.09 | 1.76 | 1.31 | 1.12 | 0.42 | 2.55 | | | | 0.20 | 94.41 | 5.59 |
| | | 48 | 93.32 | 4.18 | 0.85 | | | 0.14 | 0.26 | 1.14 | | | | 98.36 | 1.54 |
| +0.01 at 0 time | 3 | 24 | 87.02 | 6.36 | 2.15 | 0.85 | 1.34 | | 0.25 | | 0.26 | 1.04 | 0.75 | 95.52 | 4.48 |
| | | 48 | 94.85 | 2.84 | 0.71 | | | 0.13 | 0.26 | 1.20 | | | | 98.41 | 1.60 |
| +0.05 at 24 hr | 4 | 24 | 90.72 | 2.66 | 0.86 | 0.42 | 0.39 | | 0.37 | | 0.43 | 0.32 | 3.85 | 94.23 | 5.77 |
| | | 48 | 95.46 | 2.25 | 0.47 | | | 0.11 | 0.35 | 1.26 | | | 0.10 | 98.19 | 1.82 |
| +0.01 at 24 hour | 5 | 24 | 87.91 | 2.53 | 0.64 | 0.37 | 0.48 | 0.70 | 0.76 | 0.72 | 0.76 | 0.93 | 4.20 | 91.08 | 8.82 |
| | | 48 | 95.61 | 2.24 | 0.46 | | | 0.11 | 0.33 | 1.25 | | | | 98.31 | 1.69 |
| +0.2 at 48 hr | 6 | 24 | 87.93 | 2.66 | 0.59 | 0.45 | 0.49 | 0.68 | 0.74 | 0.70 | 0.73 | 0.74 | 4.30 | 91.18 | 8.81 |
| | | 48 | 95.36 | 2.80 | 0.39 | | | | 0.27 | | 1.05 | | 0.14 | 98.55 | 1.46 |
| +0.8 at 48 hr | 7 | 24 | 91.77 | 1.70 | 0.25 | 0.30 | 0.66 | 0.45 | 0.60 | 0.61 | 0.42 | | 2.85 | 93.72 | 5.90 |
| | | 48 | 95.30 | 2.84 | 0.38 | | | 0.10 | 0.28 | | 0.99 | | 0.11 | 98.52 | 1.48 |

[symbol]

indicates data missing or illegible when filed

In Table 4, tests 1, 6, and 7 are in fact duplicates, because tests 6 and 7 were dosed with additional AmyE at 48 hour time point after the sample was taken. For samples from GC 358 liquefact with AmyE added simultaneously with the glucoamylase (0 hour time point), both DP2 and DP3 were higher, while DP1 was lower. This does not seem to be the case when AmyE is added after 24 hours of saccharification, however, because DP1 was at a level of about 87% or higher. Table 4 also shows the impact of using Fuelzyme®-LF for liquefaction and subsequent high glucose production: DP1 was low, DP2 was about normal, and DP3 was about 2% absolute higher than samples from the GC 358 liquefact. The observed difference likely attributes to the fact that GC 358 was inactivated by lowering the pH, while Fuelzyme®-LF remained active. Overall, the addition of AmyE at 0 time produced the highest level of fermentable sugars (DP1, DP2, and DP3 altogether).

### Example 4

It was observed in Example 3 that AmyE is effective for the complete removal of iodine-positive starch when (1) AmyE is dosed at a high level, and (2) pH is adjusted at the end of saccharification from about 4.1 to about 5.0-5.2. Additionally, AmyE at a high dose is also able to remove high quantities of sediment and improve filtration rates. Nevertheless, the removal of iodine-positive starch is not completed as observed in Example 3, and the saccharide distribution is slightly affected. Further optimization can be necessary for the AmyE-mediated IPS removal.

The starch liquefied with GC 358 (straight B. stearothermophilus alpha-amylase) at 109° C. and pH 5.8 was stored frozen. It was warmed to room temperature, and pH was adjusted to 4.5 using sodium carbonate. The liquefact was dosed with OPTIMAX® 4060 VHP at 0.16 GAU/g of dry substance. The dry substance was about 34.5%. It is known that GC 358 pilot jet liquefied starch will contain iodine positive polymers that become detectable after about 24 hours of saccharification, and will have a high level of sediment at the end of saccharification. Additionally, this liquefact contained retrograded starch, because it was frozen and then thawed.

After 42 hours of saccharification at 60° C., the portion for additional saccharification with AmyE or Clarase® L (a fungal alpha-amylase from Danisco US Inc., Genencor Division) was adjusted to about pH 5.3 with sodium carbonate. The portion for additional saccharification with G-ZYME® G 998 (an aciduric amylase from Danisco US Inc., Genencor Division) was left at about pH 4.2 without adjustment. Each 100 g aliquot was placed into a 250 ml bottle and dosed as shown in Table 5.

TABLE 5
Set-up for saccharification and subsequent alpha-amylase treatment

| | | A | B | C |
|---|---|---|---|---|
| | Dose | AmyE | Clarase | G 998 |
| Test # | GAU/g | mg/g | kg/mt | kg/mt |
| 1 | 0.16 | 0.4 | 1 | 2 |
| 2 | 0.16 | 0.1 | 0.3 | 0.5 |
| 3 | 0.16 | 0.05 | 0.1 | 0.1 |
| 4 | 0.16 | 0.01 | 0.05 | 0.05 |
| 5 | 0.16 | 0 | 0 | 0 |

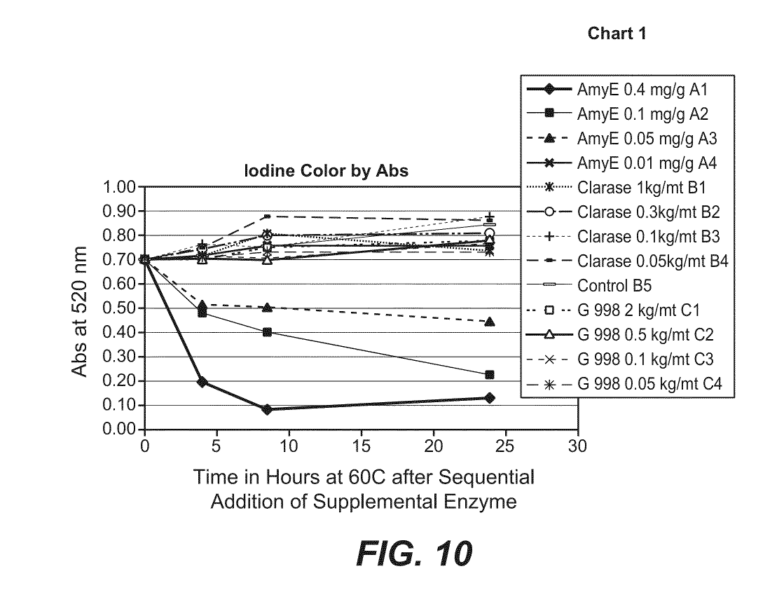

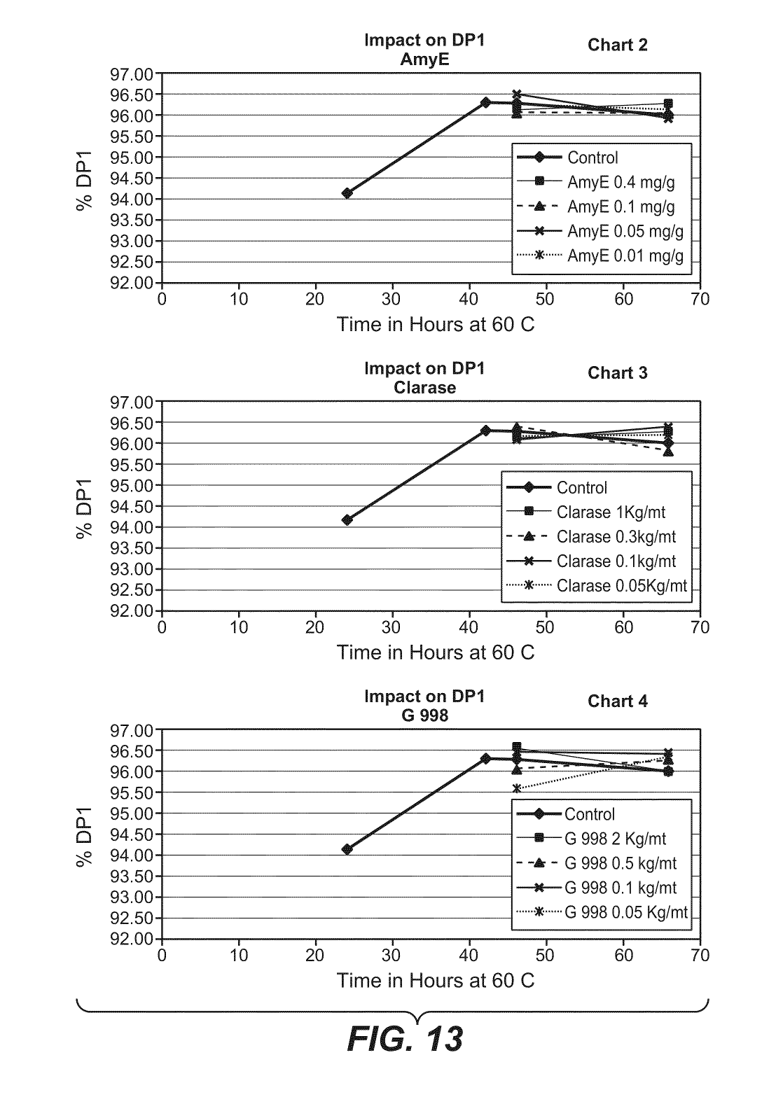

The samples were replaced into the 60° C. water bath and samples were collected at 4 hours for carbohydrate distribution analysis and iodine color test, at 8.5 hours for iodine color test, and at 24 hours for carbohydrate distribution analysis and iodine color test. The results of iodine color are compiled in Table 6 and presented in FIGS. 10-12.

TABLE 6
OD_{520 nm} reading of various treated samples

| Samples | | 4 hr | 8.5 hr | 24 hr |
|---|---|---|---|---|
| 0.4 mg/g AmyE | A1 | 0.197 | 0.082 | 0.130 |
| 0.1 mg/g AmyE | A2 | 0.475 | 0.395 | 0.223 |
| 0.05 mg/g AmyE | A3 | 0.515 | 0.505 | 0.446 |
| 0.01 mg/g AmyE | A4 | 0.713 | 0.757 | 0.755 |
| 1 kg/mt Clarase | B1 | 0.715 | 0.802 | 0.736 |
| 0.3 kg/mt Clarase | B2 | 0.739 | 0.794 | 0.806 |
| 0.1 kg/mt Clarase | B3 | 0.760 | 0.750 | 0.875 |
| 0.05 kg/mt Clarase | B4 | 0.750 | 0.873 | 0.859 |
| Control | B5 | 0.718 | 0.747 | 0.840 |
| 2 kg/mt G 998 | C1 | 0.697 | 0.741 | 0.770 |
| 0.5 kg/mt G 998 | C2 | 0.700 | 0.698 | 0.783 |
| 0.1 kg/mt G 998 | C3 | 0.708 | 0.703 | 0.762 |
| 0.05 kg/mt G 998 | C4 | 0.708 | 0.731 | 0.731 |

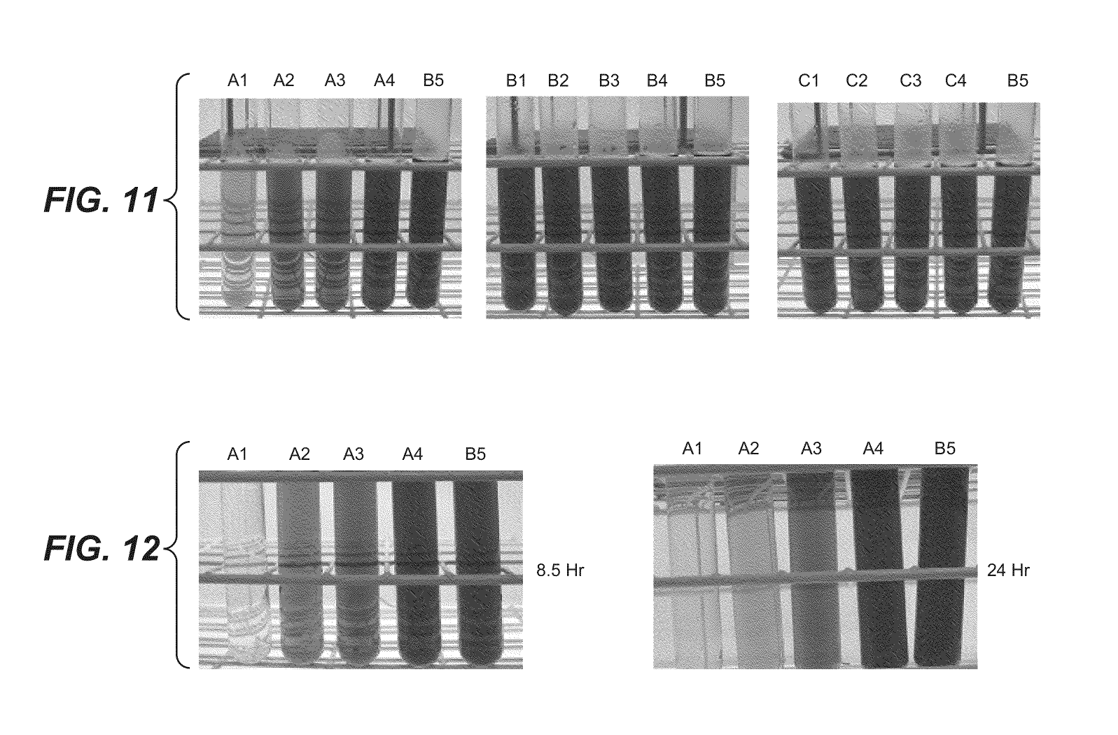

The control with no additional enzyme, B5, showed increasing iodine color development over time. This observation is consistent with what has been known that the iodine color keeps developing as the saccharification continues. Any improvement of iodine color was only observed for AmyE-containing samples, as neither Clarase® L nor G-ZYME® G 998 was able to reduce the iodine color even at high doses. The reduction of iodine color indicated that AmyE effectively reduces the polymers responsible for binding iodine. It was noted that the samples contained residual particulate as they are from whole saccharide liquor. This is probably the explanation of the 24 hour reading for sample A-1, which indicated a “false” increase, while the color as shown in FIG. 11 remained yellow. As shown in FIG. 11, 4-hour treatment by 0.4 mg/g AmyE resulted in the removal of, if not all, most starch polymers that would stain blue with iodine. The color of A1 (0.4 mg/g AmyE) at 4 hours may be judged as lightly positive in a wet milling plant. The color of A1 (0.4 mg/g AmyE) at 8.5 hour point was completely yellow, and likely would be judged to be negative in any plant. The color of A2 (0.1 mg/g AmyE) at 8.5 hour point indicated removal of significant quantities of IPS, even though it remained positive. After 24 hours, the color of A2 (0.1 mg/g AmyE) indicated the removal of most starch polymers. The color of A3 (0.05 mg/g AmyE) at 24 hour time point was noticeably improved, though its rate of IPS removal may not be practical for industrial applications.

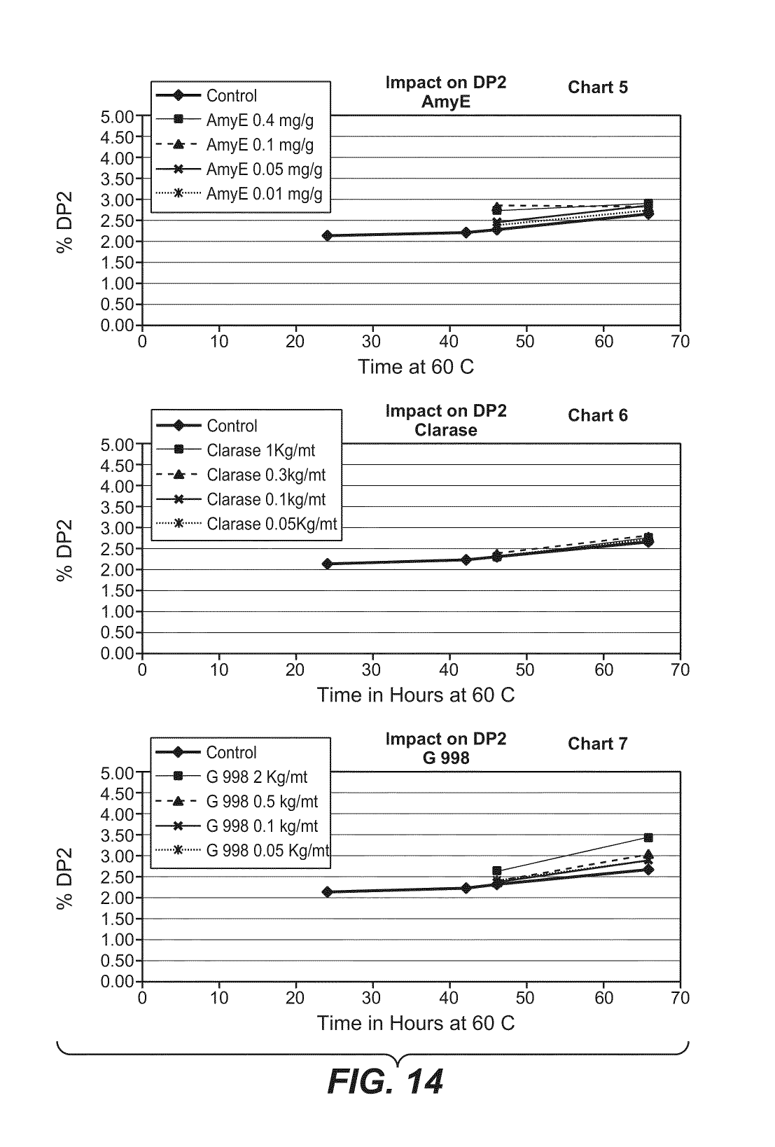

One of the issues for elimination/reduction of iodine positive starch is the possible major loss of glucose during an enzyme-mediated removal process. Possible condensation products especially isomaltose from glucoamylase reversions is always an issue. As shown in Example 2 and FIG. 5, AmyE is capable of synthesizing maltotriose from maltose because of its transglucosidase activity. It is expected that AmyE treatment may result in a decrease in maltose level, an increase in DP3 and 4 levels, and a slight increase in DP1 level. The saccharide distribution data are presented in Table 7 and FIGS. 13-14.

As shown in FIGS. 13-14, AmyE treatment at various doses resulted in very little change or loss in the DP1 level. The impact on DP2 shown in FIG. 14, however, indicated that the DP2 level increased dramatically at the 4 hour time point, probably due to reduction of higher sugars. Subsequently, the DP2 level remained nearly constant and was merely slightly lower than that of the control, which contained only OPTIMAX® 4060 VHP without any amylase.

Compared to the control, the Clarase® L treated samples showed a diminished DP1 loss, which may result from the pH shift. The DP2 for the Clarase® L treated samples was at a similar level as that of the control. For the G-ZYME® G 998 treated samples, the high dose (2 Kg/mt) showed an increase in DP1 at 4 hours and then a loss at the 24 hour point. The G-ZYME® G 998 treatment at lower doses (0.5. 0.1 and 0.01 kg/mt), however, indicated that the DP1 level actually increased slightly. Additionally, G-ZYME® G 998 treatment resulted in an increase of DP2, particularly when G-ZYME® G 998 was dosed at a high level, e.g., 2 Kg/mt. It appeared that G-ZYME® G 998 was making mostly DP2 from the remaining higher sugars. Although all the G-ZYME® G 998 tests had the lowest level of DP4+ for any of the tests, G-ZYME® G 998 treatment showed no impact on the removal of the iodine positive polymers.

The liquefact used in the above tests is known to contain iodine positive polymers as well as retrograded starch. The amount of amylose (DP>40) that stains blue with iodine was not quantified in these tests. It is possible that there is a dose response relative to the quantity of the polymer being hydrolyzed by AmyE. If that is the case, the real application of this remedy may require a lower dose of AmyE.

TABLE 7
Saccharide distribution.

| Treatment | | Total Hours | DP1 | DP2 | DP3 | DP4 | DP5 | DP6 | DP7 | DP8 | DP9 | DP10 | >DP10 |
|---|---|---|---|---|---|---|---|---|---|---|---|---|---|
| Control | | 24 | 94.18 | 2.17 | 0.64 | 0.24 | 027 | 0.38 | 0.53 | 0.50 | 0.54 | | 0.54 |
| Control | | 42 | 96.29 | 2.25 | 0.47 | | | 0.09 | 0.26 | 0.47 | | | 0.17 |
| AmyE 0.4 | 4 hr | 46 | 96.10 | 2.75 | 0.59 | 0.13 | | | 0.27 | | | | 0.17 |
| AmyE 0.1 | 4 hr | 46 | 96.02 | 2.86 | 0.74 | | | | 0.23 | | | | 0.15 |
| AmyE 0.05 | 4 hr | 46 | 96.48 | 2.46 | 0.56 | | | | 0.21 | 0.09 | | | 0.20 |
| Amye 0.01 | 4 hr | 46 | 96.24 | 2.41 | 0.55 | | | | 0.28 | 0.22 | 0.14 | | 0.17 |
| Clarase 1 kg/mt | 4 hr | 46 | 96.13 | 2.34 | 0.52 | 0.09 | | 0.09 | 0.31 | 0.21 | 0.17 | | 0.15 |
| Clarase 0.3 kg/mt | 4 hr | 46 | 96.40 | 2.39 | 0.45 | | | | 0.28 | 0.22 | | 0.10 | 0.18 |
| Clarase 0.1 kg/mt | 4 hr | 46 | 96.10 | 2.34 | 0.54 | | | 0.13 | 0.32 | 0.25 | 0.18 | | 0.14 |
| Clarase 0.05 kg/mt | 4 hr | 46 | 96.22 | 2.33 | 0.45 | | | 0.11 | 0.29 | 0.25 | 0.19 | | 0.17 |
| Control | 4 hr | 46 | 96.28 | 2.32 | 0.47 | | | 0.10 | 0.24 | 0.22 | 0.20 | | 0.15 |
| G 998 2 kg/mt | 4 hr | 46 | 96.55 | 2.65 | 0.54 | | | | 0.10 | | | | 0.17 |
| G 998 0.5 kg/mt | 4 hr | 46 | 96.07 | 2.37 | 0.52 | | | 0.15 | 0.31 | 0.40 | | | 0.18 |
| G 998 0.1 kg/mt | 4 hr | 46 | 96.44 | 2.38 | 0.48 | | | | 0.21 | 0.19 | 0.14 | | 0.16 |
| G 998 0.05 kg/mt | 4 hr | 46 | 95.57 | 2.40 | 0.58 | 0.12 | 0.14 | 0.23 | 0.36 | 0.45 | | | 0.17 |
| AmyE 0.4 | 24 hr | 66 | 96.28 | 2.90 | 0.45 | | | | 0.14 | | | | 0.24 |
| AmyE O.1 | 24 hr | 66 | 96.05 | 2.87 | 0.45 | 0.08 | | 0.11 | 0.23 | | | | 0.22 |
| AmyE 0.05 | 24 hr | 66 | 95.92 | 2.89 | 0.64 | 0.09 | | 0.09 | 0.19 | | | | 0.18 |
| Amye 0.01 | 24 hr | 66 | 96.11 | 2.77 | 0.42 | | | 0.09 | 0.19 | 0.26 | | | 0.17 |
| Clarase 1 kg/mt | 24 hr | 66 | 96.29 | 2.76 | 0.46 | | | 0.08 | 0.19 | | | | 0.21 |
| Clarase 0.3 kg/mt | 24 hr | 66 | 95.80 | 2.82 | 0.54 | | | 0.09 | 0.24 | 0.29 | | | 0.22 |
| Clarase 0.1 kg/mt | 24 hr | 66 | 96.39 | 2.70 | 0.48 | | | | 0.18 | 0.09 | | | 0.16 |
| Clarase 0.05 kg/mt | 24 hr | 66 | 96.19 | 2.73 | 0.42 | | | | 0.20 | 0.15 | 0.11 | | 0.20 |
| Control | 24 hr | 66 | 95.98 | 2.68 | 0.45 | | | 0.10 | 0.24 | 0.37 | | | 0.19 |
| G 998 2 kg/mt | 24 hr | 66 | 95.95 | 3.48 | 0.37 | | | | | | | | 0.21 |
| G 998 05 kg/mt | 24 hr | 66 | 96.23 | 3.06 | 0.44 | | | | 0.13 | | | | 0.15 |
| G 998 0.1 kg/mt | 24 hr | 66 | 96.40 | 2.91 | 0.39 | | | | 0.12 | | | | 0.19 |
| G 998 0.05 kg/mt | 24 hr | 66 | 96.31 | 2.91 | 0.49 | | | | 0.12 | | | | 0.17 |

SEQUENCE LISTING

```
SEQ ID NO: 1: Full length Bacillus subtilis AmyE amino acid sequence.
The native signal sequence is not shown.
    1 LTAPSIKSGT ILHAWNWSFN TLKHNMKDIH DAGYTAIQTS PINQVKEGNQ
  51 GDKSMSNWYW LYQPTSYQIG NRYLGTEQEF KEMCAAAEEY GIKVIVDAVI
101 NHTTSDYAAI SNEVKSIPNW THGNTQIKNW SDRWDVTQNS LLGLYDWNTQ
151 NTQVQSYLKR FLDRALNDGA DGFRFDAAKH IELPDDGSYG SQFWPNITNT
201 SAEFQYGEIL QDSASRDAAY ANYMDVTASN YGHSIRSALK NRNLGVSNIS
251 HYASDVSADK LVTWVESHDT YANDDEESTW MSDDDIRLGW AVIASRSGST
301 PLFFSRPEGG GNGVRFPGKS QIGDRGSALF EDQAITAVNR FHNVMAGQPE
351 ELSNPNGNNQ IFMNQRGSHG VVLANAGSSS VSINTATKLP DGRYDNKAGA
401 GSFQVNDGKL TGTINARSVA VLYPDDIAKA PHVFLENYKT GVTHSFNDQL
451 TITLRADANT TKAVYQINNG PETAFKDGDQ FTIGKGDPFG KTYTIMLKGT
501 NSDGVTRTEK YSFVKRDPAS AKTIGYQNPN HWSQVNAYIY KHDGSRVIEL
551 TGSWPGKPMT KNADGIYTLT LPADTDTTNA KVIFNNGSAQ VPGQNQPGFD
601 YVLNGLYNDS GLSGSLPH
SEQ ID NO: 2: Nucleotide sequence encoding the AmyE of SEQ ID NO: 1.
CTTACAGCACCGTCGATCAAAAGCGGAACCATTCTTCATGCATGGAATTGGTCGTTCAATAC
GTTAAAACACAATATGAAGGATATTCATGATGCAGGATATACAGCCATTCAGACATCTCCGA
TTAACCAAGTAAAGGAAGGGAATCAAGGAGATAAAAGCATGTCGAACTGGTACTGGCTGTAT
CAGCCGACATCGTATCAAATTGGCAACCGTTACTTAGGTACTGAACAAGAATTTAAAGAAAT
GTGTGCAGCCGCTGAAGAATATGGCATAAAGGTCATTGTTGACGCGGTCATCAATCATACCA
CCAGTGATTATGCCGCGATTTCCAATGAGGTTAAGAGTATTCCAAACTGGACACATGGAAAC
ACACAAATTAAAAACTGGTCTGATCGATGGGATGTCACGCAGAATTCATTGCTCGGGCTGTA
TGACTGGAATACACAAAATACACAAGTACAGTCCTATCTGAAACGGTTCTTAGACAGGGCAT
TGAATGACGGGGCAGACGGTTTTCGATTTGATGCCGCCAAACATATAGAGCTTCCAGATGAT
GGCAGTTACGGCAGTCAATTTTGGCCGAATATCACAAATACATCAGCAGAGTTCCAATACGG
AGAAATCCTTCAGGATAGTGCCTCCAGAGATGCTGCATATGCGAATTATATGGATGTGACAG
CGTCTAACTATGGGCATTCCATAAGGTCCGCTTTAAAGAATCGTAATCTGGGCGTGTCGAAT
ATCTCCCACTATGCATCTGATGTGTCTGCGGACAAGCTAGTGACATGGGTAGAGTCGCATGA
TACGTATGCCAATGATGATGAAGAGTCGACATGGATGAGCGATGATGATATCCGTTTAGGCT
GGGCGGTGATAGCTTCTCGTTCAGGCAGTACGCCTCTTTTCTTTTCCAGACCTGAGGGAGGC
GGAAATGGTGTGAGGTTCCCGGGGAAAAGCCAAATAGGCGATCGCGGGAGTGCTTTATTTGA
AGATCAGGCTATCACTGCGGTCAATAGATTTCACAATGTGATGGCTGGACAGCCTGAGGAAC
TCTCGAACCCGAATGGAAACAACCAGATATTTATGAATCAGCGCGGCTCACATGGCGTTGTG
CTGGCAAATGCAGGTTCATCCTCTGTCTCTATCAATACGGCAACAAAATTGCCTGATGGCAG
GTATGACAATAAAGCTGGAGCGGGTTCATTTCAAGTGAACGATGGTAAACTGACAGGCACGA
TCAATGCCAGGTCTGTAGCTGTGCTTTATCCTGATGATATTGCAAAAGCGCCTCATGTTTTC
CTTGAGAATTACAAAACAGGTGTAACACATTCTTTCAATGATCAACTGACGATTACCTTGCG
TGCAGATGCGAATACAACAAAAGCCGTTTATCAAATCAATAATGGACCAGAGACGGCGTTTA
AGGATGGAGATCAATTCACAATCGGAAAAGGAGATCCATTTGGCAAAACATACACCATCATG
TTAAAAGGAACGAACAGTGATGGTGTAACGAGGACCGAGAAATACAGTTTTGTTAAAAGAGA
TCCAGCGTCGGCCAAAACCATCGGCTATCAAAATCCGAATCATTGGAGCCAGGTAAATGCTT
ATATCTATAAACATGATGGGAGCCGAGTAATTGAATTGACCGGATCTTGGCCTGGAAAACCA
ATGACTAAAAATGCAGACGGAATTTACACGCTGACGCTGCCTGCGGACACGGATACAACCAA
CGCAAAAGTGATTTTTAATAATGGCAGCGCCCAAGTGCCCGGTCAGAATCAGCCTGGCTTTG
ATTACGTGCTAAATGGTTTATATAATGACTCGGGCTTAAGCGGTTCTCTTCCCCAT
SEQ ID NO: 3: Truncated Bacillus subtilis AmyE (AmyE-tr) amino acid
sequence. The native signal sequence is not shown.
    1 LTAPSIKSGT ILHAWNWSFN TLKHNMKDIH DAGYTAIQTS PINQVKEGNQ
  51 GDKSMSNWYW LYQPTSYQIG NRYLGTEQEF KEMCAAAEEY GIKVIVDAVI
101 NHTTSDYAAI SNEVKSIPNW THGNTQIKNW SDRWDVTQNS LLGLYDWNTQ
151 NTQVQSYLKR FLDRALNDGA DGFRFDAAKH IELPDDGSYG SQFWPNITNT
201 SAEFQYGEIL QDSASRDAAY ANYMDVTASN YGHSIRSALK NRNLGVSNIS
251 HYASDVSADK LVTWVESHDT YANDDEESTW MSDDDIRLGW AVIASRSGST
301 PLFFSRPEGG GNGVRFPGKS QIGDRGSALF EDQAITAVNR FHNVMAGQPE
351 ELSNPNGNNQ IFMNQRGSHG VVLANAGSSS VSINTATKLP DGRYDNKAGA
401 GSFQVNDGKL TGTINARSVA VLYPD
SEQ ID NO: 4: Nucleotide sequence encoding AmyE-tr (SEQ ID NO: 2).
CTTACAGCACCGTCGATCAAAAGCGGAACCATTCTTCATGCATGGAATTGGTCGTTCAATAC
GTTAAAACACAATATGAAGGATATTCATGATGCAGGATATACAGCCATTCAGACATCTCCGA
TTAACCAAGTAAAGGAAGGGAATCAAGGAGATAAAAGCATGTCGAACTGGTACTGGCTGTAT
CAGCCGACATCGTATCAAATTGGCAACCGTTACTTAGGTACTGAACAAGAATTTAAAGAAAT
GTGTGCAGCCGCTGAAGAATATGGCATAAAGGTCATTGTTGACGCGGTCATCAATCATACCA
CCAGTGATTATGCCGCGATTTCCAATGAGGTTAAGAGTATTCCAAACTGGACACATGGAAAC
ACACAAATTAAAAACTGGTCTGATCGATGGGATGTCACGCAGAATTCATTGCTCGGGCTGTA
TGACTGGAATACACAAAATACACAAGTACAGTCCTATCTGAAACGGTTCTTAGACAGGGCAT
TGAATGACGGGGCAGACGGTTTTCGATTTGATGCCGCCAAACATATAGAGCTTCCAGATGAT
GGCAGTTACGGCAGTCAATTTTGGCCGAATATCACAAATACATCAGCAGAGTTCCAATACGG
AGAAATCCTTCAGGATAGTGCCTCCAGAGATGCTGCATATGCGAATTATATGGATGTGACAG
CGTCTAACTATGGGCATTCCATAAGGTCCGCTTTAAAGAATCGTAATCTGGGCGTGTCGAAT
ATCTCCCACTATGCATCTGATGTGTCTGCGGACAAGCTAGTGACATGGGTAGAGTCGCATGA
TACGTATGCCAATGATGATGAAGAGTCGACATGGATGAGCGATGATGATATCCGTTTAGGCT
GGGCGGTGATAGCTTCTCGTTCAGGCAGTACGCCTCTTTTCTTTTCCAGACCTGAGGGAGGC
GGAAATGGTGTGAGGTTCCCGGGGAAAAGCCAAATAGGCGATCGCGGGAGTGCTTTATTTGA
AGATCAGGCTATCACTGCGGTCAATAGATTTCACAATGTGATGGCTGGACAGCCTGAGGAAC
TCTCGAACCCGAATGGAAACAACCAGATATTTATGAATCAGCGCGGCTCACATGGCGTTGTG
CTGGCAAATGCAGGTTCATCCTCTGTCTCTATCAATACGGCAACAAAATTGCCTGATGGCAG
GTATGACAATAAAGCTGGAGCGGGTTCATTTCAAGTGAACGATGGTAAACTGACAGGCACGA
TCAATGCCAGGTCTGTAGCTGTGCTTTATCCTGAT
SEQ ID NO: 5: Bacillus subtilis alpha-amylase variant Amy31A amino acid
sequence (UniProtKB/TrEMBL Accession No. O82953). The native signal
sequence is shown in bold.
    1 MFEKRFKTSL LPLFAGFLLL FHLVLSGPAA ANAETANKSN KVTASSVKNG
  51 TILHAWNWSF NTLTQNMKDI RDAGYAAIQT SPINQVKEGN QGDKSMSNWY
101 WLYQPTSYQI GNRYLGTEQE FKDMCAAAEK YGVKVIVDAV VNHTTSDYGA
151 ISDEIKRIPN WTHGNTQIKN WSDRWDITQN ALLGLYDWNT QNTEVQAYLK
201 GFLERALNDG ADGFRYDAAK HIELPDDGNY GSQFWPNITN TSAEFQYGEI
251 LQDSASRDTA YANYMNVTAS NYGHSIRSAL KNRILSVSNI SHYASDVSAD
301 KLVTWVESHD TYANDDEEST WMSDDDIRLG WAVIGSRSGS TPLFFSRPEG
351 GGNGVRFPGK SQIGDRGSAL FKDQAITAVN QFHNEMAGQP EELSNPNGNN
401 QIFMNQRGSK GVVLANAGSS SVTINTSTKL PDGRYDNRAG AGSFQVANGK
451 LTGTINARSA AVLYPDDIGN APHVFLENYQ TEAVHSFNDQ LTVTLRANAK
501 TTKAVYQINN GQETAFKDGD RLTIGKEDPI GTTYNVKLTG TNGEGASRTQ
551 EYTFVKKDPS QTNIIGYQNP DHWGNVNAYI YKHDGGGAIE LTGSWPGKAM
601 TKNADGIYTL TLPANADTAD AKVIFNNGSA QVPGQNHPGF DYVQNGLYNN
651 SGLNGYLPH
SEQ ID NO: 6: Nucleotide sequence encoding B. subtilis Amy31A
(SEQ ID NO: 3).
TCTGTTAAAAACGGCACTATTCTGCATGCATGGAACTGGAGCTTTAACACGCTGACCCAGAA
CATGAAAGATATTCGTGACGCGGGCTATGCTGCGATCCAAACCAGCCCTATCAACCAGGTCA
AAGAAGGCAACCAAGGCGACAAATCCATGTCCAACTGGTACTGGCTGTATCAACCGACGTCC
TATCAGATTGGCAACCGTTATCTGGGCACGGAGCAAGAGTTCAAAGACATGTGTGCTGCGGC
TGAGAAATATGGTGTGAAAGTTATCGTGGACGCTGTGGTAAACCACACGACCTCTGATTATG
GTGCTATTAGCGACGAGATTAAACGTATTCCAAATTGGACCCATGGTAATACCCAGATCAAA
AATTGGAGCGACCGCTGGGACATTACCCAGAATGCGCTGCTGGGTCTGTATGACTGGAACAC
GCAAAACACCGAAGTACAGGCATATCTGAAGGGCTTCCTGGAACGCGCTCTGAACGATGGTG
CTGATGGTTTTCGCTACGACGCCGCAAAGCATATTGAGCTGCCGGATGACGGCAACTACGGT
TCCCAATTCTGGCCGAACATCACCAACACCTCTGCCGAATTCCAGTACGGCGAGATCCTGCA
AGACTCCGCGAGCCGTGACACCGCTTATGCCAACTATATGAACGTAACTGCCTCTAACTATG
GCCATTCCATTCGTTCTGCGCTGAAAAATCGTATCCTGTCCGTGTCCAATATCTCCCACTAT
GCATCCGACGTTTCTGCTGACAAACTGGTAACTTGGGTCGAGTCTCACGACACCTATGCAAA
TGATGACGAGGAGAGCACCTGGATGAGCGATGATGATATTCGTCTGGGTTGGGCGGTTATTG
GTTCTCGCTCTGGTTCTACTCCGCTGTTCTTTAGCCGTCCGGAAGGTGGCGGCAATGGCGTT
CGTTTCCCGGGTAAATCTCAAATTGGTGATCGTGGCTCTGCACTGTTTAAAGATCAAGCTAT
TACGGCGGTGAATCAGTTCCATAATGAGATGGCAGGTCAACCTGAAGAACTGTCCAATCCAA
ACGGTAACAACCAAATCTTCATGAACCAGCGTGGCAGCAAAGGCGTCGTCCTGGCGAACGCC
GGTAGCTCTTCTGTTACCATCAACACGTCTACCAAACTGCCAGACGGCCGCTATGATAACCG
TGCGGGTGCTGGTTCCTTTCAGGTAGCCAACGGCAAGCTGACGGGCACCATCAACGCTCGTT
CTGCTGCTGTTCTGTACCCGGACGACATTGGCAACGCTCCGCACGTGTTCCTGGAGAATTAC
CAGACCGAAGCGGTACATAGCTTTAATGACCAGCTGACCGTCACTCTGCGTGCCAACGCAAA
AACCACGAAAGCAGTCTATCAGATCAATAATGGTCAAGAAACTGCTTTCAAGGATGGCGACC
GTCTGACTATTGGTAAGGAGGACCCGATTGGCACCACTTATAACGTTAAACTGACTGGCACC
AATGGCGAGGGCGCTAGCCGCACTCAAGAGTATACGTTCGTAAAGAAAGACCCGTCTCAAAC
CAACATCATCGGTTACCAGAATCCTGACCACTGGGGTAATGTGAACGCTTACATCTATAAAC
ATGATGGTGGCGGTGCTATCGAACTGACCGGCTCTTGGCCAGGTAAAGCCATGACGAAAAAC
GCGGATGGCATCTATACCCTGACCCTGCCGGCCAATGCGGATACCGCAGATGCGAAGGTTAT
CTTCAATAACGGCTCCGCGCAGGTTCCGGGCCAAAACCATCCGGGCTTTGACTACGTACAAA
ATGGTCTGTATAACAACTCTGGCCTGAACGGTTACCTGCCGCAC
SEQ ID NO: 7: Full length Geobacillus stearothermophilus alpha-amylase
(AmyS; P06279) protein sequence. The signal sequence is shown in bold.
    1 MLTFHRIIRK GWMFLLAFLL TALLFCPTGQ PAKAAAPFNG TMMQYFEWYL
  51 PDDGTLWTKV ANEANNLSSL GITALWLPPA YKGTSRSDVG YGVYDLYDLG
101 EFNQKGAVRT KYGTKAQYLQ AIQAAHAAGM QVYADVVFDH KGGADGTEWV
151 DAVEVNPSDR NQEISGTYQI QAWTKFDFPG RGNTYSSFKW RWYHFDGVDW
201 DESRKLSRIY KFRGIGKAWD WEVDTENGNY DYLMYADLDM DHPEVVTELK
251 SWGKWYVNTT NIDGFRLDAV KHIKFSFFPD WLSDVRSQTG KPLFTVGEYW
301 SYDINKLHNY IMKTNGTMSL FDAPLHNKFY TASKSGGTFD MRTLMTNTLM
351 KDQPTLAVTF VDNHDTEPGQ ALQSWVDPWF KPLAYAFILT RQEGYPCVFY
401 GDYYGIPQYN IPSLKSKIDP LLIARRDYAY GTQHDYLDHS DIIGWTREGV
451 TEKPGSGLAA LITDGPGGSK WMYVGKQHAG KVFYDLTGNR SDTVTINSDG
501 WGEFKVNGGS VSVWVPRKTT VSTIAWSITT RPWTDEFVRW TEPRLVAWP
SEQ ID NO: 8: Full length Bacillus licheniformis alpha-amylase
(AmyL; P06278) protein sequence. The signal sequence is shown in bold.
    1 MKQQKRLYAR LLTLLFALIF LLPHSAAAAA NLNGTLMQYF EWYMPNDGQH
  51 WKRLQNDSAY LAEHGITAVW IPPAYKGTSQ ADVGYGAYDL YDLGEFHQKG
101 TVRTKYGTKG ELQSAIKSLH SRDINVYGDV VINHKGGADA TEDVTAVEVD
151 PADRNRVISG EHRIKAWTHF HFPGRGSTYS DFKWHWYHFD GTDWDESRKL
201 NRIYKFQGKA WDWEVSNENG NYDYLMYADI DYDHPDVAAE IKRWGTWYAN
251 ELQLDGFRLD AVKHIKFSFL RDWVNHVREK TGKEMFTVAE YWQNDLGALE
301 NYLNKTNFNH SVFDVPLHYQ FHAASTQGGG YDMRKLLNST VVSKHPLKAV
351 TFVDNHDTQP GQSLESTVQT WFKPLAYAFI LTRESGYPQV FYGDMYGTKG
401 DSQREIPALK HKIEPILKAR KQYAYGAQHD YFDHHDIVGW TREGDSSVAN
451 SGLAALITDG PGGAKRMYVG RQNAGETWHD ITGNRSEPVV INSEGWGEFH
501 VNGGSVSIYV QR
SEQ ID NO: 9: Full length Bacillus subtilis alpha-amylase (AmyE; NP_388186).
The signal sequence is shown in bold.
    1 MFAKRFKTSL LPLFAGFLLL FHLVLAGPAA ASAETANKSN ELTAPSIKSG
  51 TILHAWNWSF NTLKHNMKDI HDAGYTAIQT SPINQVKEGN QGDKSMSNWY
101 WLYQPTSYQI GNRYLGTEQE FKEMCAAAEE YGIKVIVDAV INHTTSDYAA
151 ISNEVKSIPN WTHGNTQIKN WSDRWDVTQN SLLGLYDWNT QNTQVQSYLK
201 RFLDRALNDG ADGFRFDAAK HIELPDDGSY GSQFWPNITN TSAEFQYGEI
251 LQDSASRDAA YANYMDVTAS NYGHSIRSAL KNRNLGVSNI SHYASDVSAD
301 KLVTWVESHD TYANDDEEST WMSDDDIRLG WAVIASRSGS TPLFFSRPEG
351 GGNGVRFPGK SQIGDRGSAL FEDQAITAVN RFHNVMAGQP EELSNPNGNN
401 QIFMNQRGSH GVVLANAGSS SVSINTATKL PDGRYDNKAG AGSFQVNDGK
451 LTGTINARSV AVLYPDDIAK APHVFLENYK TGVTHSFNDQ LTITLRADAN
501 TTKAVYQINN GPDDRRLRME INSQSEKEIQ FGKTYTIMLK GTNSDGVTRT
551 EKYSFVKRDP ASAKTIGYQN PNHWSQVNAY IYKHDGSRVI ELTGSWPGKP
601 MTKNADGIYT LTLPADTDTT NAKVIFNNGS AQVPGQNQPG FDYVLNGLYN
651 DSGLSGSLPH
SEQ ID NO: 10: Full length Bacillus subtilis alpha-amylase (AmyE; NCBI
Accession No. ABW75769)
    1 MFAKRFKTSL LPLFAGFLLL FHLVLAGPAA ASAETANKSN ELTAPSIKSG
  51 TILHAWNWSF NTLKHNMKDI HDAGYTAIQT SPINQVKEGN QGNKSMSNWY
101 WLYQPTSYQI GNRYLGTEQE FKEMCAAAEE YGIKVIVDAV INHTTSDYAA
151 ISNEIKSIPN WTHGNTQIKN WSDRWDVTQN SLLGLYDWNT QNTQVQSYLK
201 RFLERALNDG ADGFRFDAAK HIELPDDGSY GSQFWPNITN TSAEFQYGEI
251 LQDSASRDAA YANYMNVTAS NYGHSIRSAL KNRNLGVSNI SHYASDVSAD
301 KLVTWVESHD TYANDDEEST WMSDDDIRLG WAVIASRSGS TPLFFSRPEG
351 GGNGVRFPGK SQIGDRGSAL FEDQAITAVN RFHNVMAGQP EELSNPNGNN
401 QIFMNQRGSH GVVLANAGSS SVSINTPTKL PDGRYDNKAG AGSFQVNDGK
451 LTGTINARSV AVLYPDDIAK APHVFLENYK TGVTHSFNDQ LTITLRADAN
501 TTKAVYQINN GPETAFKDGD QFTIGKGDPF GKTYTIMLKG TNSNGVTKAE
551 EYSFVKRDPA SAKTIGYQNP NHWSQVNAYI YKHDGSRAIE LTGSWPGKPM
601 TKNADGIYTL TLPADTDTTN AKVIFNNGSA QVPGQNQPGF DYVQNGLYND
651 SGLSGSLPH
SEQ ID NO: 11: Full length Bacillus subtilis alpha-amylase (AmyE; NCBI
Accession No. ABK54355)
    1 MFAKRFKTSL LPLFAGFLLL FHLVLAGPAA ASAETANKSN ELTAPSIKSG
  51 TILHAWNWSF NTLKHNMKDI HDAGYTAIQT SPINQVKEGN QGDKSMSNWY
101 WLYQPTSYQI GNRYLGTEQE FKEMCAAAEE YGIKVIVDAV INHTTSDYAA
151 ISNEIKSIPN WTHGNTQIKN WSDRWDVTQN SLLGLYDWNT QNTQVQSYLK
201 RFLERALNDG ADGFRFDAAK HIELPDDGSY GSQFWPTITN TSAEFQYGEI
251 LQDSASRDAA YANYMDVTAS NYGHSIRSAL KNRNLGVSNL SHYASDVSAD
301 KLVTWVESHD TYANDDEEST WMSDDDIRLG WAVIASRSGS TPLFFSRPEG
351 GGNGVRFPGK SQIGDRGSAL FEDQAITAVN RFHNVMAGQP EELSNPNGNN
401 QIFMNQRGSH GVVLANAGSS SVSINTATKL PDGRYDNKAG AGSFQVNDGK
451 LTGTINARSV AVLYPDDIAK APHVFLENYK TGVTHSFNDQ LTITLRADAN
501 TTKAVYQINN GPETAFKDGD QFTIGKGDPF GKTYTIMLKG TNSDGVTRAE
551 EYSFVKRDPA SAKTIGYQNP NHWSQVNAYI YKHDGGRAIE LTGSWPGKPM
601 TKNADGIYTL TLPADTDTTN AKVIFNNGSA QVPGQNQPGF DYVQNGLYND
651 SGLSGSLPH
SEQ ID NO: 12: Full length Bacillus subtilis alpha-amylase (AmyE; NCBI
Accession No. AAF14358)
    1 MFAKRFKTSL LPLFAGFLLL FHLVLAGPAA ASAETANKSN ELTAPSIKSG
  51 TILHAWNWSF NTLKHNMKDI HDAGYTAIQT SPINQVKEGN QGDKSMSNWY
101 WLYQPTSYQI GNRYLGTEQE FKEMCAAAEE YGIKVIVDAV INHTTSDYAA
151 ISNEIKSIPN WTHGNTQIKN WSDRWDVTQN SLLGLYDWNT QNTQVQSYLK
201 RFLERALNDG ADGFRFDAAK HIELPDDGSY GSQFWPTITN TSAEFQYGEI
251 LQDSASRDAA YANYMDVTAS NYGHSIRSAL KNRNLGVSNL SHYASDVSAD
301 KLVTWVESHD TYANDDEEST WMSDDDIRLG WAVIASRSGS TPLFFSRPEG
351 GGNGVRFPGK SQIGDRGSAL FEDQAITAVN RFHNVMAGQP EELSNPNGNN
401 QIFMNQRGSH GVVLANAGSS SVSINTATKL PDGRYDNKAG AGSFQVNDGK
451 LTGTINARSV AVLYPDDIAK APHVFLENYK TGVTHSFNDQ LTITLRADAN
501 TTKAVYQINN GPETAFKDGD QFTIGKGDPF GKTYTIMLKG TNSDGVTRAE
551 EYSFVKRDPA SAKTIGYQNP NHWSQVNAYI YKHDGGRAIE LTGSWPGKPM
601 TKNADGIYTL TLPADTDTTN AKVIFNNGSA QVPGQNQPGF DYVQNGLYND
651 SGLSGSLPH
SEQ ID NO: 13: Full length Bacillus subtilis alpha-amylase (AmyE; NCBI
Accession No. AAT01440)
    1 MFAKRFKTSL LPLFAGFLLL FHLVLAGPAA ASAETANKSN ELTAPSIKSG
  51 TILHAWNWSF NTLKHNMKDI HDAGYTAIQT SPINQVKEGN QGDKSMSNWY
101 WLYQPTSYQI GNRYLGTEQE FKEMCAAAEE YGIKVIVDAV INHTTSDYAA
151 ISNEVKSIPN WTHGNTQIKN WSDRWDVTQN SLLGLYDWNT QNTQVQSYLK
201 RFLERALNDG ADGFRFDAAK HIELPDDGSY GSQFWPNITN TSAEFQYGEI
251 LQDSASRDAA YANYMDVTAS NYGHSIRSAL KNRNLGVSNI SHYASDVSAD
301 KLVTWVESHD TYANDDEEST WMSDDDIRLG WAVIASRSGS TPLFFSRPEG
351 GGNGVRFPGK SQIGDRGSAL FEDQAITAVN RFHNVMAGQP EELSNPNGNN
401 QIFMNQRGSH GVVLANAGSS SVSINTPTKL PDGRYDNKAG AGSFQVNDGK
451 LTGTINARSV AVLYPDDIAQ APHVFLENYK TGVTHSFNDQ LTITLRADAN
501 TTKAVYQINN GPETAFKDGD QFTIGKGDPF GKTYTIMLKG TNSDGVTRTE
551 EYSFIKRDPA SAKTIGYQNP NHWSQVNAYI YKHDGGQAIE LTGSWPGKPM
601 TKNADGIYTL TLPADTDTTN AKVIFNNGSA QVPGQNQPGF DYVQNGLYND
651 SGLSGSLPY
SEQ ID NO: 14: Full length Bacillus subtilis alpha-amylase (AmyE; NCBI
Accession No. AAZ30064)
    1 MFAKRFKTSL LPLFAGFLLL FHLVLAGPNA ANAETANKSN ELTAPSIKSG
  51 TILHAWNWSF NTLKHNMKDI HDAGYTAIQT SPINQVKEGN QGNKSMLNWY
101 WLYQPTSYQI GNRYLGTEQE FKEMCAAAEE YGIKVIVDAV INHTTSDYAA
151 ISNEIKSIPN WTHGNTQIKN WSDRWDVTQN SLLGLYDWNT QNTQVQSYLK
201 RFLERALNDG ADGFRFDAAK HIELPDDGSY GSQFWPNITN TSAEFQYGEI
251 LQDSASRDAS YANYMNVTAS NYGHSIRSAL KNRNLGVSNI SHYASDVPAD
301 KLVTWVESHD TYANDDEEST WMSDDDIRLG WAVIASRSGS TPLFFSRPEG
351 GGNGVRFPGK SQIGDRGSAL FEDQAITAVN RFHNVMAGQP EELSNPNGNN
401 QIFMNQRGSH GVVLANAGSS SVSINTPTKL PDGRYDNKAG AGSFQVNDGK
451 LTGTINARSV AVLYPDDIAK APHVFLENYK TGVTHSFNDQ LTITMRADAK
501 TTKAVYQINN GPETAFKDGD QFTIGKGDPF GKTYTIMLKG TNSDGVTRTE
551 EYSFIKRDPA SAKTIGYQNP NHWSQVNAYI YKHDGGQAIE LTGSWPGKPM
601 TKNADGIYTL TLPADTDTTN AKVIFNNGSA QVPGQNQPGF DYVQNGLYND
651 SGLSGSLPH
SEQ ID NO: 15: Full length Bacillus subtilis alpha-amylase (AmyE: NCBI
Accession No. AAQ83841)
    1 MFAKRFKTSL LPLFAGFLLL FYLVLAGPAA ASAETANKSI ELTAPSIKSG
  51 TILHAWNWSF NTLKHNMKDI HDAGYTAIQT SPINQVKEGN QGDKSMSNWY
101 WLYQPTSYQI GNRYLGTEQE FKEMCAAAEE YGIKVIVDAV INHTTSDYAA
151 ISNEVKSIPN WTHGNTQIKN WSDRWDVTQN SLLGLYDWNT QNTQVQSYLK
201 RFLDRALNDG ADGFRFDAAK HIELPDDGSY GSQFWPNITN TSAEFQYGEI
251 LQDSASRDAA YANYMDVTAS NYGHSIRSAL KNRNLGVSNI SHYASDVSAD
301 KLVTWVESHD TYANDDEEST WMSDDDIRLG WAVIASRSGS TPLFFSRPEG
351 GGNGVRFPGK SQIGDRGSAL FEDQAITAVN RFHNVMAGQP EELSNPNGNN
401 QIFMNQRISH GVVLANAGSS SVSINTATKL PDGRYDNKAG AGSFQVNDGK
451 LTGTINARSV AVLYPDDIAK APHVFLENYK TGVTHSFNDQ LTITLRADAN
501 TFIKSIMDQI NXRXRRLRME INSQSEKEIQ FGKTYTIMLK GTNSDGVTRX
551 EKYSLPKRDP ASAKTIGYQN PNHWSQVNAY IYKHDGSREI ELTGSWPGKP
601 MTKNADGIYT LTLPADTDTT NAKVIFNNGY AQVPGQNQPG FDYVLNGLY
SEQ ID NO: 16: Full length Bacillus subtilis alpha-amylase (AmyE; NCBI
Accession No. BAA31528)
    1 MFEKRFKTSL LPLFAGFLLL FHLVLSGPAA ANAETANKSN KVTASSVKNG
  51 TILHAWNWSF NTLTQNMKDI RDAGYAAIQT SPINQVKEGN QGDKSMSNWY
101 WLYQPTSYQI GNRYLGTEQE FKDMCAAAEK YGVKVIVDAV VNHTTSDYGA
151 ISDEIKRIPN WTHGNTQIKN WSDRWDITQN ALLGLYDWNT QNTEVQAYLK
201 GFLERALNDG ADGFRYDAAK HIELPDDGNY GSQFWPNITN TSAEFQYGEI
251 LQDSASRDTA YANYMNVTAS NYGHSIRSAL KNRILSVSNI SHYASDVSAD
301 KLVTWVESHD TYANDDEEST WMSDDDIRLG WAVIGSRSGS TPLFFSRPEG
351 GGNGVRFPGK SQIGDRGSAL FKDQAITAVN QFHNEMAGQP EELSNPNGNN
401 QIFMNQRGSK GVVLANAGSS SVTINTSTKL PDGRYDNRAG AGSFQVANGK
451 LTGTINARSA AVLYPDDIGN APHVFLENYQ TEAVHSFNDQ LTVTLRANAK
501 TTKAVYQINN GQETAFKDGD RLTIGKEDPI GTTYNVKLTG TNGEGASRTQ
551 EYTFVKKDPS QTNIIGYQNP DHWGNVNAYI YKHDGGGAIE LTGSWPGKAM
601 TKNADGIYTL TLPANADTAD AKVIFNNGSA QVPGQNHPGF DYVQNGLYNN
651 SGLNGYLPH
SEQ ID NO: 17: Native signal sequence of the AmyE of SEQ ID NO: 1.
MFAKRFKTSLLPLFAGFLLLFHLVLAGPAAASAETANKSNE
SEQ ID NO: 18: Primer PSTAMYE-F
CTTCTTGCTGCCTCATTCTGCAGCTTCAGCACTTACAGCACCGTCGATCAAAAGCGGAAC
SEQ ID NO: 19: Primer AMYENOPST-R
CTGGAGGCACTATCCTGAAGGATTTCTCCGTATTGGAACTCTGCTGATGTATTTGTG
SEQ ID NO: 20: Primer AMYENOPST-F
CACAAATACATCAGCAGAGTTCCAATACGGAGAAATCCTTCAGGATAGTGCCTCCAG
SEQ ID NO: 21: Primer HPAIAMYE-R
CAGGAAATCCGTCCTCTGTTAACTCAATGGGGAAGAGAACCGCTTAAGCCCGAGTC
SEQ ID NO: 22: Primer HPAIAMYE466-R
CAGGAAATCCGTCCTCTGTTAACTCAATCAGGATAAAGCACAGCTACAGACCTGG
SEQ ID NO: 23: Primer AMYE SEQ-F1
TACACAAGTACAGTCCTATCTG
SEQ ID NO: 24: Primer AMYE SEQ-F2
CATCCTCTGTCTCTATCAATAC
```

What is claimed is:

1. A method of eliminating or reducing iodine-positive starch in a saccharide liquor, comprising contacting an alpha-amylase with a saccharide liquor produced from a saccharification process and containing iodine-positive starch, wherein the alpha-amylase is a Bacillus subtilis alpha-amylase (AmyE) having an amino acid sequence of SEQ ID NO: 1 or an alpha-amylase having at least about 90% sequence identity to SEQ ID NO: 1, and wherein the alpha-amylase is effective eliminating or reducing iodine-positive starch present in the saccharide liquor.

2. The method of claim 1, wherein the alpha-amylase comprises an amino acid sequence set forth in SEQ ID NO: 3, SEQ ID NO: 5, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16.

3. The method of claim 1, wherein the alpha-amylase comprises SEQ ID NO: 1.

4. The method of claim 1, wherein the alpha-amylase consists of SEQ ID NO: 1.

5. The method of claim 1, wherein the alpha-amylase is an AmyE variant.

6. The method of claim 2, wherein the AmyE variant has one or more altered properties compared to the AmyE having an amino acid sequence of SEQ ID NO: 1.

7. The method of claim 6, wherein the one or more altered properties of the alpha-amylase is: substrate specificity, substrate binding, substrate cleavage pattern, thermal stability, pH/activity profile, pH/stability profile, stability towards oxidation, stability at lower level of calcium ion (Ca²⁺), specific activity, or any combination thereof.

8. The method of claim 1, wherein the alpha-amylase is used at an amount of about 0.1 to about 0.4 mg per gram of starch (mg/g starch).

9. The method of claim 1, wherein the elimination or reduction of iodine-positive starch is performed at a pH about 5.0 to about 5.5.

10. The method of claim 1, wherein the elimination or reduction of iodine-positive starch is performed at a temperature about 58° C. to about 62° C.

11. The method of claim 1, wherein the elimination or reduction of iodine-positive starch is performed for about 4 to about 24 hours.

12. The method of claim 1, wherein the iodine-positive starch results from process excursions of temperature, pH, enzyme dose, or any combination thereof.

13. The method of claim 1 further comprising contacting a phytase with the saccharide liquor.

14. A composition for eliminating or reducing iodine-positive starch in a saccharide liquor produced from a saccharification process, comprising an alpha-amylase, wherein the alpha-amylase is a Bacillus subtilis alpha-amylase (AmyE) having an amino acid sequence of SEQ ID NO: 1 or the alpha-amylase having at least about 90% sequence identity to SEQ ID NO: 1, and wherein the alpha-amylase reduces iodine-positive starch present in the saccharide liquor.

15. The composition of claim 14, wherein the alpha-amylase comprises an amino acid sequence set forth in SEQ ID NO: 3, SEQ ID NO: 5, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16.

16. The composition of claim 14, wherein the alpha-amylase comprises SEQ ID NO: 1.

17. The composition of claim 14, wherein the alpha-amylase consists of SEQ ID NO: 1.

18. The composition of claim 14, wherein the alpha-amylase is a variant of AmyE.

19. The composition of claim 15, wherein the AmyE variant has one or more altered properties compared to the AmyE having an amino acid sequence of SEQ ID NO: 1.

20. The composition of claim 19, wherein the one or more altered properties of the alpha-amylase is: substrate specificity, substrate binding, substrate cleavage pattern, thermal stability, pH/activity profile, pH/stability profile, stability towards oxidation, stability at lower level of calcium ion (Ca²⁺), specific activity, or any combination thereof.

21. A method of eliminating or reducing iodine-positive starch in a saccharide liquor comprising contacting the composition of claim 14 with a saccharide liquor produced from a saccharification process and containing iodine-positive starch to reduce iodine-positive starch present in the saccharide liquor.

22. The method of claim 21, wherein the alpha-amylase is used at an amount of about 0.1 to about 0.4 mg per gram of starch (mg/g starch).

23. The method of claim 21, wherein the elimination or reduction of iodine-positive starch is performed at a pH about 5.0 to about 5.5.

24. The method of claim 21, wherein the elimination or reduction of iodine-positive starch is performed at a temperature about 58° C. to about 62° C.

25. The method of claim 21, wherein the elimination or reduction of iodine-positive starch is performed for about 4 to about 24 hours.

26. The method of claim 21, wherein the iodine-positive starch results from process excursions of temperature, pH, enzyme dose, or any combination thereof.
